SEQUENCE LISTING (274 sequences)

SEQ ID NO: 1 - protein, 212 aa, Homo sapiens
MHPGSPSAWPPRARAALRLWLGCVCFALVQADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 2 - protein, 112 aa, Homo sapiens
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 3 - protein, 141 aa, Ruminococcus torques
APVDVKVSEITETSAKASWKNAADGKEAAGYNVYVDGVKLNKELLTEASCGLTNLKAETTYFVEVTAVDAAGNESVKSEKVTFKTLKAEEQKEDSTLENNEKPGAVQTGDHVNVFVWMIGLLISASAAVAVMFKRNKNRKD

SEQ ID NO: 4 - protein, 87 aa, Ruminococcus torques
APVDVKVSEITETSAKASWKNAADGKEAAGYNVYVDGVKLNKELLTEASCGLTNLKAETTYFVEVTAVDAAGNESVKSEKVTFKTLK

SEQ ID NO: 5 - protein, 21 aa, Ruminococcus torques
AETTYFVEVTAVDAAGNESVK

SEQ ID NO: 6 - protein, 10 aa, Ruminococcus torques
VSEITETSAK

SEQ ID NO: 7 - protein, 13 aa, Ruminococcus torques
EAAGYNVYVDGVK

SEQ ID NO: 8 - protein, 14 aa, Ruminococcus torques
ELLTEASCGLTNLK

SEQ ID NO: 9 - DNA, 261 bp, Ruminococcus torques
gctccggtcgacgtaaaagtttcggaaattaccgagacaagcgcaaaagcatcatggaagaacgcggcggacggaaaagaagcggcaggatacaacgtatatgttgacggtgtaaaattgaacaaagaacttcttacagaagcatcttgcggattgacaaatctgaaagcagagactacatattttgtagaagtgacggcggtagacgcagccggaaatgaatctgttaaatcagaaaaagtgacgtttaagacattgaaa

SEQ ID NO: 10 - DNA, 261 bp, Artificial
  Ruminococcus torques RUCILP2 peptide sequence, codon-optimized for Escherichia coli
  misc_feature (1)..(261): Codon-optimized RUCILP2
gctcccgtagatgttaaagtatcagaaattaccgagaccagcgcgaaagcgagctggaaaaatgcggcggacggtaaagaggcggcgggctacaacgtgtatgttgatggtgtgaagctgaacaaagagctgctgaccgaagcgagctgcggcctgaccaacctgaaagcggaaaccacctacttcgtggaagttaccgcggtggatgcggcgggcaatgagagcgttaagagcgagaaagtgaccttcaagaccctgaaa

SEQ ID NO: 11 - DNA, 63 bp, Ruminococcus torques
gcagagactacatattttgtagaagtgacggcggtagacgcagccggaaatgaatctgttaaa

SEQ ID NO: 12 - DNA, 63 bp, Artificial
  Polypeptide 21-AABP derived from RUCILP2 from Ruminococcus torques, codon-optimized for Escherichia coli
  misc_feature (1)..(63): Codon-optimized 21-AABP derived from RUCILP2
gctgagacaacgtattttgttgaagtaactgctgtggacgcggcaggtaacgagagcgttaag

SEQ ID NO: 13 - DNA, 30 bp, Ruminococcus torques
gtttcggaaattaccgagacaagcgcaaaa

SEQ ID NO: 14 - DNA, 30 bp, Artificial
  Trypsin digested polypeptide derived from amino acid 7-16 of RUCILP2 from Ruminococcus torques, codon-optimized for Escherichia coli
  misc_feature (1)..(30): Derived from amino acid 7-16 of RUCILP2
gtttctgagatcaccgagacgagcgcaaaa

SEQ ID NO: 15 - DNA, 39 bp, Ruminococcus torques
gaagcggcaggatacaacgtatatgttgacggtgtaaaa

SEQ ID NO: 16 - DNA, 39 bp, Artificial
  Trypsin digested polypeptide derived from amino acid 27-39 of RUCILP2 from Ruminococcus torques, codon-optimized for Escherichia coli
  misc_feature (1)..(39): Derived from amino acid 27-39 of RUCILP2
gaagctgccgggtacaacgtctatgtagatggtgtcaaa

SEQ ID NO: 17 - DNA, 42 bp, Ruminococcus torques
gaacttcttacagaagcatcttgcggattgacaaatctgaaa

SEQ ID NO: 18 - DNA, 42 bp, Artificial
  Trypsin digested polypeptide derived from amino acid 43-56 of RUCILP2 from Ruminococcus torques, codon-optimized for Escherichia coli
  misc_feature (1)..(42): Derived from amino acid 43-56 of RUCILP2
gaactgctgacagaagcaagttgcggacttactaaccttaaa

SEQ ID NO: 19 - protein, 88 aa, Ruminococcus torques
APVDVKVSEVTETSAKVSWKNAADGKEAAGYNVYVDGTKVNEELIAETTYNVSSLKDGTTYSVEVTAVDAAGEESAKSEKVEFTTVKK

SEQ ID NO: 20 - protein, 21 aa, Ruminococcus torques
DGTTYSVEVTAVDAAGEESAK

SEQ ID NO: 21 - protein, 1271 aa, Ruminococcus torques
MKKKWVSGMLALLLVGTTVGSMMPTEAVQAEENSQGKTYYVDSENGKDTNDGLSEGKAFQTLNKVNDLTLGAGDRVLLKNGSVFEDQALHIKGSGSENAPIKISTYGDEKDGRPQINTNGHGQWELNYGHKLDNQNHKWHGTVSSSILLKDVEYIEIEGLEITNDRDSATDAEKDKNYKYNDAECMDRTGVAGVAKNKGTVDHIVLNDLYIHDVTGNVYNKHMTNGGIYFIVEKPENESATGIARYNDVTIQNCYLDTVNRWGIAVGYTYEWAKFNGGALSDETMKTYASSDVVIQNNYLNNVGGDAITTMYIDRPVIQYNVSENAAAQINTTDYTDPQPQLDANGNPNGKTHTGGRVAAGIWPWKCKNAVFQYNECFKTLNASKGNGDGQPWDADYGDGTNYQYNYSHGNTASTIMFCGGESINNTFRYNISVHEDMGPLDPAGNAGNTQVYNNTFVIKEGIRSIWYRDSGPVTMENNIFYFDGEQPAQITNWNPRNNKVYSNNLFYNVSSYPDDKAAVKVEKGTPVLADAASGPVKAAENKQARRHEDPTEITVFDGFKLAENSPAINKGKVVIDRNGYSIDHDFFGHAVTATPEIGAAESDVIGDLVLRSVVYQIDQESKTISDIPKNTTVEQFCKDSIVDTGVTITVKSKDGKPLENADIIKGGMTVTVSCEGKEAVVYTVVASSDNKLKSAYYEVKDKTIYVPFTEKNPTTAGELKGNVQAAETAEVSVVSGEKTLKDQENIADAMTMRITAEDGKTNDYTIKQKNTYNWALDYAGPQQGNVWFGQKKAASGEWTEIKEYDSQYPNWMVNTYYGPGIDEQSHSAKPTEATHGLLSAPPSTGISTAMAYRVPKDGMVSFHVKDDEPYLRQNGNSGGTVTLKLLVNDEEKQSVILEQSKVQAKDWKAFDKIEVKRGDYIRVAAISNGNPTKPSVHVTPIITYLNEGGTPAPEPTPELKAPVDVKVSEVTETSAKVSWKNAADGKEAAGYNVYVDGTKVNEELIAETTYNVSSLKDGTTYSVEVTAVDAAGEESAKSEKVEFTTVKKVVVDKEALKANIERASALLNETDKYTEESLKALEEALAAAQKVNSDPKADQTKVNDANTALEKAIKDLKEQEKPDPEPTPELKAPVDVKVSEITETSAKASWKNAADGKEAAGYNVYVDGVKLNKELLTEASCGLTNLKAETTYFVEVTAVDAAGNESVKSEKVTFKTLKAEEQKEDSTLENNEKPGAVQTGDHVNVFVWMIGLLISASAAVAVMFKRNKNR

SEQ ID NO: 22 - protein, 15 aa, Ruminococcus torques
APVDVKVSEVTETSA

SEQ ID NO: 23 - protein, 15 aa, Ruminococcus torques
PVDVKVSEVTETSAK

SEQ ID NO: 24 - protein, 15 aa, Ruminococcus torques
VDVKVSEVTETSAKV

SEQ ID NO: 25 - protein, 15 aa, Ruminococcus torques
DVKVSEVTETSAKVS

SEQ ID NO: 26 - protein, 15 aa, Ruminococcus torques
VKVSEVTETSAKVSW

SEQ ID NO: 27 - protein, 15 aa, Ruminococcus torques
KVSEVTETSAKVSWK

SEQ ID NO: 28 - protein, 15 aa, Ruminococcus torques
VSEVTETSAKVSWKN

SEQ ID NO: 29 - protein, 15 aa, Ruminococcus torques
SEVTETSAKVSWKNA

SEQ ID NO: 30 - protein, 15 aa, Ruminococcus torques
EVTETSAKVSWKNAA

SEQ ID NO: 31 - protein, 15 aa, Ruminococcus torques
VTETSAKVSWKNAAD

SEQ ID NO: 32 - protein, 15 aa, Ruminococcus torques
TETSAKVSWKNAADG

SEQ ID NO: 33 - protein, 15 aa, Ruminococcus torques
ETSAKVSWKNAADGK

SEQ ID NO: 34 - protein, 15 aa, Ruminococcus torques
TSAKVSWKNAADGKE

SEQ ID NO: 35 - protein, 15 aa, Ruminococcus torques
SAKVSWKNAADGKEA

SEQ ID NO: 36 - protein, 15 aa, Ruminococcus torques
AKVSWKNAADGKEAA

SEQ ID NO: 37 - protein, 15 aa, Ruminococcus torques
KVSWKNAADGKEAAG

SEQ ID NO: 38 - protein, 15 aa, Ruminococcus torques
VSWKNAADGKEAAGY

SEQ ID NO: 39 - protein, 15 aa, Ruminococcus torques
SWKNAADGKEAAGYN

SEQ ID NO: 40 - protein, 15 aa, Ruminococcus torques
WKNAADGKEAAGYNV

SEQ ID NO: 41 - protein, 15 aa, Ruminococcus torques
KNAADGKEAAGYNVY

SEQ ID NO: 42 - protein, 15 aa, Ruminococcus torques
NAADGKEAAGYNVYV

SEQ ID NO: 43 - protein, 15 aa, Ruminococcus torques
AADGKEAAGYNVYVD

SEQ ID NO: 44 - protein, 15 aa, Ruminococcus torques
ADGKEAAGYNVYVDG

SEQ ID NO: 45 - protein, 15 aa, Ruminococcus torques
DGKEAAGYNVYVDGT

SEQ ID NO: 46 - protein, 15 aa, Ruminococcus torques
GKEAAGYNVYVDGTK

SEQ ID NO: 47 - protein, 15 aa, Ruminococcus torques
KEAAGYNVYVDGTKV

SEQ ID NO: 48 - protein, 15 aa, Ruminococcus torques
EAAGYNVYVDGTKVN

SEQ ID NO: 49 - protein, 15 aa, Ruminococcus torques
AAGYNVYVDGTKVNE

SEQ ID NO: 50 - protein, 15 aa, Ruminococcus torques
AGYNVYVDGTKVNEE

SEQ ID NO: 51 - protein, 15 aa, Ruminococcus torques
GYNVYVDGTKVNEEL

SEQ ID NO: 52 - protein, 15 aa, Ruminococcus torques
YNVYVDGTKVNEELI

SEQ ID NO: 53 - protein, 15 aa, Ruminococcus torques
NVYVDGTKVNEELIA

SEQ ID NO: 54 - protein, 15 aa, Ruminococcus torques
VYVDGTKVNEELIAE

SEQ ID NO: 55 - protein, 15 aa, Ruminococcus torques
YVDGTKVNEELIAET

SEQ ID NO: 56 - protein, 15 aa, Ruminococcus torques
VDGTKVNEELIAETT

SEQ ID NO: 57 - protein, 15 aa, Ruminococcus torques
DGTKVNEELIAETTY

SEQ ID NO: 58 - protein, 15 aa, Ruminococcus torques
GTKVNEELIAETTYN

SEQ ID NO: 59 - protein, 15 aa, Ruminococcus torques
TKVNEELIAETTYNV

SEQ ID NO: 60 - protein, 15 aa, Ruminococcus torques
KVNEELIAETTYNVS

SEQ ID NO: 61 - protein, 15 aa, Ruminococcus torques
VNEELIAETTYNVSS

SEQ ID NO: 62 - protein, 15 aa, Ruminococcus torques
NEELIAETTYNVSSL

SEQ ID NO: 63 - protein, 15 aa, Ruminococcus torques
EELIAETTYNVSSLK

SEQ ID NO: 64 - protein, 15 aa, Ruminococcus torques
ELIAETTYNVSSLKD

SEQ ID NO: 65 - protein, 15 aa, Ruminococcus torques
LIAETTYNVSSLKDG

SEQ ID NO: 66 - protein, 15 aa, Ruminococcus torques
IAETTYNVSSLKDGT

SEQ ID NO: 67 - protein, 15 aa, Ruminococcus torques
AETTYNVSSLKDGTT

SEQ ID NO: 68 - protein, 15 aa, Ruminococcus torques
ETTYNVSSLKDGTTY

SEQ ID NO: 69 - protein, 15 aa, Ruminococcus torques
TTYNVSSLKDGTTYS

SEQ ID NO: 70 - protein, 15 aa, Ruminococcus torques
TYNVSSLKDGTTYSV

SEQ ID NO: 71 - protein, 15 aa, Ruminococcus torques
YNVSSLKDGTTYSVE

SEQ ID NO: 72 - protein, 15 aa, Ruminococcus torques
NVSSLKDGTTYSVEV

SEQ ID NO: 73 - protein, 15 aa, Ruminococcus torques
VSSLKDGTTYSVEVT

SEQ ID NO: 74 - protein, 15 aa, Ruminococcus torques
SSLKDGTTYSVEVTA

SEQ ID NO: 75 - protein, 15 aa, Ruminococcus torques
SLKDGTTYSVEVTAV

SEQ ID NO: 76 - protein, 15 aa, Ruminococcus torques
LKDGTTYSVEVTAVD

SEQ ID NO: 77 - protein, 15 aa, Ruminococcus torques
KDGTTYSVEVTAVDA

SEQ ID NO: 78 - protein, 15 aa, Ruminococcus torques
DGTTYSVEVTAVDAA

SEQ ID NO: 79 - protein, 15 aa, Ruminococcus torques
GTTYSVEVTAVDAAG

SEQ ID NO: 80 - protein, 15 aa, Ruminococcus torques
TTYSVEVTAVDAAGE

SEQ ID NO: 81 - protein, 15 aa, Ruminococcus torques
TYSVEVTAVDAAGEE

SEQ ID NO: 82 - protein, 15 aa, Ruminococcus torques
YSVEVTAVDAAGEES

SEQ ID NO: 83 - protein, 15 aa, Ruminococcus torques
SVEVTAVDAAGEESA

SEQ ID NO: 84 - protein, 15 aa, Ruminococcus torques
VEVTAVDAAGEESAK

SEQ ID NO: 85 - protein, 15 aa, Ruminococcus torques
EVTAVDAAGEESAKS

SEQ ID NO: 86 - protein, 15 aa, Ruminococcus torques
VTAVDAAGEESAKSE

SEQ ID NO: 87 - protein, 15 aa, Ruminococcus torques
TAVDAAGEESAKSEK

SEQ ID NO: 88 - protein, 15 aa, Ruminococcus torques
AVDAAGEESAKSEKV

SEQ ID NO: 89 - protein, 15 aa, Ruminococcus torques
VDAAGEESAKSEKVE

SEQ ID NO: 90 - protein, 15 aa, Ruminococcus torques
DAAGEESAKSEKVEF

SEQ ID NO: 91 - protein, 15 aa, Ruminococcus torques
AAGEESAKSEKVEFT

SEQ ID NO: 92 - protein, 15 aa, Ruminococcus torques
AGEESAKSEKVEFTT

SEQ ID NO: 93 - protein, 15 aa, Ruminococcus torques
GEESAKSEKVEFTTV

SEQ ID NO: 94 - protein, 15 aa, Ruminococcus torques
EESAKSEKVEFTTVK

SEQ ID NO: 95 - protein, 15 aa, Ruminococcus torques
ESAKSEKVEFTTVKK

SEQ ID NO: 96 - protein, 15 aa, Ruminococcus torques
APVDVKVSEITETSA

SEQ ID NO: 97 - protein, 15 aa, Ruminococcus torques
PVDVKVSEITETSAK

SEQ ID NO: 98 - protein, 15 aa, Ruminococcus torques
VDVKVSEITETSAKA

SEQ ID NO: 99 - protein, 15 aa, Ruminococcus torques
DVKVSEITETSAKAS

SEQ ID NO: 100 - protein, 15 aa, Ruminococcus torques
VKVSEITETSAKASW

SEQ ID NO: 101 - protein, 15 aa, Ruminococcus torques
KVSEITETSAKASWK

SEQ ID NO: 102 - protein, 15 aa, Ruminococcus torques
VSEITETSAKASWKN

SEQ ID NO: 103 - protein, 15 aa, Ruminococcus torques
SEITETSAKASWKNA

SEQ ID NO: 104 - protein, 15 aa, Ruminococcus torques
EITETSAKASWKNAA

SEQ ID NO: 105 - protein, 15 aa, Ruminococcus torques
ITETSAKASWKNAAD

SEQ ID NO: 106 - protein, 15 aa, Ruminococcus torques
TETSAKASWKNAADG

SEQ ID NO: 107 - protein, 15 aa, Ruminococcus torques
ETSAKASWKNAADGK

SEQ ID NO: 108 - protein, 15 aa, Ruminococcus torques
TSAKASWKNAADGKE

SEQ ID NO: 109 - protein, 15 aa, Ruminococcus torques
SAKASWKNAADGKEA

SEQ ID NO: 110 - protein, 15 aa, Ruminococcus torques
AKASWKNAADGKEAA

SEQ ID NO: 111 - protein, 15 aa, Ruminococcus torques
KASWKNAADGKEAAG

SEQ ID NO: 112 - protein, 15 aa, Ruminococcus torques
ASWKNAADGKEAAGY

SEQ ID NO: 113 - protein, 15 aa, Ruminococcus torques
SWKNAADGKEAAGYN

SEQ ID NO: 114 - protein, 15 aa, Ruminococcus torques
WKNAADGKEAAGYNV

SEQ ID NO: 115 - protein, 15 aa, Ruminococcus torques
KNAADGKEAAGYNVY

SEQ ID NO: 116 - protein, 15 aa, Ruminococcus torques
NAADGKEAAGYNVYV

SEQ ID NO: 117 - protein, 15 aa, Ruminococcus torques
AADGKEAAGYNVYVD

SEQ ID NO: 118 - protein, 15 aa, Ruminococcus torques
ADGKEAAGYNVYVDG

SEQ ID NO: 119 - protein, 15 aa, Ruminococcus torques
DGKEAAGYNVYVDGV

SEQ ID NO: 120 - protein, 15 aa, Ruminococcus torques
GKEAAGYNVYVDGVK

SEQ ID NO: 121 - protein, 15 aa, Ruminococcus torques
KEAAGYNVYVDGVKL

SEQ ID NO: 122 - protein, 15 aa, Ruminococcus torques
EAAGYNVYVDGVKLN

SEQ ID NO: 123 - protein, 15 aa, Ruminococcus torques
AAGYNVYVDGVKLNK

SEQ ID NO: 124 - protein, 15 aa, Ruminococcus torques
AGYNVYVDGVKLNKE

SEQ ID NO: 125 - protein, 15 aa, Ruminococcus torques
GYNVYVDGVKLNKEL

SEQ ID NO: 126 - protein, 15 aa, Ruminococcus torques
YNVYVDGVKLNKELL

SEQ ID NO: 127 - protein, 15 aa, Ruminococcus torques
NVYVDGVKLNKELLT

SEQ ID NO: 128 - protein, 15 aa, Ruminococcus torques
VYVDGVKLNKELLTE

SEQ ID NO: 129 - protein, 15 aa, Ruminococcus torques
YVDGVKLNKELLTEA

SEQ ID NO: 130 - protein, 15 aa, Ruminococcus torques
VDGVKLNKELLTEAS

SEQ ID NO: 131 - protein, 15 aa, Ruminococcus torques
DGVKLNKELLTEASC

SEQ ID NO: 132 - protein, 15 aa, Ruminococcus torques
GVKLNKELLTEASCG

SEQ ID NO: 133 - protein, 15 aa, Ruminococcus torques
VKLNKELLTEASCGL

SEQ ID NO: 134 - protein, 15 aa, Ruminococcus torques
KLNKELLTEASCGLT

SEQ ID NO: 135 - protein, 15 aa, Ruminococcus torques
LNKELLTEASCGLTN

SEQ ID NO: 136 - protein, 15 aa, Ruminococcus torques
NKELLTEASCGLTNL

SEQ ID NO: 137 - protein, 15 aa, Ruminococcus torques
KELLTEASCGLTNLK

SEQ ID NO: 138 - protein, 15 aa, Ruminococcus torques
ELLTEASCGLTNLKA

SEQ ID NO: 139 - protein, 15 aa, Ruminococcus torques
LLTEASCGLTNLKAE

SEQ ID NO: 140 - protein, 15 aa, Ruminococcus torques
LTEASCGLTNLKAET

SEQ ID NO: 141 - protein, 15 aa, Ruminococcus torques
TEASCGLTNLKAETT

SEQ ID NO: 142 - protein, 15 aa, Ruminococcus torques
EASCGLTNLKAETTY

SEQ ID NO: 143 - protein, 15 aa, Ruminococcus torques
ASCGLTNLKAETTYF

SEQ ID NO: 144 - protein, 15 aa, Ruminococcus torques
SCGLTNLKAETTYFV

SEQ ID NO: 145 - protein, 15 aa, Ruminococcus torques
CGLTNLKAETTYFVE

SEQ ID NO: 146 - protein, 15 aa, Ruminococcus torques
GLTNLKAETTYFVEV

SEQ ID NO: 147 - protein, 15 aa, Ruminococcus torques
LTNLKAETTYFVEVT

SEQ ID NO: 148 - protein, 15 aa, Ruminococcus torques
TNLKAETTYFVEVTA

SEQ ID NO: 149 - protein, 15 aa, Ruminococcus torques
NLKAETTYFVEVTAV

SEQ ID NO: 150 - protein, 15 aa, Ruminococcus torques
LKAETTYFVEVTAVD

SEQ ID NO: 151 - protein, 15 aa, Ruminococcus torques
KAETTYFVEVTAVDA

SEQ ID NO: 152 - protein, 15 aa, Ruminococcus torques
AETTYFVEVTAVDAA

SEQ ID NO: 153 - protein, 15 aa, Ruminococcus torques
ETTYFVEVTAVDAAG

SEQ ID NO: 154 - protein, 15 aa, Ruminococcus torques
TTYFVEVTAVDAAGN

SEQ ID NO: 155 - protein, 15 aa, Ruminococcus torques
TYFVEVTAVDAAGNE

SEQ ID NO: 156 - protein, 15 aa, Ruminococcus torques
YFVEVTAVDAAGNES

SEQ ID NO: 157 - protein, 15 aa, Ruminococcus torques
FVEVTAVDAAGNESV

SEQ ID NO: 158 - protein, 15 aa, Ruminococcus torques
VEVTAVDAAGNESVK

SEQ ID NO: 159 - protein, 15 aa, Ruminococcus torques
EVTAVDAAGNESVKS

SEQ ID NO: 160 - protein, 15 aa, Ruminococcus torques
VTAVDAAGNESVKSE

SEQ ID NO: 161 - protein, 15 aa, Ruminococcus torques
TAVDAAGNESVKSEK

SEQ ID NO: 162 - protein, 15 aa, Ruminococcus torques
AVDAAGNESVKSEKV

SEQ ID NO: 163 - protein, 15 aa, Ruminococcus torques
VDAAGNESVKSEKVT

SEQ ID NO: 164 - protein, 15 aa, Ruminococcus torques
DAAGNESVKSEKVTF

SEQ ID NO: 165 - protein, 15 aa, Ruminococcus torques
AAGNESVKSEKVTFK

SEQ ID NO: 166 - protein, 15 aa, Ruminococcus torques
AGNESVKSEKVTFKT

SEQ ID NO: 167 - protein, 15 aa, Ruminococcus torques
GNESVKSEKVTFKTL

SEQ ID NO: 168 - protein, 15 aa, Ruminococcus torques
NESVKSEKVTFKTLK

SEQ ID NO: 169 - protein, 19 aa, Ruminococcus torques
ETSAKVSWKNAADGKEAAG

SEQ ID NO: 170 - protein, 19 aa, Artificial
  Alanine scanning sequence
  misc_feature (1)..(19): Alanine scanning sequence of RUCILP1
ETSAKVSWKNAADGKEAAA

SEQ ID NO: 171 - protein, 19 aa, Artificial
  Alanine scanning sequence
  misc_feature (1)..(19): Alanine scanning sequence of RUCILP1
ETSAKVSWKNAADGKEAGG

SEQ ID NO: 172 - protein, 19 aa, Artificial
  Alanine scanning sequence
  misc_feature (1)..(19): Alanine scanning sequence of RUCILP1
ETSAKVSWKNAADGKEGAG

SEQ ID NO: 173 - protein, 19 aa, Artificial
  Alanine scanning sequence
  misc_feature (1)..(19): Alanine scanning sequence of RUCILP1
ETSAKVSWKNAADGKAAAG

SEQ ID NO: 174 - protein, 19 aa, Artificial
  Alanine scanning sequence
  misc_feature (1)..(19): Alanine scanning sequence of RUCILP1
ETSAKVSWKNAADGAEAAG

SEQ ID NO: 175 - protein, 19 aa, Artificial
  Alanine scanning sequence
  misc_feature (1)..(19): Alanine scanning sequence of RUCILP1
ETSAKVSWKNAADAKEAAG

SEQ ID NO: 176 - protein, 19 aa, Artificial
  Alanine scanning sequence
  misc_feature (1)..(19): Alanine scanning sequence of RUCILP1
ETSAKVSWKNAAAGKEAAG

SEQ ID NO: 177 - protein, 19 aa, Artificial
  Alanine scanning sequence
  misc_feature (1)..(19): Alanine scanning sequence of RUCILP1
ETSAKVSWKNAGDGKEAAG

SEQ ID NO: 178 - protein, 19 aa, Artificial
  Alanine scanning sequence
  misc_feature (1)..(19): Alanine scanning sequence of RUCILP1
ETSAKVSWKNGADGKEAAG

SEQ ID NO: 179 - protein, 19 aa, Artificial
  Alanine scanning sequence
  misc_feature (1)..(19): Alanine scanning sequence of RUCILP1
ETSAKVSWKAAADGKEAAG

SEQ ID NO: 180 - protein, 19 aa, Artificial
  Alanine scanning sequence
  misc_feature (1)..(19): Alanine scanning sequence of RUCILP1
ETSAKVSWANAADGKEAAG

SEQ ID NO: 181 - protein, 19 aa, Artificial
  Alanine scanning sequence
  misc_feature (1)..(19): Alanine scanning sequence of RUCILP1
ETSAKVSAKNAADGKEAAG

SEQ ID NO: 182 - protein, 19 aa, Artificial
  Alanine scanning sequence
  misc_feature (1)..(19): Alanine scanning sequence of RUCILP1
ETSAKVAWKNAADGKEAAG

SEQ ID NO: 183 - protein, 19 aa, Artificial
  Alanine scanning sequence
  misc_feature (1)..(19): Alanine scanning sequence of RUCILP1
ETSAKASWKNAADGKEAAG

SEQ ID NO: 184 - protein, 19 aa, Artificial
  Alanine scanning sequence
  misc_feature (1)..(19): Alanine scanning sequence of RUCILP1
ETSAAVSWKNAADGKEAAG

SEQ ID NO: 185 - protein, 19 aa, Artificial
  Alanine scanning sequence
  misc_feature (1)..(19): Alanine scanning sequence of RUCILP1
ETSGKVSWKNAADGKEAAG

SEQ ID NO: 186 - protein, 19 aa, Artificial
  Alanine scanning sequence
  misc_feature (1)..(19): Alanine scanning sequence of RUCILP1
ETAAKVSWKNAADGKEAAG

SEQ ID NO: 187 - protein, 19 aa, Artificial
  Alanine scanning sequence
  misc_feature (1)..(19): Alanine scanning sequence of RUCILP1
EASAKVSWKNAADGKEAAG

SEQ ID NO: 188 - protein, 19 aa, Artificial
  Alanine scanning sequence
  misc_feature (1)..(19): Alanine scanning sequence of RUCILP1
ATSAKVSWKNAADGKEAAG

SEQ ID NO: 189 - protein, 19 aa, Ruminococcus torques
ETSAKASWKNAADGKEAAG

SEQ ID NO: 190 - protein, 19 aa, Artificial
  Alanine scanning sequence
  misc_feature (1)..(19): Alanine scanning sequence of RUCILP2
ETSAKASWKNAADGKEAAA

SEQ ID NO: 191 - protein, 19 aa, Artificial
  Alanine scanning sequence
  misc_feature (1)..(19): Alanine scanning sequence of RUCILP2
ETSAKASWKNAADGKEAGG

SEQ ID NO: 192 - protein, 19 aa, Artificial
  Alanine scanning sequence
  misc_feature (1)..(19): Alanine scanning sequence of RUCILP2
ETSAKASWKNAADGKEGAG

SEQ ID NO: 193 - protein, 19 aa, Artificial
  Alanine scanning sequence
  misc_feature (1)..(19): Alanine scanning sequence of RUCILP2
ETSAKASWKNAADGKAAAG

SEQ ID NO: 194 - protein, 19 aa, Artificial
  Alanine scanning sequence
  misc_feature (1)..(19): Alanine scanning sequence of RUCILP2
ETSAKASWKNAADGAEAAG

SEQ ID NO: 195 - protein, 19 aa, Artificial
  Alanine scanning sequence
  misc_feature (1)..(19): Alanine scanning sequence of RUCILP2
ETSAKASWKNAADAKEAAG

SEQ ID NO: 196 - protein, 19 aa, Artificial
  Alanine scanning sequence
  misc_feature (1)..(19): Alanine scanning sequence of RUCILP2
ETSAKASWKNAAAGKEAAG

SEQ ID NO: 197 - protein, 19 aa, Artificial
  Alanine scanning sequence
  misc_feature (1)..(19): Alanine scanning sequence of RUCILP2
ETSAKASWKNAGDGKEAAG

SEQ ID NO: 198 - protein, 19 aa, Artificial
  Alanine scanning sequence
  misc_feature (1)..(19): Alanine scanning sequence of RUCILP2
ETSAKASWKNGADGKEAAG

SEQ ID NO: 199 - protein, 19 aa, Artificial
  Alanine scanning sequence
  misc_feature (1)..(19): Alanine scanning sequence of RUCILP2
ETSAKASWKAAADGKEAAG

SEQ ID NO: 200 - protein, 19 aa, Artificial
  Alanine scanning sequence
  misc_feature (1)..(19): Alanine scanning sequence of RUCILP2
ETSAKASWANAADGKEAAG

SEQ ID NO: 201 - protein, 19 aa, Artificial
  Alanine scanning sequence
  misc_feature (1)..(19): Alanine scanning sequence of RUCILP2
ETSAKASAKNAADGKEAAG

SEQ ID NO: 202 - protein, 19 aa, Artificial
  Alanine scanning sequence
  misc_feature (1)..(19): Alanine scanning sequence of RUCILP2
ETSAKAAWKNAADGKEAAG

SEQ ID NO: 203 - protein, 19 aa, Artificial
  Alanine scanning sequence
  misc_feature (1)..(19): Alanine scanning sequence of RUCILP2
ETSAKGSWKNAADGKEAAG

SEQ ID NO: 204 - protein, 19 aa, Artificial
  Alanine scanning sequence
  misc_feature (1)..(19): Alanine scanning sequence of RUCILP2
ETSAAASWKNAADGKEAAG

SEQ ID NO: 205 - protein, 19 aa, Artificial
  Alanine scanning sequence
  misc_feature (1)..(19): Alanine scanning sequence of RUCILP2
ETSGKASWKNAADGKEAAG

SEQ ID NO: 206 - protein, 19 aa, Artificial
  Alanine scanning sequence
  misc_feature (1)..(19): Alanine scanning sequence of RUCILP2
ETAAKASWKNAADGKEAAG

SEQ ID NO: 207 - protein, 19 aa, Artificial
  Alanine scanning sequence
  misc_feature (1)..(19): Alanine scanning sequence of RUCILP2
EASAKASWKNAADGKEAAG

SEQ ID NO: 208 - protein, 19 aa, Artificial
  Alanine scanning sequence
  misc_feature (1)..(19): Alanine scanning sequence of RUCILP2
ATSAKASWKNAADGKEAAG

SEQ ID NO: 209 - protein, 19 aa, Ruminococcus torques
ETSAKVSWKNAADGKEAAG

SEQ ID NO: 210 - protein, 18 aa, Ruminococcus torques
TSAKVSWKNAADGKEAAG

SEQ ID NO: 211 - protein, 17 aa, Ruminococcus torques
SAKVSWKNAADGKEAAG

SEQ ID NO: 212 - protein, 16 aa, Ruminococcus torques
AKVSWKNAADGKEAAG

SEQ ID NO: 213 - protein, 15 aa, Ruminococcus torques
KVSWKNAADGKEAAG

SEQ ID NO: 214 - protein, 14 aa, Ruminococcus torques
VSWKNAADGKEAAG

SEQ ID NO: 215 - protein, 13 aa, Ruminococcus torques
SWKNAADGKEAAG

SEQ ID NO: 216 - protein, 12 aa, Ruminococcus torques
WKNAADGKEAAG

SEQ ID NO: 217 - protein, 11 aa, Ruminococcus torques
KNAADGKEAAG

SEQ ID NO: 218 - protein, 10 aa, Ruminococcus torques
NAADGKEAAG

SEQ ID NO: 219 - protein, 9 aa, Ruminococcus torques
AADGKEAAG

SEQ ID NO: 220 - protein, 8 aa, Ruminococcus torques
ADGKEAAG

SEQ ID NO: 221 - protein, 7 aa, Ruminococcus torques
DGKEAAG

SEQ ID NO: 222 - protein, 6 aa, Ruminococcus torques
GKEAAG

SEQ ID NO: 223 - protein, 5 aa, Ruminococcus torques
KEAAG

SEQ ID NO: 224 - protein, 4 aa, Ruminococcus torques
EAAG

SEQ ID NO: 225 - protein, 3 aa, Ruminococcus torques
AAG

SEQ ID NO: 226 - protein, 18 aa, Ruminococcus torques
ETSAKVSWKNAADGKEAA

SEQ ID NO: 227 - protein, 17 aa, Ruminococcus torques
ETSAKVSWKNAADGKEA

SEQ ID NO: 228 - protein, 16 aa, Ruminococcus torques
ETSAKVSWKNAADGKE

SEQ ID NO: 229 - protein, 15 aa, Ruminococcus torques
ETSAKVSWKNAADGK

SEQ ID NO: 230 - protein, 14 aa, Ruminococcus torques
ETSAKVSWKNAADG

SEQ ID NO: 231 - protein, 13 aa, Ruminococcus torques
ETSAKVSWKNAAD

SEQ ID NO: 232 - protein, 12 aa, Ruminococcus torques
ETSAKVSWKNAA

SEQ ID NO: 233 - protein, 11 aa, Ruminococcus torques
ETSAKVSWKNA

SEQ ID NO: 234 - protein, 10 aa, Ruminococcus torques
ETSAKVSWKN

SEQ ID NO: 235 - protein, 9 aa, Ruminococcus torques
ETSAKVSWK

SEQ ID NO: 236 - protein, 8 aa, Ruminococcus torques
ETSAKVSW

SEQ ID NO: 237 - protein, 7 aa, Ruminococcus torques
ETSAKVS

SEQ ID NO: 238 - protein, 6 aa, Ruminococcus torques
ETSAKV

SEQ ID NO: 239 - protein, 5 aa, Ruminococcus torques
ETSAK

SEQ ID NO: 240 - protein, 4 aa, Ruminococcus torques
ETSA

SEQ ID NO: 241 - protein, 3 aa, Ruminococcus torques
ETS

SEQ ID NO: 242 - protein, 19 aa, Ruminococcus torques
ETSAKASWKNAADGKEAAG

SEQ ID NO: 243 - protein, 18 aa, Ruminococcus torques
TSAKASWKNAADGKEAAG

SEQ ID NO: 244 - protein, 17 aa, Ruminococcus torques
SAKASWKNAADGKEAAG

SEQ ID NO: 245 - protein, 16 aa, Ruminococcus torques
AKASWKNAADGKEAAG

SEQ ID NO: 246 - protein, 15 aa, Ruminococcus torques
KASWKNAADGKEAAG

SEQ ID NO: 247 - protein, 14 aa, Ruminococcus torques
ASWKNAADGKEAAG

SEQ ID NO: 248 - protein, 13 aa, Ruminococcus torques
SWKNAADGKEAAG

SEQ ID NO: 249 - protein, 12 aa, Ruminococcus torques
WKNAADGKEAAG

SEQ ID NO: 250 - protein, 11 aa, Ruminococcus torques
KNAADGKEAAG

SEQ ID NO: 251 - protein, 10 aa, Ruminococcus torques
NAADGKEAAG

SEQ ID NO: 252 - protein, 9 aa, Ruminococcus torques
AADGKEAAG

SEQ ID NO: 253 - protein, 8 aa, Ruminococcus torques
ADGKEAAG

SEQ ID NO: 254 - protein, 7 aa, Ruminococcus torques
DGKEAAG

SEQ ID NO: 255 - protein, 6 aa, Ruminococcus torques
GKEAAG

SEQ ID NO: 256 - protein, 5 aa, Ruminococcus torques
KEAAG

SEQ ID NO: 257 - protein, 4 aa, Ruminococcus torques
EAAG

SEQ ID NO: 258 - protein, 3 aa, Ruminococcus torques
AAG

SEQ ID NO: 259 - protein, 18 aa, Ruminococcus torques
ETSAKASWKNAADGKEAA

SEQ ID NO: 260 - protein, 17 aa, Ruminococcus torques
ETSAKASWKNAADGKEA

SEQ ID NO: 261 - protein, 16 aa, Ruminococcus torques
ETSAKASWKNAADGKE

SEQ ID NO: 262 - protein, 15 aa, Ruminococcus torques
ETSAKASWKNAADGK

SEQ ID NO: 263 - protein, 14 aa, Ruminococcus torques
ETSAKASWKNAADG

SEQ ID NO: 264 - protein, 13 aa, Ruminococcus torques
ETSAKASWKNAAD

SEQ ID NO: 265 - protein, 12 aa, Ruminococcus torques
ETSAKASWKNAA

SEQ ID NO: 266 - protein, 11 aa, Ruminococcus torques
ETSAKASWKNA

SEQ ID NO: 267 - protein, 10 aa, Ruminococcus torques
ETSAKASWKN

SEQ ID NO: 268 - protein, 9 aa, Ruminococcus torques
ETSAKASWK

SEQ ID NO: 269 - protein, 8 aa, Ruminococcus torques
ETSAKASW

SEQ ID NO: 270 - protein, 7 aa, Ruminococcus torques
ETSAKAS

SEQ ID NO: 271 - protein, 6 aa, Ruminococcus torques
ETSAKA

SEQ ID NO: 272 - protein, 5 aa, Ruminococcus torques
ETSAK

SEQ ID NO: 273 - protein, 4 aa, Ruminococcus torques
ETSA

SEQ ID NO: 274 - protein, 3 aa, Ruminococcus torques
ETS

(19) United States
(12) Patent Application Publication
(10) Pub. No.: US 2024/0336661 A1
(43) Pub. Date: Oct. 10, 2024

(54) PEPTIDES DERIVED FROM RUMINOCOCCUS TORQUES

(71) Applicant: University of Copenhagen, Copenhagen K (DK)

(72) Inventors: Oluf Pedersen, Hotel (DK); Yong Fan, Kongens Lyngby (DK)

(21) Appl. No.: 18/564,398

(22) PCT Filed: Jun. 2, 2022
(86) PCT No.: PCT/EP2022/065070

§ 371 Date: Nov. 27, 2023

(30) Foreign Application Priority Data
Jun. 3, 2021 (EP) 21177546.5
Jan. 27, 2022 (EP) 22153678.2
Jan. 28, 2022 (EP) 22153847.3

(51) Int. Cl. C07K 14/195; A61K 38/00; A61P 3/04; A61P 3/10; A61P 19/00

(52) U.S. Cl. CPC C07K 14/195; A61P 3/04; A61P 3/10; A61P 19/00; A61K 38/00

(57) ABSTRACT

The present invention relates to polypeptides derived from Ruminococcus torques, and polypeptide fragments and variants thereof useful for treatment and/or prevention of metabolic disorders, muscle disorders and injuries, and bone disorders, and host cells comprising said polypeptides, polypeptide fragments or variants thereof for use as a probiotic or as a Live Biopharmaceutical Product (LBP).

### TECHNICAL FIELD

The present invention relates to polypeptides derived from Ruminococcus torques, and polypeptide fragments and variants thereof useful for treatment and/or prevention of metabolic disorders, muscle disorders and injuries, and bone disorders, and host cells comprising said polypeptides, polypeptide fragments or variants thereof for use as a probiotic or as a Live Biopharmaceutical Product.

### BACKGROUND

From the outcome of the past 10 years' epidemiological, physiological, ecological and omics-based human studies, complemented by cellular studies and mechanistic experiments in animals, it appears that microbial communities mediate a considerable part of the environmental influence on human health^{1, 2}. These non-pathogenic (i.e., commensal and mutualistic) microorganisms, collectively referred to as the microbiota, include a vast number of interacting bacteria, archaea, bacteriophages, eukaryotic virus and fungi coexisting on human surfaces and in all body cavities. The collection of all microbial genes (that is, the microbiome) in and on an individual represents a genetic repertoire that is more than one order of magnitude higher in genes than the human nuclear genome³.

The majority of microorganisms that inhabit humans reside within distal gut where they have important roles in training of host immunity, digesting food, regulating gut endocrine function and neurological signaling, modifying drug action and metabolism, eliminating toxins and in releasing several microbial compounds that influence the host^{1, 2}. There are many examples of human gut bacterial strains. One of them is Ruminococcus torques. In human metagenomes, the contribution of certain strains of R. torques can reach up to 10% of the total relative abundance.⁸ The bacteria has been associated with mucosa, and can degrade mucus.

A prime example of one of the few bacterial compounds known to regulating host metabolism is Amuc_1100, a protein residing in the outer membrane of the commensal gut bacterium Akkermansia muciniphila⁴. The bacterium has in preclinical experiments when administered either in live or pasteurized forms been linked with improved metabolism⁴. In a recent human pilot intervention, prescription of A. muciniphila was shown to be tolerable, without adverse effects and to alleviate dys-metabolism of overweight and obese patients with a magnitude of clinical relevance⁵.

According to the World Health Organization (WHO) obesity has tripled since 1975; in 2016, more than 1.9 billion adults were overweight and of these, over 650 million were obese. Obesity is closely linked to other conditions, such as metabolic syndrome with indications including high blood pressure, fatty liver disease (FLD), and type 2 diabetes (T2D). WHO estimates that the amount of people with diabetes has quadrupled since 1980. Today, 422 million people worldwide have diabetes, whereof a majority suffer from T2D caused by overweight, obesity and lack of physical activity.

Other diseases affecting a large number of people include muscular and skeletal diseases, disorders and injuries. Bone loss, or weak bones, is a common disorder, especially in countries with an ageing population. In some countries, osteoporosis affects up to 70% of people over 80 years. Several muscular disorders, hereunder neuromuscular disorders such as muscular dystrophy, causes weakness and breakdown of skeletal muscles over time. The prognosis of muscular dystrophy and other neuromuscular disorders ranges from mild to severe depending on the specific cause.

Thus, there is clearly a need in the art for improved treatment and prevention of the diseases and disorders listed above. An approach to such treatment may be to identify organisms and compounds derived from the gut microbiome that promote health via positive impact on host metabolism.

### SUMMARY

The present invention relates to a polypeptide and use thereof for treatment and/or prevention of metabolic, muscle and bone disorders, and host cells comprising said polypeptides, polypeptide fragments and variants thereof for use as a probiotic or as a Live Biopharmaceutical Product (LBP).

The inventors have shown that bacterial peptides derived from Ruminococcus torques, as well as fragments and variants of said peptides, are likely effective in the treatment and prevention of metabolic disorders, muscle disorders and injuries, and bone disorders.

Thus, provided herein is an isolated polypeptide having a length of less than 200 amino acids comprising or consisting of an amino acid sequence selected from the group consisting of:
a) the amino acid sequence according to SEQ ID NO: 4 and/or SEQ ID NO: 19;
b) a variant of SEQ ID NO: 4 and/or SEQ ID NO: 19, wherein said variant has at least 60%, such as at least 70%, such as at least 75%, such as at least 80%, such as at least 85%, such as at least 90%, such as at least 95% sequence identity to SEQ ID NO: 4 and/or SEQ ID NO: 19, but less than 99% sequence identity to SEQ ID NO: 4 and/or SEQ ID NO: 19;
c) a variant of SEQ ID NO: 4 and/or SEQ ID NO: 19, wherein said variant has between 1 and 40 amino acid substitutions relative to SEQ ID NO: 4 and/or SEQ ID NO: 19, such as 5, 10, 15, 20, 25, 30, or 35 amino acid substitutions relative to SEQ ID NO: 4 and/or SEQ ID NO: 19;
d) a fragment of SEQ ID NO: 4 and/or SEQ ID NO: 19 having a length of at least 10 amino acids, or a variant of said fragment having between 1 and 5 amino acid substitutions relative to SEQ ID NO: 4 and/or SEQ ID NO: 19, respectively, such as 1, 2 or 3 amino acid substitutions relative to SEQ ID NO: 4 and/or SEQ ID NO: 19, wherein said polypeptide has a length of less than 50 amino acids;
e) an amino acid sequence differing from SEQ ID NO: 4 and/or SEQ ID NO: 19 by truncation at the N-terminus by at least one amino acid, such as between 1-67 amino acids, such as between 1-60 amino acids, for example between 1-50 amino acids, such as between 1-40 amino acids, for example between 1-30 amino acids, such as between 1-20 amino acids, for example between 1-10 amino acids, such as between 1-5 amino acids, or a variant thereof having between 1 and 10 amino acid substitutions relative to SEQ ID NO: 4 and/or SEQ ID NO: 19, such as 1, 2, 3, 4, 5, 6, 7, 8 or 9 amino acid substitutions relative to SEQ ID NO: 4 and/or SEQ ID NO: 19;
f) an amino acid sequence differing from SEQ ID NO: 4 and/or SEQ ID NO: 19 by truncation at the C-terminus by at least one amino acid, such as between 1-21 amino acids, such as between 1-20 amino acids, for example between 1-15 amino acids, such as between 1-10 amino acids, for example between 1-5 amino acids, or a variant thereof having between 1 and 30 amino acid substitutions relative to SEQ ID NO: 4 and/or SEQ ID NO: 19, such as 1, 5, 10, 15, 20 or 25 amino acid substitutions relative to SEQ ID NO: 4 and/or SEQ ID NO: 19;
g) an amino acid sequence differing from SEQ ID NO: 4 and/or SEQ ID NO: 19 by truncation at the N-terminus by at least one amino acid, 1-67 amino acids, such as between 1-60 amino acids, for example between 1-50 amino acids, such as between 1-40 amino acids, for example between 1-30 amino acids, such as between 1-20 amino acids, for example between 1-10 amino acids, such as between 1-5 amino acids, and at the C-terminus by at least one amino acid, such as between 1-21 amino acids, such as between 1-20 amino acids, for example between 1-15 amino acids, such as between 1-10 amino acids, for example between 1-5 amino acids, wherein said polypeptide has a length of at least 10 amino acids, or a variant thereof having between 1 and 5 amino acid substitutions relative to SEQ ID NO: 4 and/or SEQ ID NO: 19, such as 1, 2 or 3 amino acid substitutions relative to SEQ ID NO: 4 and/or SEQ ID NO: 19;
h) the amino acid sequence according to SEQ ID NO: 5 and/or SEQ ID NO: 20;
i) a variant of SEQ ID NO: 5 and/or SEQ ID NO: 20, wherein said variant has at least 70%, such as at least 75%, such as at least 80%, such as at least 85%, such as at least 90%, such as at least 95% sequence identity to SEQ ID NO: 5 and/or SEQ ID NO: 20, but less than 99% sequence identity to SEQ ID NO: 5 and/or SEQ ID NO: 20;
j) a variant of SEQ ID NO: 5 and/or SEQ ID NO: 20, wherein said variant has between 1 and 10 amino acid substitutions relative to SEQ ID NO: 5 and/or SEQ ID NO: 20, such as 1, 2, 3, 4, 5, 6, 7, 8, 9 or 10 amino acid substitutions relative to SEQ ID NO: 5 and/or SEQ ID NO: 20, wherein said polypeptide has a length of less than 50 amino acids;
k) a fragment of SEQ ID NO: 5 and/or SEQ ID NO: 20 comprising at least 10 consecutive amino acids of SEQ ID NO: 5 and/or SEQ ID NO: 20, or a variant thereof having between 1 and 5 amino acid substitutions relative to SEQ ID NO: 5 and/or SEQ ID NO: 20, such as 1, 2, 3, or 4 amino acid substitutions relative to SEQ ID NO: 5 and/or SEQ ID NO: 20, wherein said polypeptide has a length of less than 50 amino acids;
l) a fragment of SEQ ID NO: 19, wherein said fragment is selected from the group consisting of SEQ ID NOs: 27, 33, 34, 35, 36, 37 and 95, and respective variants thereof having between 1 and 3 amino acid substitutions relative to SEQ ID NO: 19, such as 1, 2 or 3 amino acid substitutions relative to SEQ ID NO: 19, wherein said polypeptide has a length of less than 50 amino acids;
m) a fragment of SEQ ID NO: 4, wherein said fragment is selected from the group consisting of SEQ ID NOs: 107, 108, 109, 110, 111, 165 and 168, and respective variants thereof having between 1 and 3 amino acid substitutions relative to SEQ ID NO: 4, such as 1, 2 or 3 amino acid substitutions relative to SEQ ID NO: 4, wherein said polypeptide has a length of less than 50 amino acids;
n) a fragment of a variant of SEQ ID NO: 19, wherein said fragment is selected from the group consisting of SEQ ID NOs: 173, 176, 181 and 188, and respective variants thereof having between 1 and 3 amino acid substitutions relative to SEQ ID NO: 19, such as 1, 2 or 3 amino acid substitutions relative to SEQ ID NO: 19, wherein said polypeptide has a length of less than 50 amino acids;
o) a fragment of a variant of SEQ ID NO: 4, wherein said fragment is selected from the group consisting of SEQ ID NOs: 193, 196, 201 and 208, and respective variants thereof having between 1 and 3 amino acid substitutions relative to SEQ ID NO: 4, such as 1, 2 or 3 amino acid substitutions relative to SEQ ID NO: 4, wherein said polypeptide has a length of less than 50 amino acids;
p) a fragment of SEQ ID NO: 19, wherein said fragment is selected from the group consisting of SEQ ID NOs: 210, 211, 212, 213, 229, 232, 233, 234 and 235, and respective variants thereof having between 1 and 3 amino acid substitutions relative to SEQ ID NO: 19, such as 1, 2 or 3 amino acid substitutions relative to SEQ ID NO: 19, wherein said polypeptide has a length of less than 50 amino acids; and
q) a fragment of SEQ ID NO: 4, wherein said fragment is selected from the group consisting of SEQ ID NOs: 243, 244, 245, 246, 262, 265, 266, 267 and 268, and respective variants thereof having between 1 and 3 amino acid substitutions relative to SEQ ID NO: 4, such as 1, 2 or 3 amino acid substitutions relative to SEQ ID NO: 4, wherein said polypeptide has a length of less than 50 amino acids.

Further provided herein is an isolated polynucleotide encoding the polypeptide of the present invention.

Also provided herein is a vector comprising the polynucleotide according to the present invention.

Further provided herein is a host cell comprising the polynucleotide and/or the vector according to the present invention.

Also provided herein is a pharmaceutical composition comprising the polypeptide, the polynucleotide, the vector, and/or the host cell according to the present invention.

Also provided herein is a dietary composition comprising the polypeptide, the conjugate, the polynucleotide, the vector, and/or the host cell according to the present invention, wherein the dietary composition comprises one or more of prebiotics, probiotics, Live Biopharmaceutical Products (LBPs), synbiotics, proteins, lipids, carbohydrates, vitamins, fibers, and/or nutrients, such as dietary minerals.

Further provided herein is a polypeptide, the conjugate, the polynucleotide, the vector, the host cell, and/or the pharmaceutical composition according to the present invention for use as a medicament.

Further provided herein is the host cell according to the present invention for use as a probiotic or as a Live Biopharmaceutical Product (LBP).

Also provided herein is the use of the polypeptide, the conjugate, the vector, and/or the host cell according to the present invention, as a food ingredient or as a food or beverage additive.

Further provided herein is the use of the host cell according to the present invention as a probiotic or as a Live Biopharmaceutical Product (LBP).

Also provided herein is the polypeptide, the conjugate, the polynucleotide, the vector, the host cell, and/or the pharmaceutical composition according to the present invention for use in the treatment and/or prevention of metabolic disorders, muscle disorders and injuries, and/or bone disorders.

Further provided herein is the use of the polypeptide, the conjugate, the vector, the host cell, and/or the pharmaceutical composition according to the present invention, in the manufacture of a medicament for treatment of metabolic disorders, muscle disorders and injuries, and/or bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and/or osteopetrosis.

Also provided herein is a method for the treatment of metabolic disorders, muscle disorders and injuries, and/or bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and/or osteopetrosis, wherein the method comprises administering the polypeptide, the conjugate, the polynucleotide, the vector, the host cell, and/or the pharmaceutical composition according to the present invention to an individual in need thereof.

### DESCRIPTION OF DRAWINGS

FIG. 1. Amino acid sequence alignments of human FNDC5, bacterial FNDC5-like protein, human irisin, and RUCILP2, respectively. Identical amino acid residues between RUCILP2 and human irisin are denoted by asterisks; low and high degrees of similarity are represented by a period and a colon, respectively. The multiple sequence alignments of amino acids in human FNDC5, bacterial FNDC5-like protein, human irisin, and RUCILP2, respectively, was performed using open access tool Clustal Omega (https://www.ebi.ac.uk/Tools/msa/clustalo/) to determine the number of identical and conserved residues. Thus, the figure shows the level of amino acid sequence similarities among human FNDC5, bacterial FNDC5-like protein, human irisin, and RUCILP2, respectively.

FIG. 2. Detection of dimerized RUCILP1 and RUCILP2 in culture medium. Left, Coomassie brilliant blue staining of proteins on polyvinylidene fluoride blot membrane indicated equal loading amount of proteins in each well. Right, Western blot against FNDC5 in medium from Ruminoccocus torgues (RT)-ATCC 27756 (n=3) and the control strain RT-ATCC 35915 (n=3), respectively, after anaerobic culture for 3 days with bacterial cell density of ˜10¹⁰ per ml culture medium. Thus, the figure results suggest that Ruminoccocus torgues (RT)-ATCC 27756 strain releases RUCILP2 in dimerized form to the growth culture medium.

FIG. 3. Predicted structure of RUCILP2 and alignment with irisin. The open-source software I-TASSER was used for protein structure modeling by iterative threading assembly simulations. The open access PyMOL (v2.1.1) tool was applied as a visualization tool for the predicted 3D structures. This figure suggests that RUCILP2 is a structural analogue to irisin.

FIG. 4. Docking models of interaction between integrin αV/β5 receptor and RUCILP2 (left panel) and irisin (right panel), respectively. The putative integrin-binding regions at amino acids 60-76 and 101-118, respectively, are shown in dark. The binding residues of RUCILP2 appear closer to the integrin αV/β5 receptor than those of irisin are. The binding abilities of RUCILP2 and irisin to the integrin αV/β5 receptor were assessed by Autodock (v4.2.6) computational analysis. The final complex structures of docking models were demonstrated by PyMOL (v2.1.1). The figure suggests binding between RUCILP2 and integrin αV/β5 receptor.

FIG. 5. Visualization of predicted binding sites in RUCILP2 to integrin αV/β5 receptor. The ZDOCK webserver ZDOCK (https://zdock.umassmed.edu/) was applied to predict the highest ranked model of RUCILP2 and integrin αV/β5 receptor complex. The model was visualized in the PyMOL (v2.1.1) program and showed binding at amino acids residues at V7, E9 and E58, respectively, of RUCILP2 to integrin αV/β5 receptor.

FIG. 6. Co-immunoprecipitation on a nickel ion column to validate binding of recombinant RUCILP2 to the αV/β5 integrin receptor complex. 100 nM Fc-fused RUCILP2 was incubated with 5 nM of His-tag αV/β5 integrin receptor, followed by immunoprecipitation using nickel-nitrilotriacetic acid (Ni-NTA) agarose. Precipitated integrins and co-precipitated irisin were analyzed by immuno-blot analysis. Elution indicates a mixture of disassociated integrin and RUCILP2 from the integrin-RUCILP2 complex. Before loading, samples were a mixture of co-incubation of RUCILP2 and integrin receptor. Thus, the experiment shows a direct interaction between RUCILP2 and integrin αV/β5.

FIG. 7. Application of a duplex RNAscope-based mRNA in situ hybridization array to identify signal dots of integrin αV/β5 receptor (ITGAV and ITGB5 mRNAs) in submucosa of normal human colon. The two target mRNAs were stained as red (ITGAV) and green (ITGB5) signal dots, respectively. The experimental setup included Polr2a (RNA polymerase II subunit A, red), and PPIB (Peptidylprolyl isomerase B, green) as positive control and DapB (Dihydrodipicolinate reductase) as negative control probe sets. Images were acquired using a 20× objective with a Zeiss AxioScan. The visualized signal demonstrates the presence of integrin αV/β5 receptor in human colon tissue.

FIG. 8. Duplex RNAscope-based mRNA in situ hybridization array to identify signal dots (submucosa) of integrin αV/β5 receptor (ITGAV and ITGB5 mRNAs) and cocaine- and amphetamine-regulated transcript protein (CART) in normal human colon. The two target mRNAs are stained as red (ITGAV and ITGB5) and green (CART) signal dots. The experimental setup included DapB (Dihydrodipicolinate reductase) as negative control probe sets. Images were acquired using a 20× objective with a Zeiss AxioScan.

FIG. 9. Exposure of recombinant RUCILP2 to human visceral white pre-adipocytes or murine inguinal white adipocytes causes up-regulation of the expression of genes involved in thermogenesis/browning. Upper panel: mRNA expression level of adipocyte differentiation marker genes including Ucp1, Pparγ1, Dio2, and Cox2 and brown adipocyte-selective genes including Cpt1b and Ebf2 on human white preadipocytes (HWP). Human white preadipocytes were cultured until 80% confluent and switched into differentiation media (with 0.3 ml/ml fetal calf serum, 8 ug/ml d-biotin, 0.5 ug/ml insulin, 400 ng/ml dexamethasone). The differentiation process to mature adipocytes was completed after 12-14 days. The treatment of RUCILP2 started from the third day of differentiation. Cells were harvested after 14 days of differentiation and the gene expressions were quantified by q-PCR. All the gene expression levels were normalized to the gene expression level of TATA-binding protein (TBP). Lower panel: stromal vascular fraction cells from murine inguinal white adipose tissue were differentiated into adipocytes and treated with indicated doses of recombinant RUCILP2, respectively, for 4 days. The graph shows qPCR of indicated gene expressions. For integrin inhibitor treatment, cells were treated with 10 uM of cRGDyK (Selleckchem, #S7844) for 10 minutes and washed with PBS before treated with RUCILP2. Data are presented as mean+/−SEM of one representative experiment done in technical triplicate. Statistical significance was determined by unpaired two-tailed Student's t test. *, p<0.05 vs 0 nM RUCILP2.

FIG. 10. Recombinant RUCILP2 reduces lipid content in adipocytes as indicated by oil red O staining. Staining was performed on 10% formalin-fixed adipocytes according to the manufacturer's protocol.

FIG. 11. Recombinant RUCILP2 stimulates sclerostin expression in MLO-Y4 (murine long bone osteocyte-Y4) cell line. The cells were incubated with FreeStyle293 medium for 4 hr and treated for 16 hr with indicated concentrations of RUCILP2 or irisin (Upper panel), with the addition at pretreatment with vehicle (phosphate-buffered saline) or 10 μM of integrin inhibitor cRGDyK for 10 min (Lower panel). Data are represented as mean±SEM, n=3 wells/group. Significant differences between two groups were evaluated using a two-tailed, unpaired Student's t test. Upper panel, *, p<0.05, **, p<0.01, #, p<0.05, ##, p<0.01, when compared with the blank group. Lower panel, *, p<0.05, when compared with the vehicle group.

FIG. 12. Recombinant RUCILP2 induces myotube formation in murine C2C12 myoblasts. The C2C12 myotubes were treated with indicated doses of RUCILP2 overnight and myotube images were collected at magnification, ×10.

FIG. 13. Recombinant RUCILP2 treatment reduces expression of genes involved in HepG2 liver cell gluconeogenesis, increases expression of genes involved in intestinal integration in Caco-2 cells, and stimulate gene expression in H9C2 cardiomyoblasts, respectively. The cells were treated with various doses of RUCILP2, and cells were collected for q-PCR quantification on indicated genes. For integrin inhibitor treatment, cells were treated with 10 uM of cRGDyK (Selleckchem, #S7844) for 10 minutes and washed with PBS before treated with RUCILP2. Data are presented as mean+/−SEM of one representative experiment done in technical triplicate. Statistical significance was determined by unpaired two-tailed Student's t-test. *, p<0.05 vs 0 nM RUCILP2.

FIG. 14. Recombinant RUCILP2 stimulates glucagon like peptide-1 (GLP-1) secretion in perfused rat colon when RUCILP2 is perfused through the luminal route. Left, GLP-1 secretion from the isolated perfused rat colon in the presence of RUCILP2, means±SEM, n=6 in each group. Right, baseline subtracted total GLP-1 output during 10-min (minutes 12-21) luminal infusions. **, p<0.01 using Student's t test.

FIG. 15. Recombinant RUCILP2 stimulates Peptide-YY (PYY) secretion in perfused rat colon when RUCILP2 is perfused through the luminal route. Left, PYY secretion from the isolated perfused rat colon in the presence of RUCILP2, means±SEM, n=6 in each group. Right, baseline subtracted total PYY output during 10-min (minutes 12-21) luminal infusions. **, p<0.01 using Student's t test.

FIG. 16. Recombinant RUCILP2 stimulates somatostatin secretion in perfused rat colon when RUCILP2 is perfused through the luminal route. Left, Somatostatin secretion from the isolated perfused rat colon in the presence of RUCILP2, means±SEM, n=6 in each group. Right, baseline subtracted total Somatostatin output during 10-min (minutes 12-21) luminal infusions. *, p<0.05 using Student's t test.

FIG. 17. Daily intraperitoneal injection for seven days of recombinant RUCILP2 into mice fed normal chow induces expression of genes involved in thermogenesis and reduces expression of a gene involved in lipogenesis. No effects on expression of marker genes for lipolysis. Recombinant RUCILP2 was daily injected intraperitoneal at a concentration of 1 mg/kg into 9-week-old wild type C57BL/6N mice for one week. The mRNA levels of indicated genes were analyzed by qRT-PCR. Data were expressed as mean±SEM, *, p<0.05; **, p<0.01; ***, p<0.001, when compared with phosphate-buffered saline (PBS) group, n=9 animals/group.

FIG. 18. Body weight change of mice treated with oral gavage of various strains of Ruminococcus torques species. R3-LD, R. torques ATCC 35915 at a dose of 5×10⁷ colony forming units per 100 μl; R3-HD, R. torques ATCC 35915 at a dose of 5×10⁸ colony forming units per 100 μl; R2-LD, R. torques ATCC 27756 at a dose of 5×10⁷ colony forming units per 100 μl; R2-HD, R. torques ATCC 27756 at a dose of 5×10⁸ colony forming units per 100 μl; HK-R2-HD, heat-killed R. torques ATCC 27756 at a dose of 5×10⁸colony forming units per 100 μl. Data are presented as mean+/−SEM. Thus, this figure shows that oral (gavage) administration of live or pasteurized Rumminoccocus torques strains have no significant effects on body weight development over a period of 8 weeks in mouse fed with chow diet (Altromin 1328 diet).

FIG. 19. Oral gavage of mice with Ruminococcus torques ATCC 27756 strain synthesizing RUMTOR_00181 reduces mouse body fat mass and increases lean body mass. The mice were fed normal chow and the interventions lasted for 8 weeks. Magnetic resonance imaging scanning of body composition in indicated groups of mice was performed according to the manufacturer's tutorial. R3-LD, R. torques ATCC 35915 at a dose of 5×10¹colony forming units per 100 μl; R3-HD, R. torques ATCC 35915 at a dose of 5×10⁸ colony forming units per 100 μl; R2-LD, R. torques ATCC 27756 at a dose of 5×10⁷ colony forming units per 100 μl; R2-HD, R. torques ATCC 27756 at a dose of 5×10⁸ colony forming units per 100 μl; HK-R2-HD, heat-killed R. torques ATCC 27756 at a dose of 5×10⁸colony forming units per 100 μl. Data are presented as mean+/−SEM. *, p<0.05, *, p<0.01, when compared with the PBS group determined by unpaired two-tailed Student's t test.

FIG. 20. Body weight development of high-fat diet-fed mice treated with oral gavage of various strains of Ruminococcus torques species. RT3, R. torques ATCC 35915 with absence of the gene encoding RUMTOR_00181 at a dose of 5×10⁹ colony forming units per 100 μl; Heat killed RT2, heat-killed R. torques ATCC 27756 with presence of the gene encoding RUMTOR_00181 at a dose of 5×10⁹ colony forming units per 100 μl; RT2, R. torques ATCC 27756 with the presence of the gene encoding RUMTOR_00181 at a dose of 5×10⁹ colony forming units per 100 μl. Data are presented as mean+/−SEM with 10 mice in each group. *, p<0.05 for RT2 vs RT3; #, p<0.05 for RT2 vs sterile phosphate-buffered saline; &, p<0.05 for RT2 vs heat-killed RT2. Statistical significance was determined by unpaired two-tailed t test. Thus, this figure shows that oral (gavage) supplementation of live Rumminoccocus torques strains significantly reduces body weight development over a period of 8 weeks in mouse fed with high-fat diet (Research Diet, D12451 i).

FIG. 21. RUMTOR_00181-producing Ruminococcus torques (RT ATCC 27756) strain improves glucose tolerance. The mice were fed normal chow. The glucose tolerance experiments were made at week 6. Left, intraperitoneal glucose tolerance test curve at week 6 after oral gavage of RUMTOR_00181-producing R. torques strain. Right, area under the curve for glucose tolerance test (GTT). R3-LD, R. torques ATCC 35915 at a dose of 5×10⁷ colony forming units per 100 μl; R3-HD, R. torques ATCC 35915 at a dose of 5×10⁸colony forming units per 100 μl; R2-LD, R. torques ATCC 27756 at a dose of 5×10⁷ colony forming units per 100 μl; R2-HD, R. torques ATCC 27756 at a dose of 5×10⁸colony forming units per 100 μl; HK-R2-HD, heat-killed R. torques ATCC 27756 at a dose of 5×10⁸ colony forming units per 100 μl. Data are presented as mean +/−SEM. *, indicates p<0.05 using Student's t test. i.p. means intraperitoneal injection. GTT means glucose tolerance test. AUC means area under the curve (here the curve of blood glucose excursions).

FIG. 22. RUMTOR_00181-producing Ruminococcus torques (RT ATCC 27756) strain improves glucose tolerance. The mice were fed with high-fat diet. The intraperitoneal glucose tolerance test was performed at week 6 after oral gavage of R. torques strains. RT3, R. torques ATCC 35915 with absence of the gene encoding RUMTOR_00181 at a dose of 5×10⁹ colony forming units per 100 μl; Heat-killed RT2, heat-killed R. torques ATCC 27756 with presence of the gene encoding RUMTOR_00181 at a dose of 5×10⁹ colony forming units per 100 μl; RT2, R. torques ATCC 27756 with presence of the gene encoding RUMTOR_00181 at a dose of 5×10⁹ colony forming units per 100 μl. Data are presented as mean+/−SEM with 10 mice in each group. *, p<0.05, ***, p<0.001 for RT2 vs RT3; #, p<0.05 for RT2 vs sterile phosphate-buffered saline; &, p<0.05 for RT2 vs heat-killed RT2. Statistical significance was determined by unpaired two-tailed t test. ip means intraperitoneal injection. GTT means glucose tolerance test.

FIG. 23. Oral gavage for eight weeks of a RUMTOR_00181-producing R. torques strain (RT ATCC 27756) to mice fed normal chow activates expression of genes involved in thermogenesis and reduces expression of genes of lipogenesis in inguinal fat cells. The q-PCR quantification was performed on RNA extracted from mouse inguinal adipose tissue. R3-LD, R. torques ATCC 35915 at a dose of 5×10⁷ colony forming units per 100 μl; R3-HD, R. torques ATCC 35915 at a dose of 5×10⁸ colony forming units per 100 μl; R2-LD, R. torques ATCC 27756 at a dose of 5×10⁷ colony forming units per 100 μl; R2-HD, R. torques ATCC 27756 at a dose of 5×10⁸ colony forming units per 100 μl; HK-R2-HD, heat-killed R. torques ATCC 27756 at a dose of 5×10⁸colony forming units per 100 μl. Data are presented as mean+/−SEM. n=3-4 mice per group. *, p<0.05 vs R. torques ATCC 35915 at 5×10⁸colony forming units per 100 μl group.

FIG. 24. Oral gavage for eight weeks of a RUMTOR_00181-producing R. torques strain (RT ATCC 27756) to mice fed normal chow causes an increase in cortical thickness of tibia bone. Left, 3D images of cross-section of mid-shaft tibia for each group. Right, cortical thickness collected from 3D images for each group. *, false discovery rate-corrected p<0.05. R3-LD, R. torques ATCC 35915 at a dose of 5×10′ colony forming units per 100 μl; R3-HD, R. torques ATCC 35915 at a dose of 5×10⁸ colony forming units per 100 μl; R2-LD, R. torques ATCC 27756 at a dose of 5×10⁷ colony forming units per 100 μl; R2-HD, R. torques ATCC 27756 at a dose of 5×10⁸ colony forming units per 100 μl; HK-R2-HD, heat-killed R. torques ATCC 27756 at a dose of 5×10⁸ colony forming units per 100 μl. Data are presented as mean+/−SEM. n=3-4 mice per group. *, p<0.05 determined by Student's t test.

FIG. 25. Representative chromatogram showing the presence of RUCILP2 in fasting human plasma. Parallel reaction monitoring (PRM) elution profile for y-ions for the unique tryptic RUCILP2 peptide (EAAGYNVYVDGVK) found in human plasma samples. Top panel is PRM trace for the fragmental ions (box a) of light peptide found in human plasma sample, bottom panel is PRM trace for the fragmental ions (box b) of 10.0 femtomole of heavy isotope labelled (Lys ¹³C6, ¹⁵N4) synthetic peptide (internal standard, IS) spiked into fractionated human plasma samples. Retention times for each peptide are labeled on the x axis, and y axis represents the relative intensity for each fragmental ion peak. One milliliter of human plasma sample was depleted of albumin and immunoglobulin G following deglycosylation. Deglycosylated plasma was resolved by SDS-PAGE and molecular mass regions corresponding to completely deglycosylated RUCILP2 (10-15 kDa) was excised and subsequently digested in-gel overnight before LC-MS/MS detection. Based on comparative analysis on relative intensities of fragmental ions found in processed human plasma and fragmental ions found in plasma spiked with heavy isotope labelled internal standards, we estimate the inter-individual concentrations of RUCILP2 in human plasma vary between 10 and 100 pg/ml.

FIG. 26. Amino acids sequence alignment between RUCILP2 and 21-AABP2. The multiple sequence alignments were performed by Clustal Omega. Sequence of 21-AABP2 is highlighted in grey.

FIG. 27. Molecular docking model of 21-AABP2 and integrin αV/β5 receptor. Dot lines indicate hydrogen bond formed by the two ligands and binding sites of 21-AABP2 were shown in amino acid residue codes. The best ZDOCK webserver (ZDOCK (https://zdock.umassmed.edu/) predicted model of RUCILP2 and integrin αV/β5 receptor complex was visualized in PyMOL (v2.1.1) program to show the binding amino acids residues at Y5, F6, E8, and N17, respectively, of 21-AABP2 to integrin αV/β5 receptor. This docking model predicted the potential binding as well the binding sites between 21-AABP2 and integrin αV/β5 receptor.

FIG. 28. The 21-AABP2 promotes expression of genes involved in thermogenesis/browning in human visceral white preadipocytes (HWP), induces expression of key genes regulating thermogenesis in mouse inguinal preadipocytes, and stimulates expression of genes involved in myogenesis of murine C2C12 myoblasts. White preadipocytes from human visceral fat (C-12732, PromoCell) were cultured until 80% confluent and switched into differentiation media (with 0.3 ml/ml of fetal calf serum (FCS), 8 ug/ml of d-biotin, 0.5ug/ml of insulin, 400 ng/ml of dexamethasone) in the presence of 15 nM of 21-AABP2. The differentiation process to mature adipocytes was completed after 14 days. Cells were harvested after 14 days of differentiation and the indicated genes were quantified by q-PCR. Inguinal fat tissues from 6-week-old wild-type C57BL/6J female mice were dissected and washed with PBS, minced and digested for 1 hour at 37° C. in PBS containing 10 mM CaCl₂, 2.4 U/ml dispase II (Roche) and 10 mg/ml collagenase D (Roche). After adding warm DMEM/F12 (1:1) with 10% FCS, digested tissue was filtered through a 70 mm cell strainer and centrifuged at 600×g for 10 minutes. Pellet was resuspended by 40 ml DMEM/F12 (1:1) with 10% FCS and filtered through a 40 mm cell strainer followed by centrifugation at 600×g for 10 minutes. Pelleted inguinal stromal vascular cells were grown to confluence and split onto 12-well plates. The cells were induced to differentiate by treatment with 1 mM rosiglitazone, 5 mM dexamethasone, 0.5 mM isobutyl methyl xanthine for 2 days. After that, cells were maintained in 1 mM rosiglitazone for 4 days with medium change every other day. The cells were treated with 15 nM of 21-AABP2 every other day during 6 days of differentiation. Cells were harvested after 6 days of differentiation and thermogenesis gene were quantified by q-PCR. C2C12 myoblasts (CRL-1772, ATCC) were cultured until 80% confluent and switched into differentiation media (with 2% horse serum). The treatment with 21-AABP2 started from the second day of differentiation. Cells were harvested after 4 days of differentiation and expression of myogenesis genes were quantified by q-PCR. Data are presented as mean+/−SEM of one representative experiment done in biological triplicates. Statistical significance was determined by unpaired two-tailed Student's t test, *, p<0.05

FIG. 29. 21-AABP2 increases myotube development in C2C12 murine myoblasts. Representative images of myoblasts (CRL-1772, ATCC) at 24h of differentiation in the presence of phosphate buffered saline (PBS, blank) or 21-AABP2 (15 nM). Presented images were from one representative experiment done in biological triplicates.

FIG. 30. Insulin release from the immortalized rat insulinoma INS-1 cells is increased following 21-AABP2 stimulation. INS-1 cells (832/13, ThermoFisher) were grown in in RPM 1640 medium until a confluency of 70% was achieved and switched to RPM 1640 medium supplied with 15 nM of 21-AABP2 and incubated for 12 hours. The insulin concentration in the supernatant of cell culture medium was measured by MSD rat/mouse insulin ELISA kit. Data are presented as mean+/−SEM of one representative experiment done in biological triplicate. Statistical significance was determined by unpaired two-tailed Student's t test, *, p<0.05.

FIG. 31. High-quality 3D structure of RUMTOR_00181 protein. SP, signal peptide, TD, transmembrane domain, FNIII, fibronectin-containing type III domain. Blue ribbons display non-annotated regions. The protein structure was modeled using the artificial intelligence algorithm AlphaFold2²² via ColabFold²³ and MMseqs2¹⁴ for predicting protein structure using multiple sequence alignments²³.

FIG. 32. Alignment of irisin to RUCILP1 and RUCILP2. (A) A total of 27 amino acids from RUCILP 1 (88 aa) are identical to that of irisin. (B) It is demonstrated 30 amino acid residues from RUCILP2 (87 aa) are identical to that of irisin. Identical residues between the two sequences are denoted by asterisks; low and high degrees of similarity are represented by a period and a colon, respectively.

FIG. 33. Alignment between RUCILP1 and RUCILP2 sequence. A total of 65 amino acids from RUCILP 1 (88 aa) is identical to that of RUCILP2 (87 aa). Identical residues between the two sequences are denoted by asterisks; low and high degrees of similarity are represented by a period and a colon, respectively.

FIG. 34. Proposed topology of RUMTOR_00181 protein and trypsin/LysC-dependent cleavage for the release of RUCILP1 and RUCILP2 to the bacterial extracellular space. aa, amino acid residues, K, lysine, LysC, endoproteinase that cleaves proteins on the C-terminal side of lysine residues.

FIG. 35. Oral gavage of mice with Ruminococcus torques ATCC 27756 strain synthesizing RUMTOR_00181 reduces body fat mass and increases lean body mass in mice fed a chow diet for eight weeks. The mice were fed normal chow and the interventions lasted for eight weeks. Magnetic Resonance Imaging (MRI) scanning of body composition in indicated groups of mice was performed according to the manufacturer's tutorial. PBS, phosphate-buffered saline; R3-LD, R. torques ATCC 35915 at a dose of 5×10⁷ colony forming units per 100 μl; R3-HD, R. torques ATCC 35915 at a dose of 5×10⁸ colony forming units per 100 μl; R2-LD, R. torques ATCC 27756 at a dose of 5×10⁷ colony forming units per 100 μl; R2-HD, R. torques ATCC 27756 at a dose of 5×10⁸ colony forming units per 100 μl; HK-R2-HD, heat-killed R. torques ATCC 27756 at a dose of 5×10⁸colony forming units per 100 μl. Data are presented as mean+/−SEM. *, p<0.05, and **, p<0.01 determined by unpaired two-tailed Student's t test.

FIG. 36. Oral gavage of high-fat diet-fed mice with ATCC 27756 Ruminoccocus torques strain synthesizing RUMTOR_00181 reduces tissue weight of inguinal and epididymal fat. PBS, phosphate-buffered saline; RT3, R. torques ATCC 35915 at a dose of 5×10⁹ colony forming units per 100 μl; Heat killed RT2, heat-killed R. torques ATCC 27756 at a dose of 5×10⁹ colony forming units per 100 μl; RT2, R. torques ATCC 27756 at a dose of 5×10¹colony forming units per 100 μl. Data are presented as mean+/−SEM with 10 mice in each group; iWAT, inguinal white adipose tissue; eWAT, epididymal white adipose tissue. Data are presented as mean+/−SEM. *, p<0.05, determined by one-way ANOVA followed by Turkey post hoc correction.

FIG. 37. Oral gavage of RUMTOR_00181-producing ATCC 27756 Ruminoccocus torques strain activates thermogenesis reduces lipogenesis, enhances lipolysis, and down-regulates inflammation in adipose tissue of mice fed with high-fat diet. PBS, phosphate-buffered saline; RT3, R. torques ATCC 35915 at a dose of 5×10⁹ colony forming units per 100 μl; Heat killed RT2, heat-killed R. torques ATCC 27756 at a dose of 5×10⁹ colony forming units per 100 μl; RT2, R. torques ATCC 27756 at a dose of 5×10⁹ colony forming units per 100 μl. Data are presented as mean+/−SEM with 10 mice in each group. ns, no significance, *, p<0.05; **, p<0.01; ***, p<0.001, determined using one-way ANOVA followed by Tukey post hoc correction.

FIG. 38. Oral gavage with RUMTOR_00181-producing ATCC 27756 Ruminoccocus torques strain reduces size of adipocytes in inguinal fat of mice fed with high-fat diet visualized by hematoxylin- and eosin-staining. PBS, phosphate-buffered saline; RT3, R. torques ATCC 35915 at a dose of 5×10⁹ colony forming units per 100 μl; Heat killed RT2, heat-killed R. torques ATCC 27756 at a dose of 5×10⁹ colony forming units per 100 μl; RT2, R. torques ATCC 27756 at a dose of 5×10⁹ colony forming units per 100 μl.

FIG. 39. Oral gavage of a RUMTOR_00181-producing ATCC 27756 Ruminoccocus torques strain enhances browning marker UCP1 expression at protein level in inguinal white adipose tissue of mice fed with high-fat diet. PBS, phosphate-buffered saline; RT3, R. torques ATCC 35915 at a dose of 5×10⁹ colony forming units per 100 μl; Heat killed RT2, heat-killed R. torques ATCC 27756 at a dose of 5×10⁹ colony forming units per 100 μl; RT2, R. torques ATCC 27756 at a dose of 5×10⁹ colony forming units per 100 μl. Data are presented as mean+/−SEM with 6 mice in each group. **, p<0.01; ***, p<0.001, determined using one-way ANOVA followed by Tukey post hoc correction.

FIG. 40. Oral gavage with a RUMTOR_00181-producing ATCC 27756 Ruminoccocus torques strain activates bone formation in distal femur of mice fed with high-fat diet. Upper panel demonstrates the representative 3D cross-sectional image of femur, lower panel summaries the comparisons of cortical thickness collected from 3D (left) and 2D (right) images. PBS, phosphate-buffered saline; RT3, R. torques ATCC 35915 at a dose of 5×10⁹ colony forming units per 100 μl; Heat killed RT2, heat-killed R. torques ATCC 27756 at a dose of 5×10⁹ colony forming units per 100 μl; RT2, R. torques ATCC 27756 at a dose of 5×10⁹ colony forming units per 100 μl. Data are presented as mean+/−SEM with 6 mice in each group. *, p<0.05; **, p<0.01; ***, p<0.001, determined using one-way ANOVA followed by Tukey post hoc correction.

FIG. 41. Images of SPOT peptide microarray (μSPOT) assay of binding of RUCILP1 and RUCILP2 to the integrin αV/β5 receptor. (A) Images of cellulose membrane without synthesized 15-mer peptides after direct incubation of 6x-His antibody. (B) Representative images of cellulose membranes attaching synthesized library of 15-mer peptides for RUCILP1 (left) and RUCILP2 (right), respectively, after interaction with integrin αV/β5 receptor, followed by incubation with 6x-His antibody.

FIG. 42. Outcome of systematic screening to identify the binding epitopes of RUCILPs to integrin αV/β5 receptor. (A) Relative integrin αV/β5 (2.5 nM) binding of 15-mer peptides of RUCILP1 (SEQ ID NO:s 22-95). (B) Integrin αV/β5 (2.5 nM) binding of 15-mer peptides of RUCILP2 (SEQ ID NO:s 96-168). Data are expressed as mean+SD of triplicated quantifications.

FIG. 43. AlphaFold 3D structures of RUCILPs. (A) Structure of RUCILP1 predicted by AlphaFold. (B) Structure of RUCILP2 predicted by AlphaFold. (C) Crystal structure of irisin. (D) Electrostatic surface representation of RUCILP1 (upper) and RUCILP2 (lower). In A-C, loops responsible for binding to integrin αV/β5 receptor are marked in red. In D, red region indicates flexible C terminus of both proteins.

FIG. 44. In vitro effects of RUCILP1 (panel A) and RUCILP2 (panel B) in gene expression studies of various cell types. The mRNA expression level of brown adipocyte-selective genes and white adipocyte marker gene on mouse 3T3-L1 fibroblasts, bone remodelling maker gene on mouse osteoblasts, and myotube formation genes on mouse myoblasts upon the treatment of RUCILP1 (A) and RUCILP2 (B). * indicates p<0.05 using one-way ANOVA followed by Dunnett post hoc correction. Data are expressed as mean±SEM, n=3 wells/group.

FIG. 45. Effects of 21-AABP1 on mouse 3T3-L1 fibroblasts. Data are expressed as mean±SEM, n=3 wells/group.

FIG. 46. Effects of RUCILP1 and RUCILP2 on gene expression in vivo. Recombinant RUCILPs were daily injected into peritoneum of 8-week-old wild type C57BL/6N mice at a concentration of 1 mg/kg for one week. The mRNA levels of indicated genes in subcutaneous white adipose tissue (SWAT) and liver were analyzed by qRT-PCR. n=6 animals per group. * indicates p<0.05 using one-way ANOVA followed by Dunnett post hoc correction. Data are expressed as mean±SEM.

FIG. 47. Alanine scanning of 19-mer epitopes in RUCILP1 and RUCILP2 to the integrin αV/β5 receptor. (A) Relative integrin αV/β5 (2.5 nM) binding to alanine scanning library of 19-mer epitopes of RUCILP1 (SEQ ID NO:s 169-188). (B) Relative integrin αV/β5 (2.5 nM) binding to alanine scanning library of 19-mer epitopes of RUCILP2 (SEQ ID NO:s 189-208). Data are expressed as mean±SEM of triplicated quantifications.

FIG. 48. Truncation scanning of 19-mer epitopes in RUCILP1 and RUCILP2 to the integrin αV/β5 receptor. (A) Relative integrin αV/β5 (2.5 nM) binding to truncation scanning library of 19-mer epitopes of RUCILP1 (SEQ ID NO: 209-241). (B) Relative integrin αV/β5 (2.5 nM) binding to truncation scanning library of 19-mer epitopes of RUCILP2 (SEQ ID NO: 242-274). Darkly coloured bars highlight enhanced binding hits after subtraction of background signals. Data are expressed as mean±SD of triplicated quantifications.

### DETAILED DESCRIPTION

### Definitions

Amino acid substitution—The term “amino acid substitution” as used herein refers to the change from one amino acid to a different amino acid in a peptide, polypeptide or protein. The substitution may be a conservative substitution, wherein an amino acid is exchanged into another amino acid that has similar properties. The substitution may also be a non-conservative substitution, wherein an amino acid is exchanged into another amino acid with different properties. Properties of an amino acid include for example the charge, polarity, acidity, size and hydrophobicity of the amino acid.

Bone disorders—The term “bone disorders” as used herein refers to a subgroup of musculoskeletal disorders, diseases, injuries and conditions that affect human bones. In particular, it refers to osteoporosis, osteogenesis imperfect and osteopetrosis. Osteoporosis can be divided into primary and secondary osteoporosis. Primary osteoporosis is the most common form of the disease and includes postmenopausal osteoporosis (type I), and senile osteoporosis (type II). There are numerous causes of secondary bone loss (osteoporosis), including adverse effects of various drug therapy, endocrine disorders, eating disorders, immobilization, bone marrow-related disorders, disorders of the gastrointestinal or biliary tract, renal disease, and cancer.

Identity—The term identity, with respect to a polynucleotide or polypeptide, are defined herein as the percentage of nucleic acids or amino acids in the candidate sequence that are identical, to the residues of a corresponding native nucleic acids or amino acids, after aligning the sequences and introducing gaps, if necessary, to achieve the maximum percent identity. Neither 5′ or 3′ extensions nor insertions (for nucleic acids) or N′ or C′ extensions nor insertions (for polypeptides) result in a reduction of identity. Methods and computer programs for the alignments are well known in the art.

Live Biopharmaceutical Product (LBP)—The term “Live Biopharmaceutical Product” or “LBP” as used herein refers to a biological product that:
i) contains live microorganisms, such as bacteria or yeasts;
ii) is applicable to the prevention, treatment, or cure of a disease or condition of human beings; and
iii) is not a vaccine, fecal microbiota transplant or gene therapy agent.

Metabolic disorders—The term “metabolic disorders” as used herein refers to a disorder that negatively alters the body's processing and distribution of macronutrients, such as proteins, fats and carbohydrates. In particular, the term ‘metabolic disorders’ as used herein refers to diseases, disorders and conditions related to metabolic syndrome.

Metabolic syndrome—The term “metabolic syndrome” as used herein refers to a complication of a series of pathological conditions, including obesity, high blood pressure, high blood sugar, high serum triglycerides and low serum high-density lipoprotein, as well as cardiovascular disease, FLD, prediabetes and T2D. Related concepts such as syndrome X, insulin resistance syndrome, visceral fat syndrome, and multiple risk factor syndrome are also included in the “metabolic syndrome” as used in the present invention. In the present invention, prevention or treatment of metabolic syndrome means prevention or treatment of occurrence of the symptoms in at least one pathological conditions selected from the group of the pathological conditions as mentioned above.

Muscle disorders—The term “muscle disorders” as used herein refers to a subgroup of musculoskeletal disorders, diseases, injuries and conditions, as well as neuromuscular disorders, which affect human joints and muscles. In particular, it refers to muscular dystrophy, such as Duchenne muscular dystrophy, amyotrophic lateral sclerosis (ALS), Lambert-Eaton syndrome (Lambert-Eaton myasthenic syndrome), myasthenia gravis, polymyositis, and peripheral neuropathy.

Prediabetes—The term “prediabetes” as used herein refers to a condition characterized by elevated blood sugar levels. Many, but not all, of the patients suffering from prediabetes will develop T2D. Prediabetes can be diagnosed by measuring haemoglobin A1C, fasting glucose, or glucose tolerance test, wherein results indicating prediabetes are an A1C of 5.7-6.4%, fasting blood sugar of 100-125 mg/dl, and oral glucose tolerance test (OGTT) 2 hour blood sugar of 140-199 mg/dl.

Treatment—The term “treatment” as used herein may refer to any kind of treatment. The treatment may be a curative treatment; it may also be an ameliorating treatment and/or a treatment reducing the effects of the treated disease, injury and/or disorder. The treatment may also be a treatment that delays progression and/or development of the treated disease, injury and/or disorder. The treatment may also be preventative/prophylactic, i.e. a treatment to eliminate or reduce the risk of developing the diseases, injuries and/or disorders disclosed herein.

### Polypeptide

The present invention relates to a polypeptide derived from the fibronectin type III domain-containing protein 5 (FNDC5) or RUMTOR_00181 (UniProt ID: A5KIY5) of the gut bacterial strain Ruminococcus torques. In particular, the present invention relates to a polypeptide comprising a fragment of the FNDC5 polypeptide or RUMTOR_00181 (RUCILP1; RUCILP2; Ruminococcus torques Irisin-Like Peptide 1 or 2), as well as variants and fragments thereof, such as for example a 21 amino acid fragment of RUCILP1 (21-AABP1; 21-amino acid bacterial peptide 1) or RUCILP2 (21-AABP2; 21-amino acid bacterial peptide 2), as well as fragments and variants of 21-AABP2 or 21AABP1.

Thus, provided herein is an isolated polypeptide having a length of less than 200 amino acids comprising or consisting of an amino acid sequence selected from the group consisting of:
a. the amino acid sequence according to SEQ ID NO: 4 and/or SEQ ID NO: 19;
b. a variant of SEQ ID NO: 4 and/or SEQ ID NO: 19, wherein said variant has at least 60%, such as at least 70%, such as at least 75%, such as at least 80%, such as at least 85%, such as at least 90%, such as at least 95% sequence identity to SEQ ID NO: 4 and/or SEQ ID NO: 19, but less than 99% sequence identity to SEQ ID NO: 4 and/or SEQ ID NO: 19;
c. a variant of SEQ ID NO: 4 and/or SEQ ID NO: 19, wherein said variant has between 1 and 40 amino acid substitutions relative to SEQ ID NO: 4 and/or SEQ ID NO: 19, such as 5, 10, 15, 20, 25, 30, or 35 amino acid substitutions relative to SEQ ID NO: 4 and/or SEQ ID NO: 19;
d. a fragment of SEQ ID NO: 4 and/or SEQ ID NO: 19 having a length of at least 10 amino acids, or a variant of said fragment having between 1 and 5 amino acid substitutions relative to SEQ ID NO: 4 and/or SEQ ID NO: 19, respectively, such as 1, 2 or 3 amino acid substitutions relative to SEQ ID NO: 4 and/or SEQ ID NO: 19, wherein said polypeptide has a length of less than 50 amino acids;
e. an amino acid sequence differing from SEQ ID NO: 4 and/or SEQ ID NO: 19 by truncation at the N-terminus by at least one amino acid, such as between 1-67 amino acids, such as between 1-60 amino acids, for example between 1-50 amino acids, such as between 1-40 amino acids, for example between 1-30 amino acids, such as between 1-20 amino acids, for example between 1-10 amino acids, such as between 1-5 amino acids, or a variant thereof having between 1 and 10 amino acid substitutions relative to SEQ ID NO: 4 and/or SEQ ID NO: 19, such as 1, 2, 3, 4, 5, 6, 7, 8 or 9 amino acid substitutions relative to SEQ ID NO: 4 and/or SEQ ID NO: 19;
f. an amino acid sequence differing from SEQ ID NO: 4 and/or SEQ ID NO: 19 by truncation at the C-terminus by at least one amino acid, such as between 1-21 amino acids, such as between 1-20 amino acids, for example between 1-15 amino acids, such as between 1-10 amino acids, for example between 1-5 amino acids, or a variant thereof having between 1 and 30 amino acid substitutions relative to SEQ ID NO: 4 and/or SEQ ID NO: 19, such as 1, 5, 10, 15, 20 or 25 amino acid substitutions relative to SEQ ID NO: 4 and/or SEQ ID NO: 19;
g. an amino acid sequence differing from SEQ ID NO: 4 and/or SEQ ID NO: 19 by truncation at the N-terminus by at least one amino acid, 1-67 amino acids, such as between 1-60 amino acids, for example between 1-50 amino acids, such as between 1-40 amino acids, for example between 1-30 amino acids, such as between 1-20 amino acids, for example between 1-10 amino acids, such as between 1-5 amino acids, and at the C-terminus by at least one amino acid, such as between 1-21 amino acids, such as between 1-20 amino acids, for example between 1-15 amino acids, such as between 1-10 amino acids, for example between 1-5 amino acids, wherein said polypeptide has a length of at least 10 amino acids, or a variant thereof having between 1 and 5 amino acid substitutions relative to SEQ ID NO: 4 and/or SEQ ID NO: 19, such as 1, 2 or 3 amino acid substitutions relative to SEQ ID NO: 4 and/or SEQ ID NO: 19;
h. the amino acid sequence according to SEQ ID NO: 5 and/or SEQ ID NO: 20;
i. a variant of SEQ ID NO: 5 and/or SEQ ID NO: 20, wherein said variant has at least 70%, such as at least 75%, such as at least 80%, such as at least 85%, such as at least 90%, such as at least 95% sequence identity to SEQ ID NO: 5 and/or SEQ ID NO: 20, but less than 99% sequence identity to SEQ ID NO: 5 and/or SEQ ID NO: 20;
j. a variant of SEQ ID NO: 5 and/or SEQ ID NO: 20, wherein said variant has between 1 and 10 amino acid substitutions relative to SEQ ID NO: 5 and/or SEQ ID NO: 20, such as 1, 2, 3, 4, 5, 6, 7, 8, 9 or 10 amino acid substitutions relative to SEQ ID NO: 5 and/or SEQ ID NO: 20, wherein said polypeptide has a length of less than 50 amino acids;
k. a fragment of SEQ ID NO: 5 and/or SEQ ID NO: 20 comprising at least 10 consecutive amino acids of SEQ ID NO: 5 and/or SEQ ID NO: 20, or a variant thereof having between 1 and 5 amino acid substitutions relative to SEQ ID NO: 5 and/or SEQ ID NO: 20, such as 1, 2, 3, or 4 amino acid substitutions relative to SEQ ID NO: 5 and/or SEQ ID NO: 20, wherein said polypeptide has a length of less than 50 amino acids;
l. a fragment of SEQ ID NO: 19, wherein said fragment is selected from the group consisting of SEQ ID NOs: 27, 33, 34, 35, 36, 37 and 95, and respective variants thereof having between 1 and 3 amino acid substitutions relative to SEQ ID NO: 19, such as 1, 2 or 3 amino acid substitutions relative to SEQ ID NO: 19, wherein said polypeptide has a length of less than 50 amino acids;
m. a fragment of SEQ ID NO: 4, wherein said fragment is selected from the group consisting of SEQ ID NOs: 107, 108, 109, 110, 111, 165 and 168, and respective variants thereof having between 1 and 3 amino acid substitutions relative to SEQ ID NO: 4, such as 1, 2 or 3 amino acid substitutions relative to SEQ ID NO: 4, wherein said polypeptide has a length of less than 50 amino acids;
n. a fragment of a variant of SEQ ID NO: 19, wherein said fragment is selected from the group consisting of SEQ ID NOs: 173, 176, 181 and 188, and respective variants thereof having between 1 and 3 amino acid substitutions relative to SEQ ID NO: 19, such as 1, 2 or 3 amino acid substitutions relative to SEQ ID NO: 19, wherein said polypeptide has a length of less than 50 amino acids;
o. a fragment of a variant of SEQ ID NO: 4, wherein said fragment is selected from the group consisting of SEQ ID NOs: 193, 196, 201 and 208, and respective variants thereof having between 1 and 3 amino acid substitutions relative to SEQ ID NO: 4, such as 1, 2 or 3 amino acid substitutions relative to SEQ ID NO: 4, wherein said polypeptide has a length of less than 50 amino acids;
p. a fragment of SEQ ID NO: 19, wherein said fragment is selected from the group consisting of SEQ ID NOs: 210, 211, 212, 213, 229, 232, 233, 234 and 235, and respective variants thereof having between 1 and 3 amino acid substitutions relative to SEQ ID NO: 19, such as 1, 2 or 3 amino acid substitutions relative to SEQ ID NO: 19, wherein said polypeptide has a length of less than 50 amino acids; and
q. a fragment of SEQ ID NO: 4, wherein said fragment is selected from the group consisting of SEQ ID NOs: 243, 244, 245, 246, 262, 265, 266, 267 and 268, and respective variants thereof having between 1 and 3 amino acid substitutions relative to SEQ ID NO: 4, such as 1, 2 or 3 amino acid substitutions relative to SEQ ID NO: 4, wherein said polypeptide has a length of less than 50 amino acids.

In one embodiment, the polypeptide has a length of at least 10 amino acids, such as at least 15, 20, 25, 30, 35, 40, 45, 50, 55, 60, 65, 70, 75, 80, 85, 90, 95 or at least 100 amino acids.

Thus, in one embodiment, the polypeptide has a length of at least 15 amino acid.

In one embodiment, the polypeptide has a length of at least 20 amino acids.

In one embodiment, the polypeptide has a length of at least 25 amino acids.

In one embodiment, the polypeptide has a length of at least 30 amino acids.

In one embodiment, the polypeptide has a length of at least 35 amino acids.

In one embodiment, the polypeptide has a length of at least 40 amino acids.

In one embodiment, the polypeptide has a length of at least 45 amino acids.

In one embodiment, the polypeptide has a length of at least 50 amino acids.

In one embodiment, the polypeptide has a length of at least 55 amino acids.

In one embodiment, the polypeptide has a length of at least 60 amino acids.

In one embodiment, the polypeptide has a length of at least 65 amino acids.

In one embodiment, the polypeptide has a length of at least 70 amino acids.

In one embodiment, the polypeptide has a length of at least 75 amino acids.

In one embodiment, the polypeptide has a length of at least 80 amino acids.

In one embodiment, the polypeptide has a length of at least 85 amino acids.

In one embodiment, the polypeptide has a length of at least 90 amino acids.

In one embodiment, the polypeptide has a length of at least 95 amino acids.

In one embodiment, the polypeptide has a length of at least 100 amino acids.

In one embodiment, the polypeptide has a length of less than 150 amino acids, such as less than 140,130,120,110,100, 95, 90, 85, 80, 75, 70, 65, 60, 55, 50, 45, 40, 35, 30, or less than 25, 20, 15 or 10 amino acid. Thus, in one embodiment, the polypeptide has a length of less than 150 amino acids.

In one embodiment, the polypeptide has a length of less than 140 amino acids.

In one embodiment, the polypeptide has a length of less than 130 amino acids.

In one embodiment, the polypeptide has a length of less than 120 amino acids.

In one embodiment, the polypeptide has a length of less than 110 amino acids.

In one embodiment, the polypeptide has a length of less than 100 amino acids.

In one embodiment, the polypeptide has a length of less than 95 amino acids.

In one embodiment, the polypeptide has a length of less than 90 amino acids.

In one embodiment, the polypeptide has a length of less than 85 amino acids.

In one embodiment, the polypeptide has a length of less than 80 amino acids.

In one embodiment, the polypeptide has a length of less than 75 amino acids.

In one embodiment, the polypeptide has a length of less than 70 amino acids.

In one embodiment, the polypeptide has a length of less than 65 amino acids.

In one embodiment, the polypeptide has a length of less than 60 amino acids.

In one embodiment, the polypeptide has a length of less than 55 amino acids.

In one embodiment, the polypeptide has a length of less than 50 amino acids.

In one embodiment, the polypeptide has a length of less than 45 amino acids.

In one embodiment, the polypeptide has a length of less than 40 amino acids.

In one embodiment, the polypeptide has a length of less than 35 amino acids.

In one embodiment, the polypeptide has a length of less than 30 amino acids.

In one embodiment, the polypeptide has a length of less than 25 amino acids.

In one embodiment, the polypeptide has a length of less than 20 amino acids.

In one embodiment, the polypeptide has a length of less than 15 amino acids.

In one embodiment, the polypeptide has a length of less than 10 amino acids.

In one embodiment, the polypeptide has a length between 10-200 amino acids, such as 10-150, such as 10-100, such as 10-80, such as 10-50, such as 10-30, such as 10-15, such as 25-75, such as 25-60, such as 30-80, such as 40-70, such as 15-30, such as 15-25, such as 18-23, such as 20-22, such as 50-150, such as 50-100, such as 70-100, such as 80-90, such as 85-90, such as 86-88 amino acids.

Thus, in one embodiment, the polypeptide has a length between 10-200 amino acids.

In one embodiment, the polypeptide has a length between 10-150 amino acids.

In one embodiment, the polypeptide has a length between 10-100 amino acids.

In one embodiment, the polypeptide has a length between 10-80 amino acids.

In one embodiment, the polypeptide has a length between 10-50 amino acids.

In one embodiment, the polypeptide has a length between 10-30 amino acids.

In one embodiment, the polypeptide has a length between 10-15 amino acids.

In one embodiment, the polypeptide has a length between 25-75 amino acids.

In one embodiment, the polypeptide has a length between 25-60 amino acids.

In one embodiment, the polypeptide has a length between 30-80 amino acids.

In one embodiment, the polypeptide has a length between 40-70 amino acids.

In one embodiment, the polypeptide has a length between 15-30 amino acids.

In one embodiment, the polypeptide has a length between 15-25 amino acids.

In one embodiment, the polypeptide has a length between 18-23 amino acids.

In one embodiment, the polypeptide has a length between 20-22 amino acids.

In one embodiment, the polypeptide has a length between 50-150 amino acids.

In one embodiment, the polypeptide has a length between 50-100 amino acids.

In one embodiment, the polypeptide has a length between 70-100 amino acids.

In one embodiment, the polypeptide has a length between 80-90 amino acids.

In one embodiment, the polypeptide has a length between 85-95 amino acids.

In one embodiment, the polypeptide has a length between 86-88 amino acids.

In one embodiment, the variant of the polypeptide has at least 60% sequence identity to SEQ ID NO: 4 or SEQ ID NO: 19, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% sequence identity to SEQ ID NO: 4 or SEQ ID NO: 19.

Thus, in one embodiment, the variant of the polypeptide has at least 60% sequence identity to SEQ ID NO: 4 or SEQ ID NO: 19. In one embodiment, the variant of the polypeptide has at least 61% sequence identity to SEQ ID NO: 4 or SEQ ID NO: 19. In one embodiment, the variant of the polypeptide has at least 62% sequence identity to SEQ ID NO: 4 or SEQ ID NO: 19. In one embodiment, the variant of the polypeptide has at least 63% sequence identity to SEQ ID NO: 4 or SEQ ID NO: 19. In one embodiment, the variant of the polypeptide has at least 64% sequence identity to SEQ ID NO: 4 or SEQ ID NO: 19. In one embodiment, the variant of the polypeptide has at least 65% sequence identity to SEQ ID NO: 4 or SEQ ID NO: 19. In one embodiment, the variant of the polypeptide has at least 66% sequence identity to SEQ ID NO: 4 or SEQ ID NO: 19. In one embodiment, the variant of the polypeptide has at least 67% sequence identity to SEQ ID NO: 4 or SEQ ID NO: 19. In one embodiment, the variant of the polypeptide has at least 68% sequence identity to SEQ ID NO: 4 or SEQ ID NO: 19. In one embodiment, the variant of the polypeptide has at least 69% sequence identity to SEQ ID NO: 4 or SEQ ID NO: 19. In one embodiment, the variant of the polypeptide has at least 70% sequence identity to SEQ ID NO: 4 or SEQ ID NO: 19. In one embodiment, the variant of the polypeptide has at least 71% sequence identity to SEQ ID NO: 4 or SEQ ID NO: 19. In one embodiment, the variant of the polypeptide has at least 72% sequence identity to SEQ ID NO: 4 or SEQ ID NO: 19. In one embodiment, the variant of the polypeptide has at least 73% sequence identity to SEQ ID NO: 4 or SEQ ID NO: 19. In one embodiment, the variant of the polypeptide has at least 74% sequence identity to SEQ ID NO: 4 or SEQ ID NO: 19. In one embodiment, the variant of the polypeptide has at least 75% sequence identity to SEQ ID NO: 4 or SEQ ID NO: 19. In one embodiment, the variant of the polypeptide has at least 76% sequence identity to SEQ ID NO: 4 or SEQ ID NO: 19. In one embodiment, the variant of the polypeptide has at least 77% sequence identity to SEQ ID NO: 4 or SEQ ID NO: 19. In one embodiment, the variant of the polypeptide has at least 78% sequence identity to SEQ ID NO: 4 or SEQ ID NO: 19. In one embodiment, the variant of the polypeptide has at least 79% sequence identity to SEQ ID NO: 4 or SEQ ID NO: 19. In one embodiment, the variant of the polypeptide has at least 80% sequence identity to SEQ ID NO: 4 or SEQ ID NO: 19. In one embodiment, the variant of the polypeptide has at least 81% sequence identity to SEQ ID NO: 4 or SEQ ID NO: 19. In one embodiment, the variant of the polypeptide has at least 82% sequence identity to SEQ ID NO: 4 or SEQ ID NO: 19. In one embodiment, the variant of the polypeptide has at least 83% sequence identity to SEQ ID NO: 4 or SEQ ID NO: 19. In one embodiment, the variant of the polypeptide has at least 84% sequence identity to SEQ ID NO: 4 or SEQ ID NO: 19. In one embodiment, the variant of the polypeptide has at least 85% sequence identity to SEQ ID NO: 4 or SEQ ID NO: 19. In one embodiment, the variant of the polypeptide has at least 86% sequence identity to SEQ ID NO: 4 or SEQ ID NO: 19. In one embodiment, the variant of the polypeptide has at least 87% sequence identity to SEQ ID NO: 4 or SEQ ID NO: 19. In one embodiment, the variant of the polypeptide has at least 88% sequence identity to SEQ ID NO: 4 or SEQ ID NO: 19. In one embodiment, the variant of the polypeptide has at least 89% sequence identity to SEQ ID NO: 4 or SEQ ID NO: 19. In one embodiment, the variant of the polypeptide has at least 90% sequence identity to SEQ ID NO: 4 or SEQ ID NO: 19. In one embodiment, the variant of the polypeptide has at least 91% sequence identity to SEQ ID NO: 4 or SEQ ID NO: 19. In one embodiment, the variant of the polypeptide has at least 92% sequence identity to SEQ ID NO: 4 or SEQ ID NO: 19. In one embodiment, the variant of the polypeptide has at least 93% sequence identity to SEQ ID NO: 4 or SEQ ID NO: 19. In one embodiment, the variant of the polypeptide has at least 94% sequence identity to SEQ ID NO: 4 or SEQ ID NO: 19. In one embodiment, the variant of the polypeptide has at least 95% sequence identity to SEQ ID NO: 4 or SEQ ID NO: 19. In one embodiment, the variant of the polypeptide has at least 96% sequence identity to SEQ ID NO: 4 or SEQ ID NO: 19. In one embodiment, the variant of the polypeptide has at least 97% sequence identity to SEQ ID NO: 4 or SEQ ID NO: 19. In one embodiment, the variant of the polypeptide has at least 98% sequence identity to SEQ ID NO: 4 or SEQ ID NO: 19. In one embodiment, the variant of the polypeptide has at least 99% sequence identity to SEQ ID NO: 4 or SEQ ID NO: 19.

In one embodiment, the variant of the polypeptide has less than 99% sequence identity to SEQ ID NO: 4 or SEQ ID NO: 19.

In one embodiment, the variant of the polypeptide has between 1 and 25 amino acid substitutions as compared to SEQ ID NO: 4 or SEQ ID NO: 19, such as between 1 and 20, such as between 1 and 15, such as between 1 and 10, such as between 1 and 5, such as between 1 and 3, such as between 10 and 20, such as between 5 and 15, such as between 5 and 10 amino acid substitutions as compared to SEQ ID NO: 4 or SEQ ID NO: 19.

Thus, in one embodiment, the variant of the polypeptide has between 1 and 25 amino acid substitutions as compared to SEQ ID NO: 4 or SEQ ID NO: 19.

In one embodiment, the variant of the polypeptide has between 1 and 20 amino acid substitutions as compared to SEQ ID NO: 4 or SEQ ID NO: 19.

In one embodiment, the variant of the polypeptide has between 1 and 15 amino acid substitutions as compared to SEQ ID NO: 4 or SEQ ID NO: 19.

In one embodiment, the variant of the polypeptide has between 1 and 10 amino acid substitutions as compared to SEQ ID NO: 4 or SEQ ID NO: 19.

In one embodiment, the variant of the polypeptide has between 1 and 5 amino acid substitutions as compared to SEQ ID NO: 4 or SEQ ID NO: 19.

In one embodiment, the variant of the polypeptide has between 1 and 3 amino acid substitutions as compared to SEQ ID NO: 4 or SEQ ID NO: 19.

In one embodiment, the variant of the polypeptide has between 10 and 20 amino acid substitutions as compared to SEQ ID NO: 4 or SEQ ID NO: 19.

In one embodiment, the variant of the polypeptide has between 5 and 15 amino acid substitutions as compared to SEQ ID NO: 4 or SEQ ID NO: 19.

In one embodiment, the variant of the polypeptide has between 5 and 10 amino acid substitutions as compared to SEQ ID NO: 4 or SEQ ID NO: 19.

In some embodiments, the isolated polypeptide comprises both the sequences of SEQ ID NO: 4 and SEQ ID NO: 19, or the respective variants or fragments thereof as described herein.

In one embodiment, the variant of the polypeptide has at least 90% sequence identity to SEQ ID NO: 5 or SEQ ID NO: 20, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% sequence identity to SEQ ID NO: 5 or SEQ ID NO: 20.

Thus, in one embodiment, the variant of the polypeptide has at least 60% sequence identity to SEQ ID NO: 5 or SEQ ID NO: 20. In one embodiment, the variant of the polypeptide has at least 61% sequence identity to SEQ ID NO: 5 or SEQ ID NO: 20. In one embodiment, the variant of the polypeptide has at least 62% sequence identity to SEQ ID NO: 5 or SEQ ID NO: 20. In one embodiment, the variant of the polypeptide has at least 63% sequence identity to SEQ ID NO: 5 or SEQ ID NO: 20. In one embodiment, the variant of the polypeptide has at least 65% sequence identity to SEQ ID NO: 5 or SEQ ID NO: 20. In one embodiment, the variant of the polypeptide has at least 65% sequence identity to SEQ ID NO: 5 or SEQ ID NO: 20. In one embodiment, the variant of the polypeptide has at least 66% sequence identity to SEQ ID NO: 5 or SEQ ID NO: 20. In one embodiment, the variant of the polypeptide has at least 67% sequence identity to SEQ ID NO: 5 or SEQ ID NO: 20. In one embodiment, the variant of the polypeptide has at least 68% sequence identity to SEQ ID NO: 5 or SEQ ID NO: 20. In one embodiment, the variant of the polypeptide has at least 69% sequence identity to SEQ ID NO: 5 or SEQ ID NO: 20. In one embodiment, the variant of the polypeptide has at least 70% sequence identity to SEQ ID NO: 5 or SEQ ID NO: 20. In one embodiment, the variant of the polypeptide has at least 71% sequence identity to SEQ ID NO: 5 or SEQ ID NO: 20. In one embodiment, the variant of the polypeptide has at least 72% sequence identity to SEQ ID NO: 5 or SEQ ID NO: 20. In one embodiment, the variant of the polypeptide has at least 73% sequence identity to SEQ ID NO: 5 or SEQ ID NO: 20. In one embodiment, the variant of the polypeptide has at least 75% sequence identity to SEQ ID NO: 5 or SEQ ID NO: 20. In one embodiment, the variant of the polypeptide has at least 75% sequence identity to SEQ ID NO: 5 or SEQ ID NO: 20. In one embodiment, the variant of the polypeptide has at least 76% sequence identity to SEQ ID NO: 5 or SEQ ID NO: 20. In one embodiment, the variant of the polypeptide has at least 77% sequence identity to SEQ ID NO: 5 or SEQ ID NO: 20. In one embodiment, the variant of the polypeptide has at least 78% sequence identity to SEQ ID NO: 5 or SEQ ID NO: 20. In one embodiment, the variant of the polypeptide has at least 79% sequence identity to SEQ ID NO: 5 or SEQ ID NO: 20. In one embodiment, the variant of the polypeptide has at least 80% sequence identity to SEQ ID NO: 5 or SEQ ID NO: 20. In one embodiment, the variant of the polypeptide has at least 81% sequence identity to SEQ ID NO: 5 or SEQ ID NO: 20. In one embodiment, the variant of the polypeptide has at least 82% sequence identity to SEQ ID NO: 5 or SEQ ID NO: 20. In one embodiment, the variant of the polypeptide has at least 83% sequence identity to SEQ ID NO: 5 or SEQ ID NO: 20. In one embodiment, the variant of the polypeptide has at least 85% sequence identity to SEQ ID NO: 5 or SEQ ID NO: 20. In one embodiment, the variant of the polypeptide has at least 85% sequence identity to SEQ ID NO: 5 or SEQ ID NO: 20. In one embodiment, the variant of the polypeptide has at least 86% sequence identity to SEQ ID NO: 5 or SEQ ID NO: 20. In one embodiment, the variant of the polypeptide has at least 87% sequence identity to SEQ ID NO: 5 or SEQ ID NO: 20. In one embodiment, the variant of the polypeptide has at least 88% sequence identity to SEQ ID NO: 5 or SEQ ID NO: 20. In one embodiment, the variant of the polypeptide has at least 89% sequence identity to SEQ ID NO: 5 or SEQ ID NO: 20. In one embodiment, the variant of the polypeptide has at least 90% sequence identity to SEQ ID NO: 5 or SEQ ID NO: 20. In one embodiment, the variant of the polypeptide has at least 91% sequence identity to SEQ ID NO: 5 or SEQ ID NO: 20. In one embodiment, the variant of the polypeptide has at least 92% sequence identity to SEQ ID NO: 5 or SEQ ID NO: 20. In one embodiment, the variant of the polypeptide has at least 93% sequence identity to SEQ ID NO: 5 or SEQ ID NO: 20. In one embodiment, the variant of the polypeptide has at least 95% sequence identity to SEQ ID NO: 5 or SEQ ID NO: 20. In one embodiment, the variant of the polypeptide has at least 95% sequence identity to SEQ ID NO: 5 or SEQ ID NO: 20. In one embodiment, the variant of the polypeptide has at least 96% sequence identity to SEQ ID NO: 5 or SEQ ID NO: 20. In one embodiment, the variant of the polypeptide has at least 97% sequence identity to SEQ ID NO: 5 or SEQ ID NO: 20. In one embodiment, the variant of the polypeptide has at least 98% sequence identity to SEQ ID NO: 5 or SEQ ID NO: 20. In one embodiment, the variant of the polypeptide has at least 99% sequence identity to SEQ ID NO: 5 or SEQ ID NO: 20.

In one embodiment, the variant of the polypeptide has less than 99% sequence identity to SEQ ID NO: 5 or SEQ ID NO: 20.

In one embodiment, the variant of the polypeptide has between 1 and 5 amino acid substitutions as compared to SEQ ID NO: 5 or SEQ ID NO: 20, such as between 1 and 4, such as between 1 and 3, such as between 2 and 4, such as between 2 and 5, such as between 3 and 5 amino acid substitutions as compared to SEQ ID NO: 5 or SEQ ID NO: 20.

Thus, in one embodiment, the variant of the polypeptide has between 1 and 5 amino acid substitutions as compared to SEQ ID NO: 5 or SEQ ID NO: 20.

In one embodiment, the variant of the polypeptide has between 1 and 4 amino acid substitutions as compared to SEQ ID NO: 5 or SEQ ID NO: 20.

In one embodiment, the variant of the polypeptide has between 1 and 3 amino acid substitutions as compared to SEQ ID NO: 5 or SEQ ID NO: 20.

In one embodiment, the variant of the polypeptide has between 2 and 4 amino acid substitutions as compared to SEQ ID NO: 5 or SEQ ID NO: 20.

In one embodiment, the variant of the polypeptide has between 2 and 5 amino acid substitutions as compared to SEQ ID NO: 5 or SEQ ID NO: 20.

In one embodiment, the variant of the polypeptide has between 3 and 5 amino acid substitutions as compared to SEQ ID NO: 5 or SEQ ID NO: 20.

In some embodiments, the isolated polypeptide comprises both the sequences of SEQ ID NO: 5 and SEQ ID NO: 20, or the respective variants or fragments thereof as described herein.

In one embodiment, the fragment of the polypeptide comprises or consists of an amino acid sequence according to positions 7 to 16 of SEQ ID NO: 4, corresponding to SEQ ID NO: 6, or a variant thereof having between 1 and 5 amino acid substitutions as compared to SEQ ID NO: 4, such as 1, 2, 3, 4 or 5 amino acid substitutions as compared to SEQ ID NO: 4.

In one embodiment, the fragment of the polypeptide comprises or consists of an amino acid sequence according to positions 27 to 39 of SEQ ID NO: 4, corresponding to SEQ ID NO: 7, or a variant thereof having between 1 and 6 amino acid substitutions as compared to SEQ ID NO: 4, such as 1, 2, 3, 4, 5 or 6 amino acid substitutions as compared to SEQ ID NO: 4.

In one embodiment, the fragment of the polypeptide comprises or consists of an amino acid sequence according to positions 43 to 56 of SEQ ID NO: 4, corresponding to SEQ ID NO: 8, or a variant thereof having between 1 and 6 amino acid substitutions as compared to SEQ ID NO: 4, such as 1, 2, 3, 4, 5 or 6 amino acid substitutions as compared to SEQ ID NO: 4.

In one embodiment, the amino acid substitutions are conservative substitutions. A conservative amino acid substitution is a replacement of an amino acid in a polypeptide to a given amino acid with similar biochemical properties, such as for example similar size, charge, hydrophobicity and/or polarity. Such substitutions often have a smaller effect on polypeptide function compared to non-conservative substitutions. Examples of conservative amino acid substitutions can be seen in the table below.

### Examples of Conservative Amino Acid Substitutions.

| Amino acid | Conservative substitution |
|---|---|
| A | G, S, T, V |
| C | S, T, M |
| D | S, K, Q, H, N, E |
| E | P, D, S, R, K, Q, H, N |
| F | M, V, I, L, W, Y |
| G | A, S, N |
| H | D, E, N, M, R, Q |
| I | M, V, Y, F, L |
| K | D, E, N, Q, R |
| L | M, V, I, Y, F |
| M | H, Q, Y, F, L, I, V |
| N | G, D, E, T, S, R, K, Q, H |
| P | E |
| Q | D, E, N, H, M, S, R, K |
| R | E, N, H, Q, K |
| S | G, D, E, N, Q, A, T |
| T | N, S, V, A |
| Y | H, M, I, L, F, W |
| V | T, A, M, F, L, I |
| W | F, Y |

In other embodiments, the amino acid substitutions are non-conservative substitutions. The data herein indicates that it may be beneficial to substitute acidic amino acid residues with basic amino acid residues. Thus, in certain embodiments the substitutions comprise one or more substitutions of acidic amino acid residues with basic amino acid residues.

The fragment of the polypeptide may be a 15 amino acid fragment of RUCILP1. In some embodiments, the fragment of the polypeptide consists of the amino acid sequence according to SEQ ID NO: 27. In some embodiments, the fragment of the polypeptide consists of the amino acid sequence according to SEQ ID NO: 33. In some embodiments, the fragment of the polypeptide consists of the amino acid sequence according to SEQ ID NO: 34. In some embodiments, the fragment of the polypeptide consists of the amino acid sequence according to SEQ ID NO: 35. In some embodiments, the fragment of the polypeptide consists of the amino acid sequence according to SEQ ID NO: 36. In some embodiments, the fragment of the polypeptide consists of the amino acid sequence according to SEQ ID NO: 37. In some embodiments, the fragment of the polypeptide consists of the amino acid sequence according to SEQ ID NO: 95. In some embodiments, the present disclosure provides a variant of the polypeptide according to any one of SEQ ID NOs: 27, 33, 34, 35, 36, 37 or 95, wherein said variant has 1, 2 or 3 amino acid substitutions as compared to the sequence from which it is derived, i.e. SEQ ID NOs: 27, 33, 34, 35, 36, 37 or 95.

In some embodiments, the present disclosure provides an isolated polypeptide having a length of less than 50 amino acids comprising or consisting of a fragment of SEQ ID NO: 19 (RUCILP1), wherein said fragment is selected from the group consisting of SEQ ID NOs: 27, 33, 34, 35, 36, 37 and 95, and respective variants thereof having between 1 and 3 amino acid substitutions relative to SEQ ID NO: 19, such as 1, 2 or 3 amino acid substitutions relative to SEQ ID NO: 19.

The fragment of the polypeptide may be a 15 amino acid fragment of RUCILP2. In some embodiments, the fragment of the polypeptide consists of the amino acid sequence according to SEQ ID NO: 107. In some embodiments, the fragment of the polypeptide consists of the amino acid sequence according to SEQ ID NO: 108. In some embodiments, the fragment of the polypeptide consists of the amino acid sequence according to SEQ ID NO: 109. In some embodiments, the fragment of the polypeptide consists of the amino acid sequence according to SEQ ID NO: 110. In some embodiments, the fragment of the polypeptide consists of the amino acid sequence according to SEQ ID NO: 111. In some embodiments, the fragment of the polypeptide consists of the amino acid sequence according to SEQ ID NO: 165. In some embodiments, the fragment of the polypeptide consists of the amino acid sequence according to SEQ ID NO: 168. In some embodiments, the present disclosure provides a variant of the polypeptide according to any one of SEQ ID NOs: 107, 108, 109, 110, 111, 165 or 168, wherein said variant has 1, 2 or 3 amino acid substitutions as compared to the sequence from which it is derived, i.e. SEQ ID NOs: 107, 108, 109, 110, 111, 165 or 168.

In some embodiments, the present disclosure provides an isolated polypeptide having a length of less than 50 amino acids comprising or consisting of a fragment of SEQ ID NO: 4 (RUCILP2), wherein said fragment is selected from the group consisting of SEQ ID NOs: 107, 108, 109, 110, 111, 165 and 168, and respective variants thereof having between 1 and 3 amino acid substitutions relative to SEQ ID NO: 4, such as 1, 2 or 3 amino acid substitutions relative to SEQ ID NO: 4.

In some embodiments, the fragment of the polypeptide is a 19 amino acid fragment of RUCILP1, wherein one amino acid has been replaced with an alanine. Said fragment may have increased binding affinity to the integrin αV/β5 receptor compared to an identical fragment, wherein no amino acids have been altered. In some embodiments, the fragment of the polypeptide consists of the amino acid sequence according to SEQ ID NO: 173. In some embodiments, the fragment of the polypeptide consists of the amino acid sequence according to SEQ ID NO: 176. In some embodiments, the fragment of the polypeptide consists of the amino acid sequence according to SEQ ID NO: 181. In some embodiments, the fragment of the polypeptide consists of the amino acid sequence according to SEQ ID NO: 188. In some embodiments, the present disclosure provides a variant of the polypeptide according to any one of SEQ ID NOs: 173, 176, 181 or 188, wherein said variant has 1, 2 or 3 amino acid substitutions as compared to the sequence from which it is derived.

In some embodiments, the present disclosure provides an isolated polypeptide having a length of less than 50 amino acids comprising or consisting of a fragment of a variant of SEQ ID NO: 19 (RUCILP1), wherein said fragment is selected from the group consisting of SEQ ID NOs: 173, 176, 181 and 188, and respective variants thereof having between 1 and 3 amino acid substitutions relative to SEQ ID NO: 19, such as 1, 2 or 3 amino acid substitutions relative to SEQ ID NO: 19.

In some embodiments, the fragment of the polypeptide is a 19 amino acid fragment of RUCILP2, wherein one amino acid has been replaced with an alanine. Said fragment may have increased binding affinity to the integrin αV/β5 receptor compared to an identical fragment, wherein no amino acids have been altered. In some embodiments, the fragment of the polypeptide consists of the amino acid sequence according to SEQ ID NO: 193. In some embodiments, the fragment of the polypeptide consists of the amino acid sequence according to SEQ ID NO: 196. In some embodiments, the fragment of the polypeptide consists of the amino acid sequence according to SEQ ID NO: 201. In some embodiments, the fragment of the polypeptide consists of the amino acid sequence according to SEQ ID NO: 208. In some embodiments, the present disclosure provides a variant of the peptide according to any one of SEQ ID NOs: 193, 196, 201 or 208, wherein said variant has 1, 2 or 3 amino acid substitutions as compared to the sequence from which it is derived.

In some embodiments, the present disclosure provides an isolated polypeptide having a length of less than 50 amino acids comprising or consisting of a fragment of a variant of SEQ ID NO: 4 (RUCILP2), wherein said fragment is selected from the group consisting of SEQ ID NOs: 193, 196, 201 and 208, and respective variants thereof having between 1 and 3 amino acid substitutions relative to SEQ ID NO: 4, such as 1, 2 or 3 amino acid substitutions relative to SEQ ID NO: 4.

In some embodiments, the fragment of the polypeptide is a fragment of RUCILP1. In some embodiments, the fragment of the polypeptide consists of the amino acid sequence according to SEQ ID NO: 210. In some embodiments, the fragment of the polypeptide consists of the amino acid sequence according to SEQ ID NO: 211. In some embodiments, the fragment of the polypeptide consists of the amino acid sequence according to SEQ ID NO: 212. In some embodiments, the fragment of the polypeptide consists of the amino acid sequence according to SEQ ID NO: 213. In some embodiments, the fragment of the polypeptide consists of the amino acid sequence according to SEQ ID NO: 229. In some embodiments, the fragment of the polypeptide consists of the amino acid sequence according to SEQ ID NO: 232. In some embodiments, the fragment of the polypeptide consists of the amino acid sequence according to SEQ ID NO: 233. In some embodiments, the fragment of the polypeptide consists of the amino acid sequence according to SEQ ID NO: 234. In some embodiments, the fragment of the polypeptide consists of the amino acid sequence according to SEQ ID NO: 235. In some embodiments, the present disclosure provides a variant of the peptide according to any one of SEQ ID NOs: 210, 211, 212, 213, 229, 232, 233, 234 or 235, wherein said variant has 1, 2 or 3 amino acid substitutions as compared to the sequence from which it is derived.

In some embodiments, the present disclosure provides an isolated polypeptide having a length of less than 50 amino acids comprising or consisting of a fragment of SEQ ID NO: 19 (RUCILP1), wherein said fragment is selected from the group consisting of SEQ ID NOs: 210, 211, 212, 213, 229, 232, 233, 234 and 235, and respective variants thereof having between 1 and 3 amino acid substitutions relative to SEQ ID NO: 19, such as 1, 2 or 3 amino acid substitutions relative to SEQ ID NO: 19.

In some embodiments, the fragment of the polypeptide is a fragment of RUCILP2. In some embodiments, the fragment of the polypeptide consists of the amino acid sequence according to SEQ ID NO: 243. In some embodiments, the fragment of the polypeptide consists of the amino acid sequence according to SEQ ID NO: 244. In some embodiments, the fragment of the polypeptide consists of the amino acid sequence according to SEQ ID NO: 245. In some embodiments, the fragment of the polypeptide consists of the amino acid sequence according to SEQ ID NO: 246. In some embodiments, the fragment of the polypeptide consists of the amino acid sequence according to SEQ ID NO: 262. In some embodiments, the fragment of the polypeptide consists of the amino acid sequence according to SEQ ID NO: 265. In some embodiments, the fragment of the polypeptide consists of the amino acid sequence according to SEQ ID NO: 266. In some embodiments, the fragment of the polypeptide consists of the amino acid sequence according to SEQ ID NO: 267. In some embodiments, the fragment of the polypeptide consists of the amino acid sequence according to SEQ ID NO: 268. In some embodiments, the present disclosure provides a variant of the peptide according to any one of SEQ ID NOs: 243, 244, 245, 246, 262, 265, 266, 267 or 268, wherein said variant has 1, 2 or 3 amino acid substitutions as compared to the sequence from which it is derived.

In some embodiments, the present disclosure provides an isolated polypeptide having a length of less than 50 amino acids comprising or consisting of a fragment of SEQ ID NO: 4 (RUCILP2), wherein said fragment is selected from the group consisting of SEQ ID NOs: 243, 244, 245, 246, 262, 265, 266, 267 and 268, and respective variants thereof having between 1 and 3 amino acid substitutions relative to SEQ ID NO: 4, such as 1, 2 or 3 amino acid substitutions relative to SEQ ID NO: 4.

The inventors have identified certain amino acids that may be involved in the interaction of RUCILP2 (SEQ ID NO: 4)/21-AABP2 (SEQ ID NO: 5) with the integrin αV/β5 receptor. Particularly the residues at positions 7, 9 and 58 of SEQ ID NO: 4 and positions 5, 6 and 8 of SEQ ID NO: 5 appear to play a role in RUCILP2—integrin αV/β5 receptor and 21-AABP2—integrin αV/β5 receptor interaction, respectively. Thus, in one embodiment, the polypeptide comprises a V at amino acid position 7 of SEQ ID NO: 4, or a conservative substitution thereof such as an M, I, Y, F or L; and/or a E at amino acid position 9 of SEQ ID NO: 4, or a conservative substitution thereof such as a Q, D, K, N, H or R; and/or an E at amino acid position 58 of SEQ ID NO: 4, or a conservative substitution thereof such as a Q, D, K, N, H or R. In one embodiment, the polypeptide comprises a V at amino acid position 7 of SEQ ID NO: 4, or a conservative substitution thereof such as an M, I, Y, F or L; a E at amino acid position 9 of SEQ ID NO: 4, or a conservative substitution thereof such as a Q, D, K, N, H or R; and an E at amino acid position 58 of SEQ ID NO: 4, or a conservative substitution thereof such as a Q, D, K, N, H or R.

In another embodiment, the polypeptide comprises a Y at amino acid position 5 of SEQ ID NO: 5, or a conservative substitution thereof such as an F, W, M, I, V or L; and/or a F at amino acid position 6 of SEQ ID NO: 5, or a conservative substitution thereof such as an M, Y, I, L, W, or V; and/or an E at amino acid position 8 of SEQ ID NO: 5, or a conservative substitution thereof such as a Q, D, K, N, H or R; and/or a N at amino acid position 17 or a conservative substitution thereof, such as a D, S or Q. In one embodiment, the polypeptide comprises a Y at amino acid position 5 of SEQ ID NO: 5, or a conservative substitution thereof such as an F, W, M, I, V or L; a F at amino acid position 6 of SEQ ID NO: 5, or a conservative substitution thereof such as an M, Y, I, L, W, or V; and an E at amino acid position 8 of SEQ ID NO: 5, or a conservative substitution thereof such as a Q, D, K, N, H or R; and/or a N at amino acid position 17 or a conservative substitution thereof, such as a D, S or Q.

The polypeptide disclosed herein may be further modified, for example by the attachment of one or more moieties, thus providing a conjugate of the invention. Such modifications may improve the properties of the polypeptide, hereunder the in vivo stability, membrane permeability, and/or the half-life of the polypeptide. Thus, in one embodiment, the polypeptide comprises one or more moieties conjugated to said polypeptide, optionally wherein the polypeptide and the one or more moieties are conjugated to each other by a linker.

The one or more moieties may be any type of moiety. In one embodiment, the one or more moieties are selected from the group consisting of alkenes, alkyls, aryls, heteroaryls, fatty acids, polyethylene glycol (PEG), saccharides, and polysaccharides. In one embodiment, the alkyl comprises between 1 and 12 carbon atoms, such as between 1 and 6 carbon atoms. In one embodiment, the alkene comprises between 1 and 12 carbon atoms, such as between 1 and 6 carbon atoms. In one embodiment, the fatty acid comprises between 1 and 12 carbon atoms, such as between 1 and 6 carbon atoms.

The polypeptide may form any type of complex, such as dimers and/or multimers. Polypeptide dimers are formed by two polypeptide monomers connected by non-covalent bonds. Polypeptide multimers are formed by more than two polypeptide monomers. Thus, in one embodiment, the polypeptide is a dimer. In another embodiment, the polypeptide is a multimer.

The inventors have shown that the polypeptide as well as fragments and variants thereof presented herein significantly affects various processes on both an organismal and cellular level, including for example cell signaling, peptide secretion, and gene expression. For example, the inventors have shown that the polypeptide disclosed herein binds to at least one type of integrin receptor; the αV/β5 integrin receptor. Thus, in one embodiment, the polypeptide is capable of binding to the αV/β5 integrin receptor.

Integrin receptors, or integrins, are transmembrane receptors which facilitate cell-to-cell and cell-extracellular matrix adhesion. Upon ligand binding, integrins activate signal transduction pathways that mediate cellular signals, such as regulation of the cell cycle, organization of the intracellular cytoskeleton, and movement of new receptors to the cell membrane. Integrins are obligate heterodimers composed of a and § subunits.

The aV class of integrins are receptors which may be present in osteocytes and adipose tissue. The inventors have further shown that the integrin αV/β5 receptor is present in the submucosa of human colon tissue using a duplex RNAscope-based mRNA in situ hybridization array targeting signal dots of integrin αV/β5 receptor (ITGAV and ITGB5 mRNAs).

Adipose tissue, or body fat, is composed mostly of adipocytes. The two main types of adipose tissue are white adipose tissue (WAT) and brown adipose tissue (BAT). WAT is responsible for energy storing, such as storing triglycerides, while BAT is a specialized form of adipose tissue important for adaptive thermogenesis in humans and other mammals. Browning of WAT, also referred to as “beiging”, occurs when adipocytes within WAT depots develop features of BAT. Beige adipocytes take on a multilocular appearance (containing several lipid droplets) and increase expression of several proteins, including uncoupling protein 1 (UCP1). In doing so, these normally energy-storing adipocytes become energy-releasing adipocytes.

The inventors have shown that the polypeptide and the fragments and variants of said polypeptide disclosed herein induces thermogenesis in white adipocytes. In other words, the polypeptide induces browning of WAT. In particular, the polypeptide induces expression of genes involved in thermogenesis and reduces lipogenesis, such as by reducing the expression of genes involved in lipogenesis, in adipocytes. Thus, in one embodiment, the polypeptide induces thermogenesis in white adipocytes, such as for example by inducing expression of genes involved in thermogenesis. In one embodiment, the polypeptide induces mRNA expression in human white preadipocytes of one or more genes selected from the group consisting of Ucp1, Pparγ1, Dio2, Cox2, Cpt1b, and Ebf2, wherein the level of mRNA expression is quantified by q-PCR. In one embodiment, the polypeptide induces mRNA expression in vivo of one or more genes involved in thermogenesis, wherein said genes are selected from the group consisting of UCP1, Dio1, Elovl3, Cidea, Cox2 and Prdm16, wherein the level of mRNA expression is quantified by q-PCR.

In one embodiment, the polypeptide reduces the lipid content of adipocytes, such as for example by reducing expression of genes involved in lipogenesis. In one embodiment, the polypeptide reduces the lipid content in adipocytes, wherein said reduction is measured using oil red O staining. In one embodiment, the polypeptide decreases mRNA expression in vivo of one or more genes involved in lipogenesis, such as Acaca, Scd1 and/or Fasn, wherein the level of mRNA expression is quantified by q-PCR.

Large deposits of WAT is closely linked to obesity and metabolic syndrome. Besides obesity, patients with metabolic syndrome often suffer from high blood pressure, high blood sugar, high serum triglycerides, and low serum high-density lipoprotein (HDL). Metabolic syndrome is also closely related to insulin resistance, T2D, FLD, impaired intestinal barrier junction, and cardiovascular disease.

The inventors have shown that the polypeptide, as well as fragments and variants thereof disclosed herein, act on several factors, such as genes and hormones, related to metabolic syndrome. For example, the inventors have shown that the polypeptide stimulates secretion of glucagon like peptide-1 (GLP-1), insulin, peptide-YY (PYY) and somatostatin and that it induces weight loss and improves glucose tolerance in vivo.

Thus, in one embodiment, the polypeptide stimulates secretion of GLP-1 and glucagon like peptide-2 (GLP-2). In one embodiment, the polypeptide stimulates gut luminal release of GLP-1 and GLP-2. GLP-1 is a peptide hormone capable of promoting insulin secretion in a glucose-dependent manner. GLP-1 further ensures the β cell insulin stores are replenished to prevent exhaustion during secretion, by promoting insulin gene transcription, mRNA stability and biosynthesis. In the stomach, GLP-1 inhibits gastric emptying, acid secretion and motility, which collectively decrease appetite. GLP-1 is secreted in equimolar amounts with glucagon like peptide-2 (GLP-2).

In one embodiment, the polypeptide stimulates secretion of insulin. In one embodiment, the polypeptide stimulates the release of insulin from INS-1 cells. Insulin is a peptide hormone produced by β cells of the pancreatic islets and released into the blood in response to food intake. It is considered the main anabolic hormone of the human body. Insulin regulates the metabolism of carbohydrates, fats and proteins by promoting absorption of glucose form the blood into liver, fat, and skeletal muscle cells.

Glucose production and secretion by the liver is strongly inhibited or absent by high concentrations of insulin in the blood. Decreased or absent insulin activity results in diabetes mellitus, hereunder T2D.

In one embodiment, the polypeptide stimulates secretion of PYY. In one embodiment, the polypeptide stimulates gut luminal release of PYY. PYY is a short peptide released from cells in the ileum and colon in response to feeding. In the blood, gut, and other elements in the periphery, PYY acts to reduce appetite.

In one embodiment, the polypeptide stimulates secretion of somatostatin. In one embodiment, the polypeptide stimulates gut luminal release of somatostatin. Somatostatin is a peptide hormone secreted by delta cells in the digestive system. It decreases the rate of gastric emptying, and suppresses the release of pancreatic hormones such as insulin and glucagon secretion.

In one embodiment, the polypeptide improves glucose tolerance. Glucose tolerance is defined as the ability to dispose of a glucose load. Glucose intolerance, which may be seen in a majority of patients suffering from metabolic syndrome, is defined as an impaired ability to for glucose disposal. Methods for testing glucose tolerance are well known in the art, and include for example challenging a subject with an oral glucose load and measuring the circulating glucose before and after the challenge.

In one embodiment, the polypeptide reduces expression of genes involved in gluconeogenesis. In one embodiment, the polypeptide reduces in HepG2 cells mRNA expression of G6pase and/or Pepck, wherein the mRNA expression levels are quantified using q-PCR.

In one embodiment, the polypeptide enhances the intestinal barrier junction. In one embodiment, the polypeptide increases in Caco-2 cells the mRNA expression of genes involved in intestinal integration, such as Ocln and/or ZO-1, wherein the mRNA expression levels are quantified using q-PCR. The intestinal barrier ensures adequate containment of luminal contents within the intestine, while preserving the ability to absorb nutrients. Dysfunction of the intestinal barrier junction has been implicated in numerous health conditions, including metabolic syndrome, FLD and diabetes, such as T2D.

In one embodiment, the polypeptide induces weight loss. In one embodiment, the polypeptide reduces the fat mass and increases the lean mass of a subject, such as of a mice, a rat or a human. In one embodiment, the polypeptide induces weight loss in a subject suffering from obesity. Obesity is a medical condition in which excess body fat has accumulated to an extent that it may have a negative effect on health. A human is generally considered obese when the body mass index (BMI) is higher than 30 kg/m². Humans with a BMI between 25-30 kg/m²are defined as overweight. As stated above, obesity, and to some extent overweight, is correlated with various diseases and pathological conditions, hereunder cardiovascular disease, musculoskeletal disorders, T2D, and FLD.

Osteocytes are the most commonly found cell in mature bone tissue. Osteocytes synthesize sclerostin, which can increase bone resorption by antagonizing bone formation, and decreasing osteoblastogenesis and osteoblastic activity. Lack of sclerostin expression in bone has been found to be the cause for high bone mass in sclerosteosis. It has further been shown that shown that irisin can modulate bone formation by improving the secretion of sclerostin. In various skeletal disorders, hereunder osteoporosis, the ability to form mature bone tissue is impaired, leading to bone fragility and an increased risk of fractures. The inventors have shown that that the polypeptide disclosed herein stimulates bone formation, and that it increases the cortical thickness of the tibia bone. Thus, in one embodiment, the polypeptide stimulates bone formation, such as for example by stimulating sclerostin expression in osteocytes. In one embodiment, the polypeptide induces mRNA expression of the gene encoding sclerostin in MLO-Y4 (murine long bone osteocyte-Y4) cells, wherein the mRNA expression level is quantified using q-PCR. In another embodiment, the polypeptide increases the cortical thickness of the tibia bone.

In one embodiment, the polypeptide induces cardiomyogenesis. Cardiomyogenesis involves proliferation of bone marrow stem cells that subsequently differentiate into cardiomyocytes. In one embodiment, the polypeptide increases in H9C2 cardiomyoblasts the mRNA expression of FST (Follistatin), wherein the mRNA expression levels are quantified using q-PCR.

In one embodiment, the polypeptide induces myotube formation and myogenesis. In one embodiment, the polypeptide increases the number of formed myotubes. In one embodiment, the polypeptide increases myotube formation in C2C12 myoblasts. In one embodiment, the polypeptide increases the mRNA expression of genes involved in myogenesis, such as Mymk and/or Caveolin-3, wherein the mRNA expression levels are quantified by q-PCR. Myogenesis is the formation of skeletal muscular tissue, i.e. muscle formation. Muscles generally form through the fusion of myoblasts into myotubes. Myogenesis is often impaired in patients with musculoskeletal disorders and muscular dystrophy, such as in patients with Duchenne muscular dystrophy. Impaired myogenesis or muscle weakness has also been reported in ALS, Lambert-Eaton syndrome, myasthenia gravis, and polymyositis.

### Nucleic Acid/Vector/Host Cell

Provided herein is also an isolated polynucleotide encoding the polypeptide presented herein in the section “Polypeptide”.

In one embodiment, the isolated polynucleotide is selected from the group consisting of SEQ ID NOs: 9 to 18, such as SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18. In a preferred embodiment, the polypeptide is selected from the group consisting of SEQ ID NO: 9, SEQ ID NO: 10, SEQ ID NO: 11, and SEQ ID NO: 12.

Also provided is a vector comprising the polynucleotide presented herein. The vector may be any type of vector. In one embodiment, the vector is an expression vector, such as an expression vector selected from the group consisting of bacterial expression vectors, mammalian expression vectors, and insect expression vectors. In one embodiment, the expression is an E. coli expression vector, such as a pGEX-4T-1 expression vector, or an insect expression vector, such as an SF9-insect expression vector.

Further provided is a host cell comprising the polynucleotide presented herein. The host cell may be any type of host cell capable of expressing and secreting the polypeptide encoded by the polynucleotide disclosed herein. In some embodiments, the host cell is a cell naturally present in the human gut microbiota. In one embodiment, the host cell is selected from the group consisting of Lactobacillus, Lactococcus, Escherichia coli, Bacillus subtilis, Pseudomonas putida, Saccharomyces cerevisiae, and Ruminococcus torques. In a preferred embodiment, the host cell is selected from the group consisting of Escherichia coli and Ruminococcus torques.

The polynucleotide and/or vector as described herein may not be naturally comprised in said host cell. Thus, in some embodiments, the polynucleotide comprised in said host cell is heterologous to said host cell. In some embodiments, the vector comprised in said host cell is heterologous to said host cell. In some embodiments, the polynucleotide and/or vector comprised in said host cell are heterologous to said host cell.

In some embodiments, the host cell is a Ruminococcus torques ATCC 27756. In some embodiments, the host cell is a Ruminococcus torques AM22-16. In some embodiments, the host cell is a Ruminococcus torques aa_0143. In some embodiments, the host cell is a Ruminococcus torques 2789STDY5834841.

### Pharmaceutical Composition

In one embodiment, the present invention provides a pharmaceutical composition comprising the polypeptide, the conjugate, the polynucleotide, the vector and/or the host cell as described herein. Said pharmaceutical composition may also comprise a naturally occurring protein comprising said polypeptides, such as the Ruminococcus torques protein RUMTOR_00181 (Uniprot: A5KIY5) as set forth in SEQ ID NO: 21, or vectors or polynucleotides encoding said protein, or host cells comprising said vector or polynucleotide.

The pharmaceutical composition may further comprise one or more pharmaceutically acceptable excipients and/or other additives.

The pharmaceutical composition may further contain one or more additional active ingredients suitable for the treatment of the indications disclosed herein.

### Medical Uses

The data presented herein indicates that the polypeptide as described herein or naturally occurring proteins comprising said polypeptides, such as the Ruminococcus torques protein RUMTOR_00181 (Uniprot: A5KIY5) as set forth in SEQ ID NO: 21, as well as fragments and variants thereof, are effective in the treatment and prevention of metabolic disorders, muscle disorders and injuries, and bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis.

Thus, provided herein is a polypeptide, a conjugate, a polynucleotide, a vector, a host cell, and/or a pharmaceutical composition according to the present invention for use as a medicament. In some embodiments is provided a naturally occurring protein comprising said polypeptides, such as the Ruminococcus torques protein RUMTOR_00181 (Uniprot: A5KIY5) as set forth in SEQ ID NO: 21 or vectors or polynucleotides encoding said protein, or host cells comprising said vector or polynucleotide for use as a medicament.

In one embodiment, the polypeptide, a conjugate, a polynucleotide, a vector, a host cell, and/or a pharmaceutical composition according to the present invention is for use in the treatment of metabolic disorders. In one embodiment, the metabolic disorder is selected from the group consisting of metabolic syndrome, obesity, prediabetes, T2D, and FLD. In some embodiments is provided a naturally occurring protein comprising said polypeptides, such as the Ruminococcus torques protein RUMTOR_00181 (Uniprot: A5KIY5) as set forth in SEQ ID NO: 21 or vectors or polynucleotides encoding said protein, or host cells comprising said vector or polynucleotide for use in the treatment of metabolic disorders, such as a metabolic disorder selected from the group consisting of metabolic syndrome, obesity, prediabetes, T2D, and FLD.

In one embodiment, the polypeptide, a conjugate, a polynucleotide, a vector, a host cell, and/or a pharmaceutical composition according to the present invention is for use in the treatment of muscle disorders and/or muscle injuries. In some embodiments is provided a naturally occurring protein comprising said polypeptides, such as the Ruminococcus torques protein RUMTOR_00181 (Uniprot: A5KIY5) as set forth in SEQ ID NO: 21 or vectors or polynucleotides encoding said polypeptide, or host cells comprising said vector or polynucleotide for use in the treatment of metabolic disorders. In one embodiment, the muscle disorder is selected from the group consisting of muscular dystrophy, Duchenne muscular dystrophy, ALS, Lambert-Eaton syndrome, myasthenia gravis, polymyositis, and peripheral neuropathy.

In one embodiment, the polypeptide, a conjugate, a polynucleotide, a vector, a host cell, and/or a pharmaceutical composition according to the present invention is for use in the treatment of bone disorders. In some embodiments is provided a naturally occurring protein comprising said polypeptides, such as the Ruminococcus torques protein RUMTOR_00181 (Uniprot: A5KIY5) as set forth in SEQ ID NO: 21 or vectors or polynucleotides encoding said polypeptide, or host cells comprising said vector or polynucleotide for use in the treatment of bone disorders. In a preferred embodiment, the bone disorder is selected from the group consisting of osteoporosis, osteogenesis imperfect, and osteopetrosis.

Thus, provided herein is a polypeptide, a conjugate, a polynucleotide, a vector, a host cell, and/or a pharmaceutical composition for use in the treatment and/or prevention of metabolic disorders, muscle disorders and injuries, and/or bone disorders. In some embodiments is provided a naturally occurring protein comprising said polypeptides, such as the Ruminococcus torques protein RUMTOR_00181 (Uniprot: A5KIY5) as set forth in SEQ ID NO: 21 or vectors or polynucleotides encoding said polypeptide, or host cells comprising said vector or polynucleotide for use in the treatment and/or prevention of metabolic disorders, muscle disorders and injuries, and/or bone disorders.

Further provided herein is a polypeptide, a conjugate, a polynucleotide, a vector, a host cell, and/or a pharmaceutical composition according to the present invention for use in the treatment and/or prevention of diseases, disorders and conditions selected from the group consisting of metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis. In some embodiments is provided a naturally occurring protein comprising said polypeptides, such as the Ruminococcus torques protein RUMTOR_00181 (Uniprot: A5KIY5) as set forth in SEQ ID NO: 21 or vectors or polynucleotides encoding said polypeptide, or host cells comprising said vector or polynucleotide for use in the treatment and/or prevention of diseases, disorders and conditions selected from the group consisting of metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis.

Also provided herein is the use of the polypeptide, the conjugate, the polynucleotide, the vector, the host, and/or the pharmaceutical composition, in the manufacture of a medicament for treatment of metabolic disorders, muscle disorders and injuries, and/or bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis. In some embodiments is provided the use of a naturally occurring protein comprising said polypeptides, such as the Ruminococcus torques protein RUMTOR_00181 (Uniprot: A5KIY5) as set forth in SEQ ID NO: 21 or vectors or polynucleotides encoding said polypeptide, or host cells comprising said vector or polynucleotide in the manufacture of a medicament for treatment of metabolic disorders, muscle disorders and injuries, and/or bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis.

Further provided herein is a method for the treatment of metabolic disorders, muscle disorders and injuries, and/or bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis, wherein the method comprises administering the polypeptide, the conjugate, the polynucleotide, the vector, the host, and/or the pharmaceutical composition as described herein to an individual in need thereof. In some embodiments is provided a method for the treatment of metabolic disorders, muscle disorders and injuries, and/or bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis, wherein the method comprises administering a naturally occurring protein comprising said polypeptides, such as the Ruminococcus torques protein RUMTOR_00181 (Uniprot: A5KIY5) as set forth in SEQ ID NO: 21 or vectors or polynucleotides encoding said polypeptide, or host cells comprising said vector or polynucleotide to an individual in need thereof.

The polypeptide, the conjugate, the polynucleotide, the vector, the host, and/or the pharmaceutical composition is administered in a therapeutically effective amount. Similarly, the naturally occurring protein comprising said polypeptides, such as the Ruminococcus torques protein RUMTOR_00181 (Uniprot: A5KIY5) as set forth in SEQ ID NO: 21 or vectors or polynucleotides encoding said polypeptide, or host cells comprising said vector or polynucleotide are administered in a therapeutically effective amounts.

In one embodiment, the individual or subject is a mammal, preferably a human being.

In one embodiment, the present disclosure provides Ruminococcus torques for use in the treatment of metabolic disorders, muscle disorders and injuries, and/or bone disorders, such as for example metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect, and osteopetrosis.

### Probiotic or Live Biopharmaceutical Product Uses

The data presented herein indicates that the polypeptides as described herein, the naturally occurring proteins comprising said polypeptides, such as Ruminococcus torques RUMTOR_00181 (Uniprot: A5KIY5), as well as fragments and variants thereof, are useful when comprised in a probiotic or in a Live Biopharmaceutical Product (LBP), or when administered as a dietary composition.

In some aspects, is thus provided a dietary composition comprising
a) the polypeptide or conjugate as described elsewhere herein;
b) a RUMTOR_00181 polypeptide comprising or consisting of
   i. the polypeptide according to SEQ ID NO: 21; or
   ii. a variant of SEQ ID NO: 21 with at least 85%, such as at least 90%, such as at least 95%, such as at least 98% sequence identity thereto;
c) the polynucleotide as described elsewhere herein;
d) a polynucleotide encoding said RUMTOR_00181 polypeptide;
e) the vector according as described elsewhere herein,
f) a vector comprising the polynucleotide encoding said RUMTOR_00181 polypeptide; and/or
g) the host cell according to any one of items 24 to 26; and/or
h) a host cell comprising
   i. a polynucleotide encoding said RUMTOR_00181 polypeptide; and/or
   ii. a vector comprising the polynucleotide encoding said RUMTOR_00181 polypeptide, wherein the dietary composition optionally further comprises one or more of prebiotics, probiotics, synbiotics, proteins, lipids, carbohydrates, vitamins, fibers, and/or nutrients, such as dietary minerals.

In some aspects is also provided a host cell comprising
a) the polypeptide or conjugate as described elsewhere herein, and/or a RUMTOR_00181 polypeptide comprising or consisting of
   i. the polypeptide according to SEQ ID NO: 21; or
   ii. a variant of SEQ ID NO: 21 with at least 85%, such as at least 90%, such as at least 95%, such as at least 98% sequence identity thereto;
b) the polynucleotide as described elsewhere herein and/or a polynucleotide encoding said RUMTOR_00181 polypeptide; and/or
c) the vector as described elsewhere herein and/or or a vector comprising the polynucleotide encoding said RUMTOR_00181 polypeptide;
for use as a probiotic or as a Live Biopharmaceutical Product (LBP).

In another aspect is provided the use of a host cell comprising
a) the polypeptide or conjugate as described elsewhere herein, and/or a RUMTOR_00181 polypeptide comprising or consisting of
   i. the polypeptide according to SEQ ID NO: 21; or
   ii. a variant of SEQ ID NO: 21 with at least 85%, such as at least 90%, such as at least 95%, such as at least 98% sequence identity thereto;
b) the polynucleotide as described elsewhere herein and/or a polynucleotide encoding said RUMTOR_00181 polypeptide; and/or
c) the vector as described elsewhere herein and/or or a vector comprising the polynucleotide encoding said RUMTOR_00181 polypeptide;
as a probiotic or as a Live Biopharmaceutical Product (LBP).

It is also an aspect to provide the use of the polypeptide or conjugate as described elsewhere herein, or a RUMTOR_00181 polypeptide comprising or consisting of
i. the polypeptide according to SEQ ID NO: 21; or
ii. a variant of SEQ ID NO: 21 with at least 85%, such as at least 90%, such as at least 95%, such as at least 98% sequence identity thereto;
the polynucleotide as described elsewhere herein, or a polynucleotide encoding said RUMTOR_00181 polypeptide; the vector as described elsewhere herein, or a vector comprising the polynucleotide encoding said RUMTOR_00181 polypeptide;
the host cell as described elsewhere herein, or a host cell comprising
i. a polynucleotide encoding said RUMTOR_00181 polypeptide; or
ii. a vector comprising the polynucleotide encoding said RUMTOR_00181 polypeptide;
as a food ingredient or as a food or beverage additive.

In some embodiments, said variant of SEQ ID NO: 21 has at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to SEQ ID NO: 21.

Administration of said probiotic, Live Biopharmaceutical Product (LBP) or dietary composition to a subject has a range of health benefits. In some embodiments, administration of said probiotic, LBP or dietary composition results in the effects as described elsewhere herein for the administration of the isolated polypeptide.

In some embodiments, administration of said probiotic, LBP or dietary composition results in reduced body fat mass, such as by reduction in the adipocyte size of white adipose tissue. In some embodiments, administration of said probiotic, LBP or dietary composition results in increased lean body mass.

In some embodiments, administration of said probiotic, LBP or dietary composition results in increased thermogenesis in fat tissue, such as increased thermogenesis as measured by increased expression of mRNA encoding thermogenic markers, e.g. Ucp1, Cidea, or Dio2 or corresponding mRNA. In some embodiments, administration of said probiotic, LBP or dietary composition results in decreased adipose lipogenesis, such as decreased adipose lipogenesis as measured by reduced expression of mRNA encoding genes involved in adipose lipogenesis, e.g. Fasn, Scd1, and Acaca or corresponding genes. In some embodiments administration of said probiotic, LBP or dietary composition results in an increased protein level of UCP1 in fat tissue.

In some embodiments, administration of said probiotic, LBP or dietary composition results in improved glucose tolerance. In some embodiments, administration of said probiotic, LBP or dietary composition results in increased bone mass.

### EXAMPLES

### Example 1—Discovery and Characterization of a Novel Polypeptide Hormone Released from Common Commensal Bacterial Strains in the Human Gut Microbiome

### Main Results

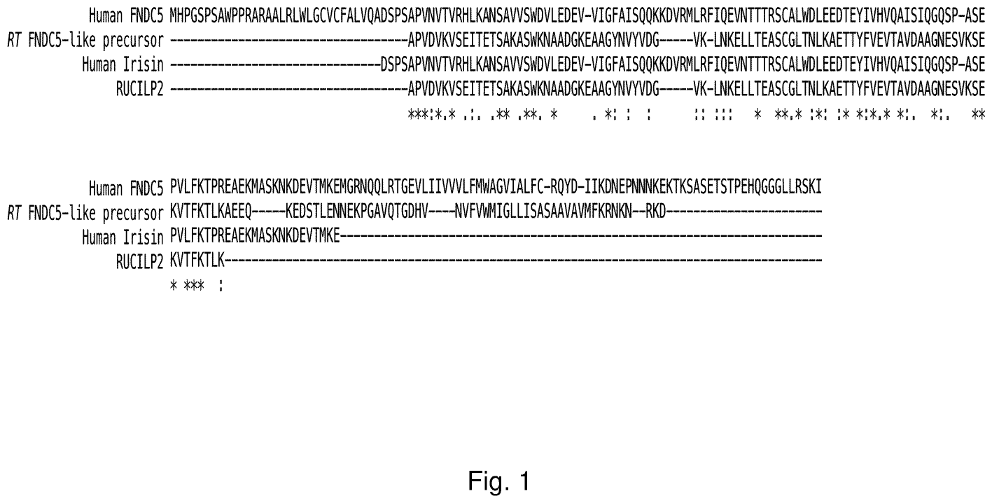

Applying an in silico (alignment) approach, we searched about 5,600 full prokaryotic genomes for partial sequence homology with 118 mammalian polypeptide hormones, metabolism-related cytokines, and neuropeptides. We identified the 118 peptides and cytokines by searching public available scientific literature in week 4 of 2019. One alignment hit predicted the presence of a bacterial pre-polypeptide with a relatively high homology to a known human precursor protein, Fibronectin Type III Domain Containing Protein 5 (FNDC5)⁶ (FIG. 1). The human FNDC5 of 212 amino acids including signal peptide is primarily expressed in skeletal muscle where it following acute exercise is cleaved to the myokine, Irisin that consists of 112 amino acids⁷.

The bacterial FNDC5-like protein contains 142 amino acids, of which 66% of the amino acids showed similarity with human FNDC5 (calculated by dividing the number of amino acids in bacterial FNDC5-like protein that has identical and similar chemical structures to human FNDC5, by the length of bacterial FNDC5-like protein and multiplying by 100%). The bacterial FNDC5-like protein is expressed by four strains of the commensal Ruminococcus torques (RT) species that with its 20 known strains is both prevalent and highly abundant (up to 10%) in the human gut microbiotas. The four RT strains having the gene encoding the FNDC5-like protein were predicted to synthesize an 87 amino acid polypeptide that has an overall 64% amino acid sequence similarity and 32% amino acid identity to human irisin (calculated by dividing the number of amino acids in bacterial FNDC5-like protein that has identical and similar chemical structures to human irisin, by the length of bacterial FNDC5-like protein and multiplying by 100%, FIG. 1). The enzyme(s) cleaving the human FNDC5 and the bacterial FNDC5-like protein is unknown⁷. We coined the enzymatically cleaved bacterial fragment FNDC5-like protein for RUminococCus torques Irisin-Like Peptide 2 (RUCILP2).

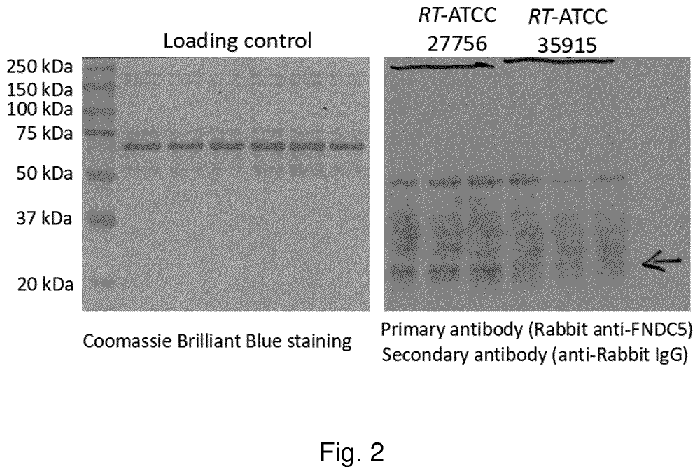

In bacteria culture experiments of one of the four RT strains, RT-ATCC 27756, carrying the FNDC5-like sequence of interest, and of a control strain without the specific sequence, RT-ATCC 35915, we documented that the RT strain carrying the sequence for synthesis of RUCILP2 releases the polypeptide to the culture medium (FIG. 2).

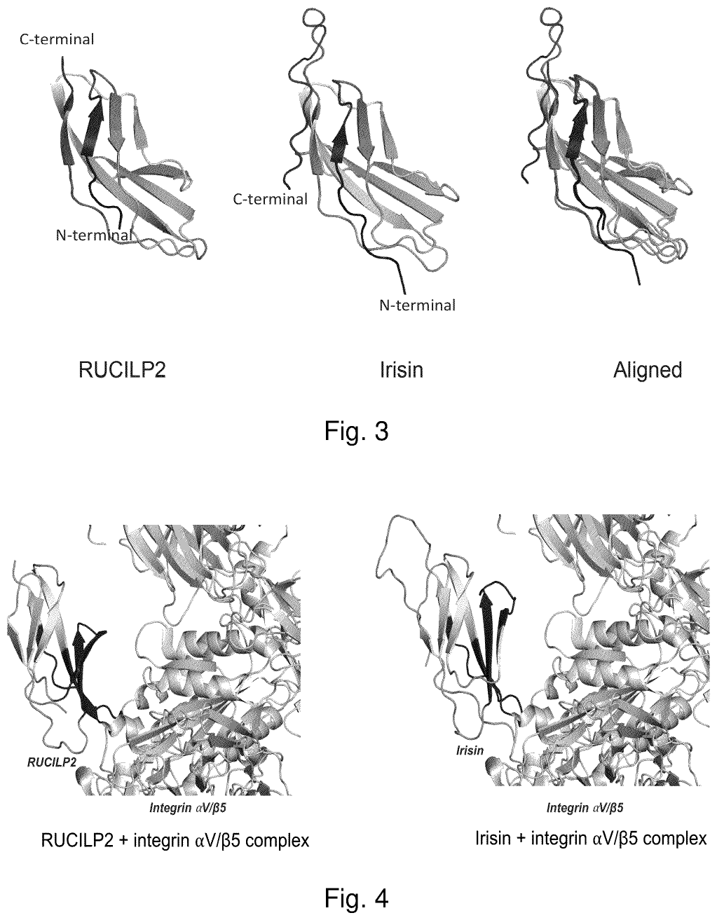

Applying a docking model of the interaction between irisin and the αV/β5 (ITGAV/ITGB5) integrin receptor⁹, we assessed the putative 3D structure of RUCILP2 (FIG. 3). The estimated free energy of RUCILP2 receptor binding is −1.43 kcal/mol, suggesting a high binding affinity between RUCILP2 and the αV/β5 integrin receptor, which is ubiquitously expressed (FIG. 4).

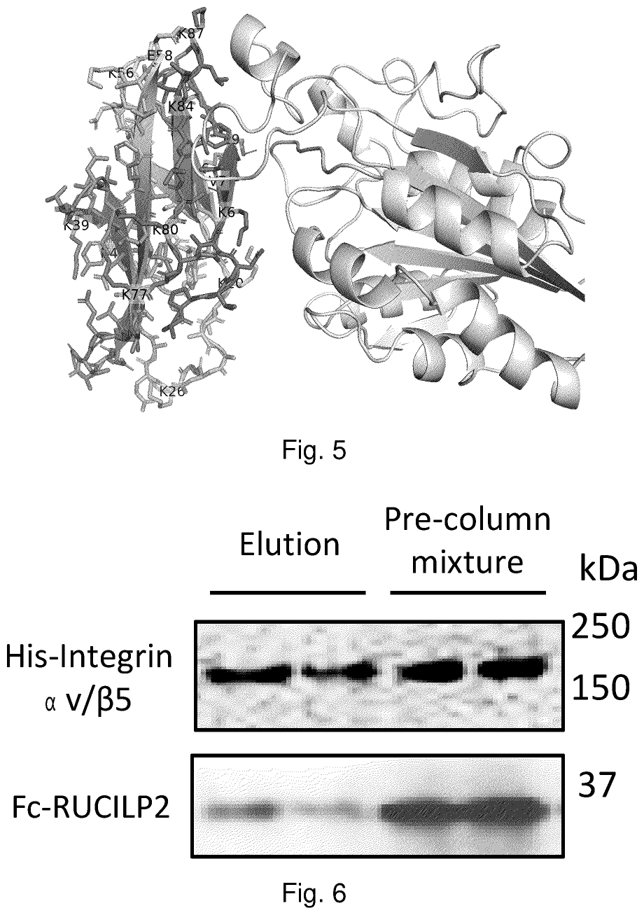

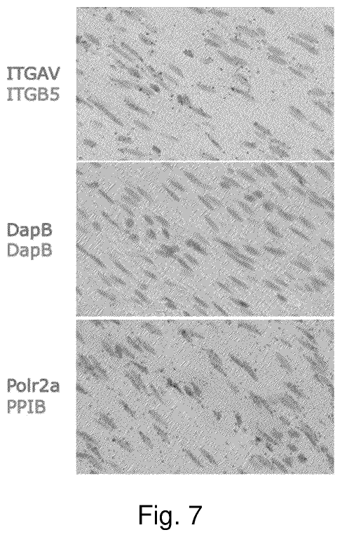

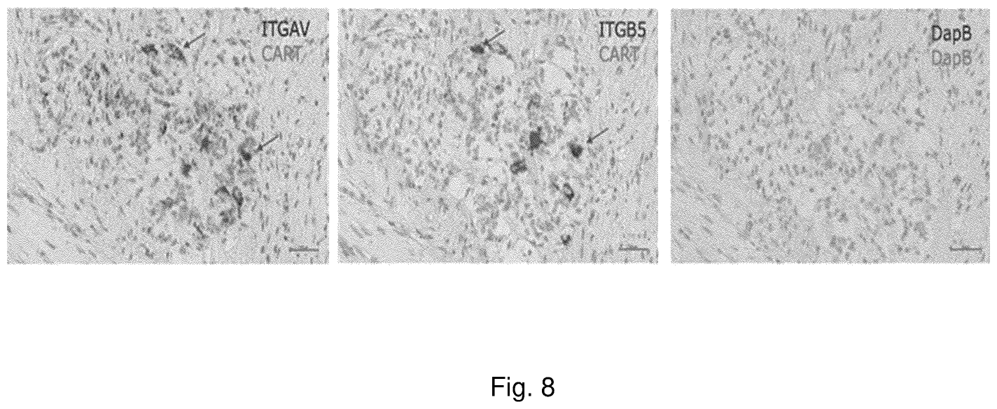

Applying the ZDOCK prediction tool and PyMOL program, we predicted amino acids V7, E9 and E58 of RUCILP2 as the binding sites to the αV/β5 integrin receptor (FIG. 5). With recombinant RUCILP2, we have experimentally shown binding of the ligand to the αV/β5 integrin receptor (FIG. 6). In addition to the in silico analysis, presence of αV/β5 integrin receptor in human colon was visualized by RNAscope-based mRNA in situ hybridization and immunostaining (FIG. 7 and FIG. 8).

### Materials & Methods

### Bioinformatics Analyses:

The reference prokaryotic genome database was downloaded from the NCBI (ftp://ftp.ncbi.nim.nih.gov/blast/db) and was searched for the 118 peptide and cytokine amino acid sequences from Uniprot (https://www.uniprot.org/) reported in Supplementary table with a threshold e-value ≤0.1 using tBLASTn. In alignment analyses, we showed that RUCILP2 was predicted to be synthesized from the FNDC5-like precursor as an 87 amino acids protein that has an overall 64% amino acid sequence homology, and 32% amino acid sequence identity, to irisin. The multiple sequence alignments of amino acids in human FNDC5, FNDC5-like bacterial protein, human irisin, and RUCILP2 were performed using open access tool Clustal Omega (https://www.ebi.ac.uk/Tools/msa/clustalo/) to determine the number of the identical and conserved residues. The open access website tool I-TASSER (https://zhanglab.ccmb.med.umich.edu/I-TASSER/) was used to predict 3D structures of RUCILP2. The binding abilities of RUCILP22 and irisin to the integrin αV/β5 receptor was assessed by ZDOCK (https://zdock.umassmed.edu/) computational analysis. The final complex structures of docking models were demonstrated by PyMOL (v2.1.1). The direct binding interactions within the complex were visualized by PyMOL (v2.1.1).

### Experimental Validation:

### (1) The Release of RUCILP2 in Cultured RT Strain

In culture experiments of one of the RT strains, RT-ATCC 27756 carrying the FNDC5-like protein sequence of interest, and of a control strain without the specific sequence, RT-ATCC 35915, we documented that the RT strain carrying the sequence for synthesis of RUCILP2 released the peptide to the culture medium. Detailed experimental protocols were as below:
(1) Add 100 μl of bacterial protein extraction reagent (Thermo Scientific, 90080, containing lysozyme and DNase I (Fisher Scientific, 181610), and supplemented with 1 mM Dithiothreitol (Sigma, 10197777001), 0.5 mM phenylmethylsulfonyl fluoride (Sigma, 10837091001), and phosphatase inhibitor cocktail (Fisher Scientific, 78440) for every 1 ml of resuspended cell culture and mix by pipetting up and down.
(2) Incubate cells for 15 minutes at room temperature.
(3) Centrifuge for 10 min at 13000 revolutions per minute (rpm) and get the supernatant.
(4) Pierce Rapid Gold BCA Protein Assay for each standard or bacterial protein lysate sample, 20 μL was dispensed in replicate into a 96-well microplate. The Pierce Rapid Gold BCA Protein Assay (Fisher Scientific, A53225) working reagent was prepared by mixing 50 parts of reagent A and 1 part of reagent B, and 200 μl of the working reagent were added to each well with a multichannel pipette and mixed thoroughly on a plate shaker for 30 seconds. The plate was incubated at room temperature for 5 minutes, and absorbance was then detected at 480 nm on a Thermo Scientific™ Multiskan™ GO Microplate Spectrophotometer. Unknown protein concentrations were determined using a standard curve.
(5) Protein extracts (30 μg) were incubated at 98° C. for 10 minutes before resolved by sodium dodecyl sulphate-polyacrylamide (SDS-PAGE) gel, transferred onto polyvinylidene fluoride (PVDF) membranes, blocked with 5% skim milk, and incubated with rabbit anti-FNDC5 antibody (abcam, ab131390) overnight.
(6) Membranes were incubated with anti-rabbit immunoglobulin G (IgG) secondary antibodies (Fisher Scientific, G21234) and visualized by enhanced chemiluminescence. Equal loading was confirmed using Coomassie brilliant blue staining of PVDF membranes (PVDF, Millipore, IPFL85R).

### (2) Co-Immunoprecipitation on a Nickel Ion Column to Validate Binding of RUCILP2 to the αV/β5 Integrin Receptor Complex

Step 1: Binding of integrin and RUCILP2. One hundred nM FC-tagged RUCILP2 was incubated with 5 nM of the indicated his-tag integrin avb5 in a final volume of 500ul in 1.5 ml Protein LoBind Tubes (Eppendorf, 022431081) for 5/30/90 minutes at room temperature under rotation.
Step 2: After rotation, Ni spin column (ThermoFisher Scientific, R901-01) was applied to immunoprecipitate integrins. In details, Add 500 μl of FC-RUCILP2-integrin-His protein complex to the column. Mix end-over-end at 4° C. for 15 minutes. Wash the column for twice followed by two times elution and save the eluted samples.
Step 3: Extra elution: Add 200 μl of SDS-PAGE loading buffer to the column, pipette up and down and remove an aliquot of the resin from the spin column. Incubate at 70° C. for 5 minutes to release any protein that remains on the resin following elution. Load and analyze proteins by SDS-PAGE. Most proteins, regardless of whether they bind to the nickel or to the agarose-bead itself, will be recovered by this procedure.
Step 4: Samples in each procedure were incubate at 70° C. for 10 minutes to dissociate RUCILP2 and integrin.
Step 5: Analyze the by SDS-PAGE. Load and analyze proteins mix (FC-RUCILP2-integrin-His protein complex (before loaded to the column), flow through column, washes and eluates) by SDS-PAGE. Precipitated integrin was detected by immuno-blot analysis against his tag. Co-precipitated RUCILP2 was detected by immunoblot analysis against FC-tag. Each sample will be loaded to both of the two gels but be tested by primary antibody of anti his-tag integrin and anti-FC integrin αV/β5, respectively.
   (3) Visualization of Integrin αV/β5 (ITGAV/ITGB5) Receptor in Human Colon Tissue Samples Using RNAscope-Based mRNA In Situ Hybridization (ISH) and Immunostaining

Samples: The ITGAV/ITGB5 mRNA ISH analyses were performed on three paraffin samples with normal human colon tissue obtained from BioIVT. A section was stained with Haemotoxylin and Eosin to confirm that the tissue contained both colon mucosa and the colon wall with presence of ganglion/nerve cells.

Performance of the duplex assay: The experimental setup included both positive and negative control probe sets. The two target mRNAs were stained as red and green signal dots, that, when appearing in excess, become a more diffuse precipitate. The ISH signal dots were visualized as red stain (ITGAV) and green stain (ITGB5), respectively. RNAscope probes with indication of detection channel and associated chromogen.

| | | | ZZ | | |
|---|---|---|---|---|---|
| | RNAscope | Target | pairs | | |
| Probe | Target | Sequence | (n) | Channel | Chromogen |
| Integrin | ITGAV | 460-1419 | 20 | 2 | Fast Red |
| alpha V | | | | | |
| Integrin | ITGB5 | 1048-2077 | 20 | 1 | Teal (green) |
| beta 5 | | | | | |
| Positive | PPIB mRNA | 139-989 | 16 | 1 | Teal (green) |
| control | | | | | |
| Positive | Polr2a mRNA | 2514-3433 | 20 | 2 | Fast Red |
| control | | | | | |
| Negative | DapB mRNA | NA | NA | 1 | Teal (green) |
| control | (bacterial) | | | | |
| Negative | DapB mRNA | NA | NA | 2 | Fast Red |
| control | (bacterial) | | | | |

Images were acquired using a 20× objective with a Zeiss AxioScan. Representative areas were selected for presentation.

### Example 2—Effects of RUCILP2: Studies in Cells (In Vitro) and Rodents (In Vivo)

### Main Results

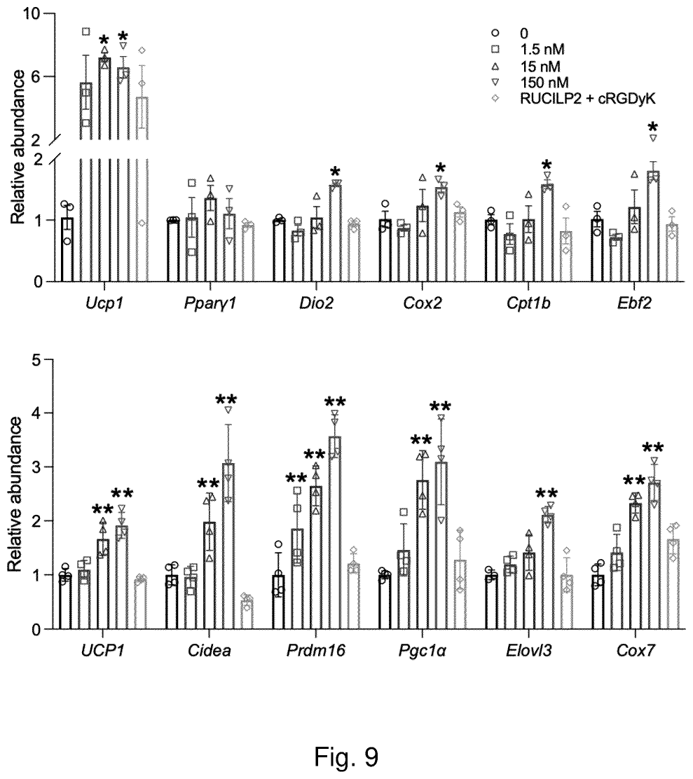

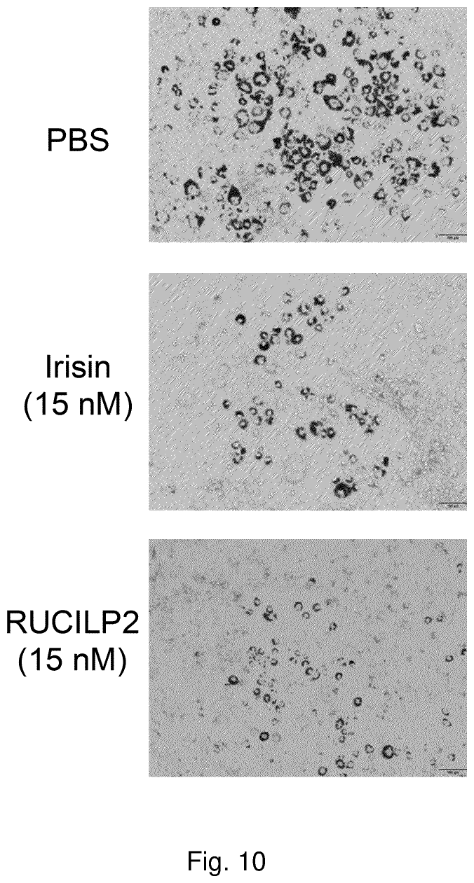

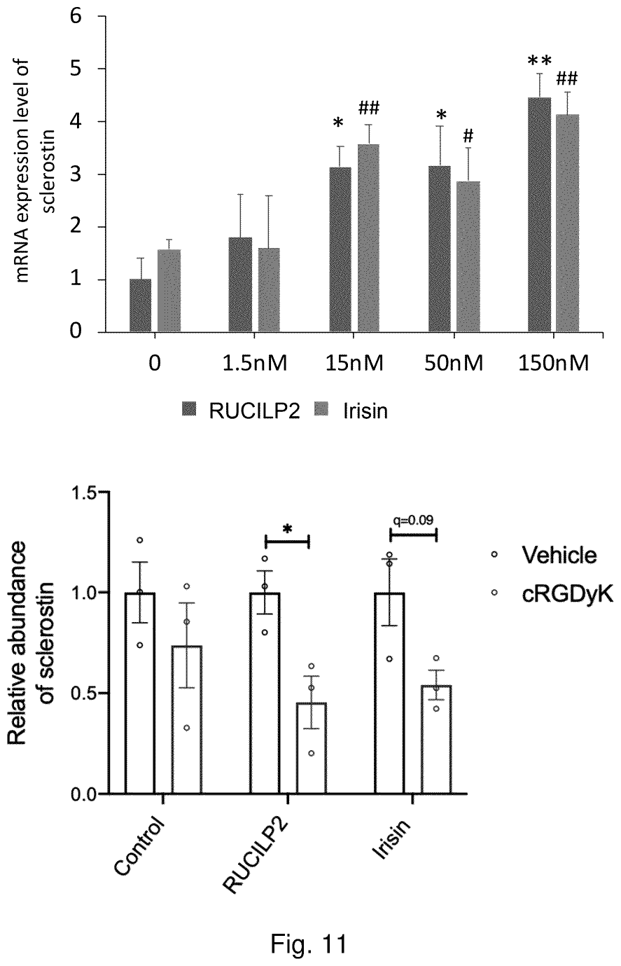

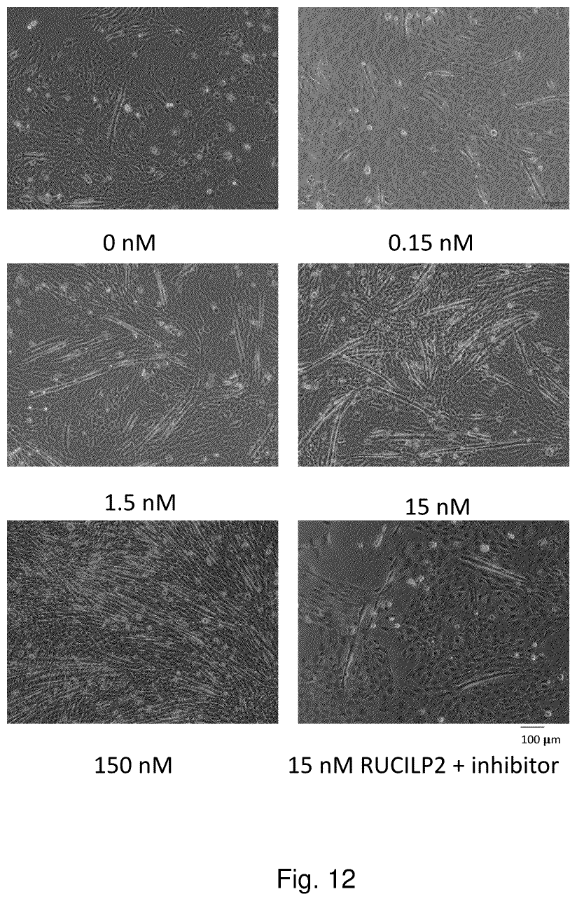

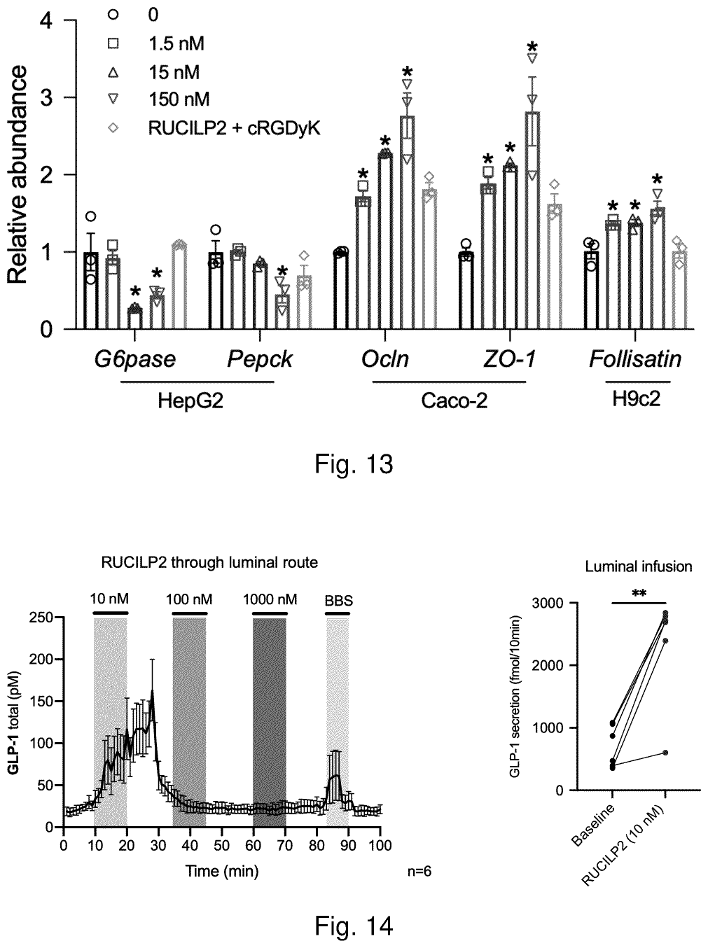

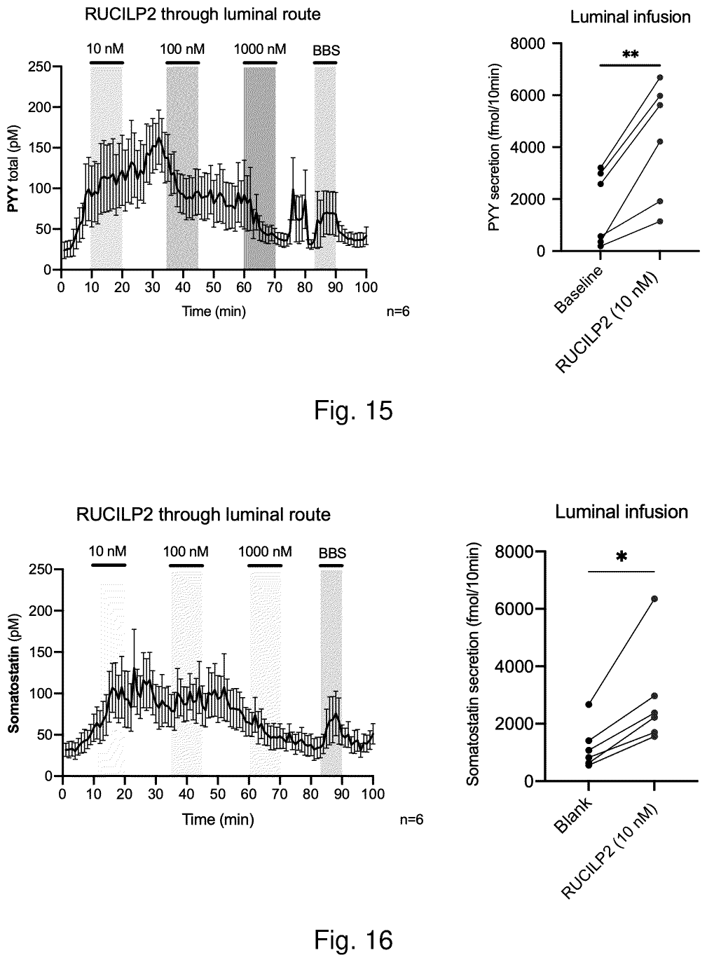

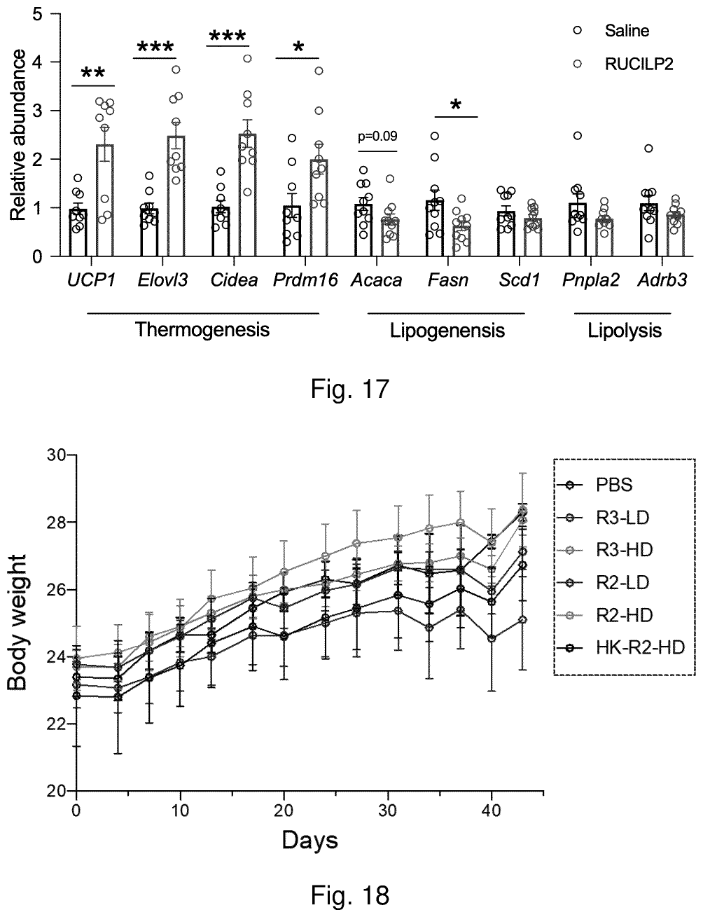

In both cellular and in mice experiments, we provided evidence of metabolic effects of recombinant RUCILP2. Thus, we found that RUCILP2 and irisin in equimolar concentration (cellular studies) or dose (in vivo studies) had similar effects on both expression of key genes of thermogenesis and browning in human and murine pre-adipocytes (FIG. 9) in a dose-dependent manner. RUCILP2 inhibited expression of genes regulating lipogenesis in adipocytes (FIG. 10 and FIG. 17). RUCILP2 produced marked stimulatory effects on bone formation (FIG. 11) and myogenesis (FIG. 12). In liver cells, the hormone inhibits the expression of genes regulating gluconeogenesis (FIG. 13). In addition, RUCILP2 increased expression of genes involved in intestinal barrier function of gut epithelial cells as well as markers of cardiomyogenesis (FIG. 13). Cellular effects of RUCILP2 were blocked by pre-treatment with CycloRGDyK, a nonspecific inhibitor of the integrin receptor. In live rat colon perfusion experiments, RUCILP2 exhibited stimulatory effects on gut luminal release of Glucagon-like peptide-1 (GLP-1, FIG. 14), Glucagon-like-peptide-2 (GLP-2), Peptide YY (PYY, FIG. 15) and somatostatin (FIG. 16) through the luminal infusion.

### Materials & Methods

(1) Recombinant Synthesis of 6-His-Tagged RUCILP2 in Escherichia coli

Target DNA sequence of the 87 amino acid RUCILP2 polypeptide was codon-optimized for Escherichia coli and synthesized. The synthesized sequence was cloned into vector pET-30a (+) with 6-His-tag for protein expression in E. coli strain BL21 star (DE3) that was transformed with recombinant plasmid. A single colony was inoculated into Terrific Broth (TB) medium containing related antibiotic; the culture was incubated at 37° C. at 200 rpm and then induced with isopropyl β-D-1-thiogalactopyranoside (IPTG). SDS-PAGE was used to monitor the expression. Recombinant BL21 star (DE3) stored in glycerol was inoculated into TB medium containing related antibiotic and cultured at 37° C. When the OD 600 reached about 1.2, the cell culture was induced with IPTG at 37° C./4h. Cells were harvested by centrifugation. Cell pellets were resuspended with lysis buffer followed by sonication. The supernatant after centrifugation was kept for future purification. Target protein was obtained by one-step purification using Ni column. Target protein was kept in 50 mM Tris-HCl, 150 mM NaCl, 10% Glycerol, pH 8.0 followed by sterilized by 0.22 μm filter before stored in aliquots. The concentration was determined by a bicinchoninic acid (BCA) TM protein assay with bovine serum albumin (BSA) as standard. The protein purity and molecular weight were determined by standard SDS-PAGE along with Western blot confirmation. The protein was diluted in sterilized phosphate-buffered saline (PBS) to use in cell culture experiments and in vivo injection.

### (2) Effects of Recombinant RUCILP2 on Human Visceral White Pre-Adipocytes

Human white pre-adipocytes were cultured until 80% confluent and switched into differentiation media (with 0.3 ml/ml fetal calf serum, 8 μg/ml d-biotin, 0.5 μg/ml insulin, 400 ng/ml dexamethasone). The differentiation process to mature adipocytes was completed after 12-14 days. The treatment of RUCILP2 and irisin started from the third day of differentiation. For integrin complex inhibition, cells were treated with 10 μM of CycloRGDyK (Selleckchem, #S7844) for 10 minutes and washed with PBS before treated with RUCILP2 and irisin, respectively. Cells were harvested after 14 days of differentiation and thermogenesis gene were quantified by quantitative polymerase chain reaction (q-PCR).

### (3) Effects of Recombinant RUCILP2 on Murine Inguinal Pre-Adipocytes

Inguinal fat tissues from 10-week-old wild-type C57BL/6J male mice were dissected and washed with Dulbecco's Modified Eagle Medium (D-MEM) containing 1% penicillin-streptomycin (P/S) solution, minced and digested for 1 hour at 37° C. in D-MEM (1% P/S) containing 2% BSA, 0.2% collagenase type 1. Digested tissue was then centrifugated at 400 g for 5 min at room temperature. Pellet was resuspended by 10 ml of D-MEM containing 10% FBS and 1% P/S and filtered through a 200 m cell strainer. Inguinal stromal vascular cells were split onto type I collagen-coated coated 12-well plates and grown to confluence before inducing to differentiation by treatment with 1 μM rosiglitazone, 86 nM insulin, 0.1 μM dexamethasone, 1 nM triiodo-L-thyronine (T3), and 250 μM methyl isobutylxanthine.

Two days after induction, cells were switched to inducing medium in the presence of 15 nM recombinant RUCILP2, or commercial recombinant irisin (Sigma, #SRP8039-10UG and Phoenix pharmaceuticals, #067-29A), or saline for two days. After that, cells were maintained in 86 nM insulin and 1 nM T3 in the presence of indicated concentrations of recombinant RUCILP2 or commercial recombinant irisin, or saline for four days with medium change every other day. For inhibition of the integrin complex, cells were treated with 10 μM of cRGDyK for 10 minutes before treatment with recombinant RUCILP2 or commercial recombinant irisin or saline every other day during 6 days of differentiation. Cells will be harvested for qRT-PCR analysis as described in protocol for gene expression analysis.

Oil red O staining of lipids in adipocytes was performed according to the protocol below:
(1) Fixation—Remove the medium and gently wash the cells twice with PBS. Add formalin (10%) to the cells and incubate for 30 minutes
(2) Discard the formalin and wash the cells twice using sterile water. Add 60% isopropanol to the cells and incubate for 5 minutes
(3) Discard 60% isopropanol and cover the cells evenly with Oil Red O Working Solution. Rotate the plate or dish, and incubate for 15 minutes
(4) Discard the Oil Red O solution and wash the cells four times with water until no excess stain is seen
(5) Add hematoxylin to the cells and incubate for 1 minute. Discard hematoxylin and wash the cells four times with water
(6) Cover the cells with sterile water and view under microscope. Lipid droplets appear red, and nuclei appear blue

### (4) Effects of Recombinant RUCILP2 on Murine Long Bone Osteocyte-Y4 (MLO-Y4) Cell Line

MLO-Y4 cells were donated by Prof Moustapha Kassem from the University of Southern Denmark. The cells were seeded on type I collagen-coated 6-well plates under Minimum Essential Medium (α-MEM from Fisher Scientific, #15430584), supplemented with 2.5% Fetal Bovine Serum (FBS from Fisher Scientific, #11550356), 2.5% calf serum (Hyclone, SH30072.03), and 1% Penicillin-Streptomycin (Fisher Scientific, #11548876). Cell cultures were maintained in a humidified chamber with 5% CO₂ at 37° C., and culture media were changed every 2-3 days.

At 60% confluence, medium was switched to FreeStyle293 Expression medium after washing with warm PBS. After 4 hours incubation, the cells were treated with recombinant RUCILP2 or commercial recombinant irisin (Sigma, #SRP8039-10UG and Phoenix pharmaceuticals, #067-29A) or saline for 24 h. For integrin inhibitor treatment, cells were treated with 10 μM of CycloRGDyK (Selleckchem, #S7844) for 10 minutes and washed with PBS before treated with recombinant RUCILP2 or commercial recombinant irisin or saline. After treatments, MLO-Y4 cells were harvested for qRT-PCR analysis of the mRNA level of sclerostin as described in gene expression analysis.

### (5) Effects of Recombinant RUCILP2 on Immortalized Mouse Myoblasts, the C2C12 Cell Line

C2C12 cells were seeded on 12-well plates under DMEM/F-12 medium (Dulbecco's Modified Eagle Medium/Nutrient Mixture F-12, Fisher Scientific, #11524436), supplemented with 10% FBS (Fetal Bovine Serum, Fisher Scientific, #11550356) and 1% Penicillin-Streptomycin (Fisher Scientific, #11548876). Cell cultures were maintained in a humidified chamber with 5% CO₂ at 37° C., and culture media were changed every 2-3 days.

At approximately 80% confluence, 10% fetal bovine serum was replaced with 2% horse serum to induce C2C12 myoblast cells to differentiate into myotubes. Twenty-four hours later (day 1 of the differentiation), the cells were treated with recombinant RUCILP2 or commercial recombinant irisin (Sigma, #SRP8039-10UG and Phoenix pharmaceuticals, #067-29A) or saline. For integrin inhibitor treatment, cells were treated with 10 μM of CycloRGDyK (Selleckchem, #S7844) for 10 minutes and washed with PBS before treated with recombinant RUCILP2 or commercial recombinant irisin or saline. At day three, the cells were refreshed with the same media as day one. After treatments for six hours on day 3, C2C12 cells were harvested for qRT-PCR analysis.

### (6) Effects of Recombinant RUCILP2 on Immortalized Hepatic Carcinoma Cells, the HepG2 Cell Line

HepG2 cells were seeded on 12-well plates under DMEM/F-12 medium (Dulbecco's Modified Eagle Medium/Nutrient Mixture F-12, Fisher Scientific, #11524436), supplemented with 10% FBS (Fetal Bovine Serum, Fisher Scientific, #11550356) and 1% Penicillin-Streptomycin (Fisher Scientific, #11548876). Cell cultures were maintained in a humidified chamber with 5% C02 at 37° C., and culture media were changed every 2-3 days.

At 70% confluence, to induce insulin resistance, HepG2 cells were incubated with 18 mM glucosamine (GlcN) for 18 h in serum-free medium, followed by treatment with recombinant RUCILP2 or commercial recombinant irisin (Sigma, #SRP8039-10UG and Phoenix pharmaceuticals, #067-29A), or saline for 24 h. For integrin inhibitor treatment, cells were treated with 10 μM of CycloRGDyK (Selleckchem, #S7844) for 10 minutes and washed with PBS before treated with recombinant RUCILP2 or commercial recombinant irisin or saline. After treatments, HepG2 cells were harvested for qRT-PCR analysis.

### (7) Effects of Recombinant RUCILP2 on Immortalized Human Colorectal Adenocarcinoma Cells, the Caco-2 Cell Line

Commercially available Caco-2 cells (ATCC, #HTB-37) were seeded on 12-well plates under EMEM medium (Eagle's Minimum Essential Medium, ATCC, #30-2003), supplemented with 20% FBS (Fetal Bovine Serum, Fisher Scientific, #11550356) and 1% Penicillin-Streptomycin (Fisher Scientific, #11548876). Cell cultures were maintained in a humidified chamber with 5% CO₂ at 37° C., and culture media were changed every 2-3 days.

At 70% confluence, to induce the hypoxia/reoxygenation (H/R) cell culture model, Caco-2 cells were cultured in EMEM medium (glucose and FBS free) and exposed to hypoxia conditions (94% N₂, 5% C02 and 1% O₂) at 37° C. for 120 minutes. Next, cells were treated for 24 hours with indicated concentrations of recombinant RUCILP2 or commercial recombinant irisin (Sigma, #SRP8039-10UG and Phoenix pharmaceuticals, #067-29A) or PBS immediately at the beginning of reoxygenation. For integrin inhibitor treatment, cells were treated with 10 μM of CycloRGDyK (Selleckchem, #S7844) for 10 minutes and washed with PBS before treated with recombinant RUCILP2 or commercial recombinant irisin or PBS. After treatments, Caco-2 cells were harvested for qRT-PCR analysis of mRNA level of intestinal epithelial barrier-related genes.

### (8) Effects of Recombinant RUCILP2 on H9C2 Cell Line

Commercially available H9C2 cells (ATCC, #CRL-1446) were seeded on 12-well plates under DMEM/F-12 medium (Dulbecco's Modified Eagle Medium/Nutrient Mixture F-12, Fisher Scientific, #11524436), supplemented with 10% FBS (Fetal Bovine Serum, Fisher Scientific, #11550356) and 1% Penicillin-Streptomycin (Fisher Scientific, #11548876). Cell cultures were maintained in a humidified chamber with 5% CO₂ at 37° C., and culture media were changed every 2-3 days.

At 70% confluence, the cells were treated with indicated concentrations of recombinant RUCILP2, commercial recombinant irisin (Sigma, #SRP8039-10UG and Phoenix pharmaceuticals, #067-29A), and PBS for 24 h. For integrin inhibitor treatment, cells were treated with 10 μM of CycloRGDyK (Selleckchem, #S7844) for 10 minutes and washed with PBS before treated with recombinant RUCILP2, commercial recombinant irisin and PBS. After treatments, H9C2 cells were harvested for qRT-PCR analysis of mRNA level of cardiomyocyte growth and differentiation-related genes.

### (9) Effects of Recombinant RUCILP2 on Hormone Release in the Perfused Live Rat Colon

Animals: Male Wistar rats (˜250 g) were obtained from Janvier (Le Genest-Saint-Isle, France) and housed with two to four rats per cage. Rats were allowed one week of acclimatization kept on a 12:12 h light/dark cycle with ad libitum access to water and standard chow.

Ethical considerations: Studies were conducted with permission from the Danish Animal Experiments Inspectorate (2018-15-0201-01397) and the local ethical committee (EMED, P20-058) in accordance with the EU Directive 2010/63/EU and guidelines of Danish legislation governing animal experimentation (1987) and the National Institute of Health.

Isolation and perfusion of the proximal rat colon: At the day of experiment, non-fasted rats were anaesthetized with a subcutaneous injection of Hypnorm/Midazolam (0.0158 mg fentanyl citrate+0.5 mg fluanisone+0.25 mg midazolam/100 g). When lack of reflexes was established, rats were placed on a heating plate (37° C.) and the abdominal cavity opened. The colon was isolated by ligating the vascular supply to cecum, the small intestine, the spleen, the stomach, the kidneys as well as the celiac artery allowing isolation of the most proximal part of the colon to the part just proximal to the entry of inferior mesenteric artery (˜10 cm). A plastic tube was placed in the lumen of the colon, and the colon was gently flushed with isotonic saline (room temperature) to remove luminal contents. Throughout the experimental protocol, a constant luminal flow of saline was applied via a syringe pump (0.15 ml/min). A catheter was inserted into the abdominal aorta, which was ligated proximally to the superior mesenteric artery, and the intestine was vascularly perfused with heated (37° C.), oxygenated (95% O₂ and 5% CO₂) perfusion buffer at a constant flow rate of 3 ml/min using a single pass perfusion system (UP100, Hugo Sachs Harvard Apparatus, Germany). A metal catheter was inserted into vena portae to collect the venous effluent. As soon as proper flow was apparent, rats were euthanized by perforation of the diaphragm. To allow for equilibration of the system, the intestine was perfused for 25 minutes before initiation of the experimental protocol. Each protocol started with a baseline period followed by addition of test substance applied either through the luminal tube or intra-arterially through the catheter inserted into aorta. The venous effluent was collected for 1 min periods via the draining catheter using a fraction collector. Effluent samples were immediately placed on ice and stored at −20° C. until analysis. As an indicator of health of the colon, perfusion pressure was monitored throughout the experiment.

Perfusion buffer: Perfusion buffer consisted of a modified Krebs-Ringer bicarbonate buffer supplemented with 3.5 mmol/L glucose, 0.1% (w/v) bovine serum albumin (cat. No. 1.12018.0500, Merck, Denmark), 5% (w/v) dextran T-70 (to balance oncotic pressure; Pharmacosmos, Denmark), 5 mmol/L of each fumarate, pyruvate and glutamate (Sigma Aldrich, Brøndby, Denmark) and 10 μmol/L 3-isobutyl-1-methylxanthine (IBMX, cat no. 5879, Sigma Aldrich).

Hormone measurement: Peptide hormones were measured using in-house radioimmunoassays: total GLP-1 (the sum of 7-36NH₂, 9-36NH₂ and potential mid-terminal cleaved fragments) was measured using a C-terminal specific antibody targeting amidated forms of GLP-1 (code no. 89390). Total PYY (PYY1-36+PYY3-36) was measured with a porcine antiserum (cat. no T-4093; Bachem). Somatostatin was measured using a side-viewing antibody (code no. 1758-5), detecting all bioactive forms of somatostatin.

(10) Effects of Recombinant RUCILP2 Following Intraperitoneal Injection into Mice Fed with Chow

Mice (20 male wild type C57BL/6N, 8 weeks of age, Janvier) were single-housed at 23±1° C. on a 12-hour light/12-hour dark cycle with access to food and water ad libitum. After 7 days of acclimatization on a standard chow diet (Altromin 1328 diet contains 11% of fat, 24% of protein, and 65% of carbohydrates. This diet is a cereal-based (soy, wheat, corn) fixed formula which is free of alfalfa and fish/animal meal and deficient in nitrosamines) to avoid stress, the mice received daily intraperitoneal injection of 1 mg/kg recombinant RUCILP2, and saline, respectively, for 7 days. All animals were then sacrificed, and subcutaneous fat pads and liver tissue were collected. The mRNA levels of thermogenic genes including Ucp1, Elovl3, Cidea, and Prdm16 as well as lipogenic genes including Acaca and Fasn in inguinal fat were analyzed by qRT-PCR as described below.

RNA extraction and real-time PCR analysis: Total RNA was extracted from tissues using Trizol reagent (Invitrogen) according to the manufacturer's instructions, followed by concentration measurement. 1 μg RNA was transcribed to cDNA using the Reverse Transcription System (Promega). Real-time PCR was performed using the LC480 detection system (Roche Diagnostics) and SYBR Green I Supermix (Takara). Samples were run in duplicate in a single 384-well reaction plate. Data were normalized to the housekeeping Rp136, TBP or GAPDH genes and analyzed according to ΔΔCT method.

### Example 3—Whole-Body Effects of a RUMTOR_00181-Producing RT Strain and a Non-RUMTOR_00181 Producing RT Strain, Respectively on Mice Fed with Chow Diet

### Main Results

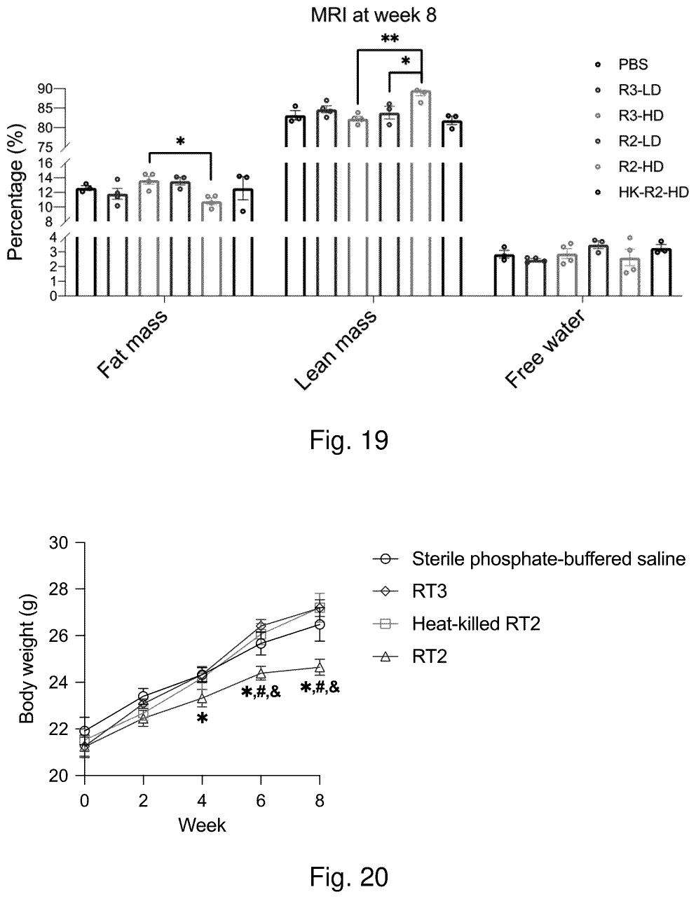

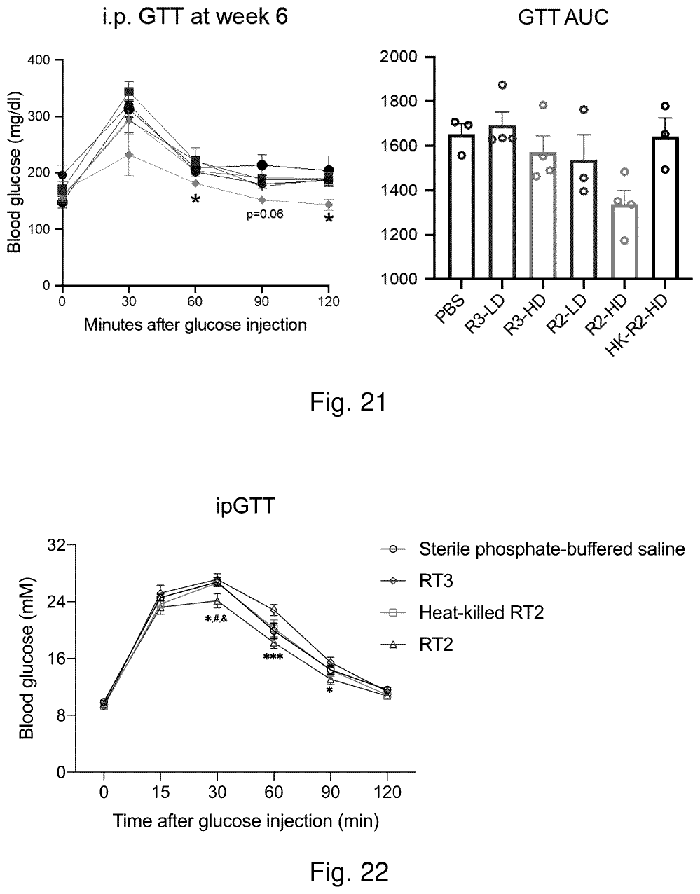

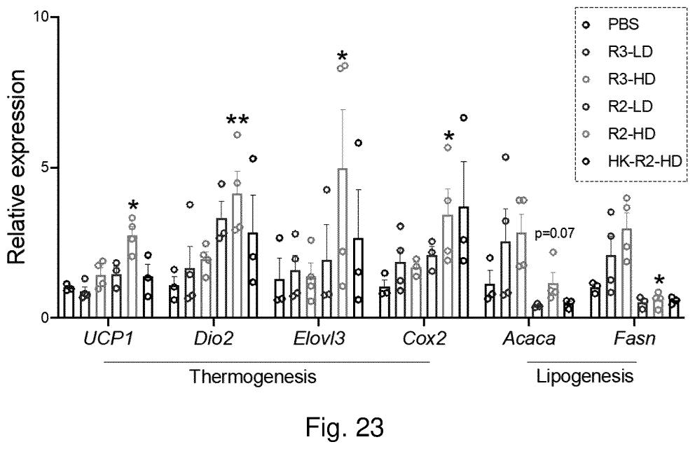

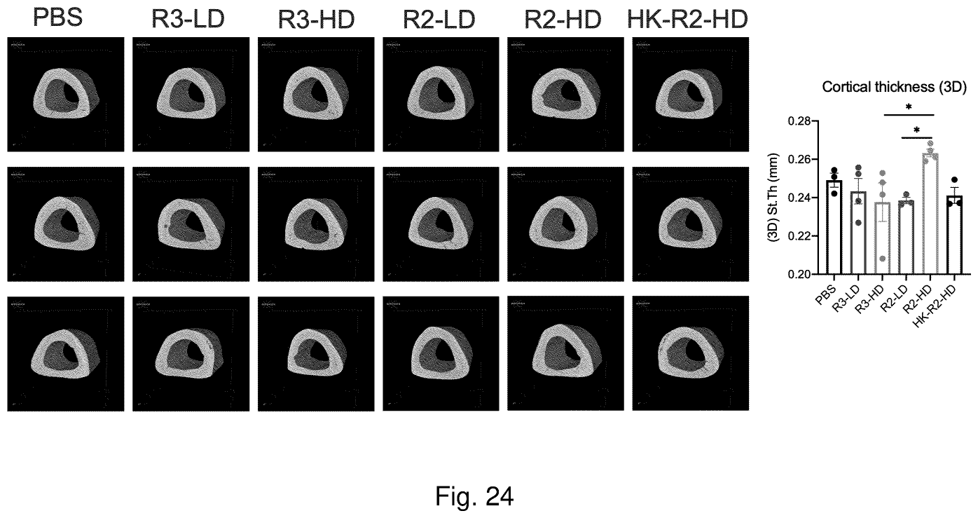

In mice fed with chow diet, effects of oral gavage twice a week for 8 weeks with either a RUMTOR_00181 producing RT strain (VPI B2-51, ATCC), or a non-RUMTOR_00181-producing RT strain (VPI 13831, ATCC) were compared. Oral gavage with a RUMTOR_00181-producing RT strain was found to reduce fat mass and increase lean mass (FIG. 19), while it had no influence on mouse body weight gain (FIG. 18). Meanwhile, the thermogenic program in inguinal fat tissue was activated, followed by reduced adipose lipogenesis (FIG. 23). The glucose tolerance was improved (FIG. 21), and in addition, cortical bone density increased (FIG. 24). In mice fed with high-fat diet it was found that the live RUMTOR_00181-producing Ruminococcus torques strain—ATCC 27756—(RT2) reduced body weight gain during an 8-week intervention (FIG. 20). In addition, an intraperitoneally injected glucose tolerance test showed that the RUMTOR_00181-producing strain—ATCC 27756—(RT2)—improved glucose tolerance in vivo in mice fed with high-fat diet (FIG. 22).

### Materials and Methods

Intervention Study in Mice with a RUMTOR_00181-Producing RT Strain and a Non-RUMTOR_00181 Producing RT Strain, Respectively

For the intervention study of mice fed with chow diet, eight-week-old male C57BL/6N mice (specific-pathogen free grade, Janvier) were single-housed with free access to chow diet containing 11% of fat, 24% of protein, and 65% of carbohydrates (Altromin 1328 diet), and water under a strict 12 h light cycle. They were then divided into six groups gavaged with sterile PBS, live RT-ATCC 35915 (5×10⁷ colony forming units (CFUs)/100 μl in sterile PBS), live RT-ATCC 35915 (5×10⁸ CFUs/100 μl in sterile PBS), live RT-ATCC 27756 (5×10⁷ CFUs/100 μl in sterile PBS), live RT-ATCC 27756 (5×10⁸ CFUs/100 μl in sterile PBS), and heat-killed RT-ATCC 27756 (5×10⁸ CFUs/100 μl in sterile PBS at 70° C. for 30 min), respectively, twice a week for 8 weeks. Body weights were measured before every gavage. For the intervention study of mice fed with high-fat diet, eight-week-old male C57BL/6N mice (specific-pathogen free grade, Janvier) were group-housed with free access to high-fat diet containing 45 kcal % fat, 20 kcal % protein, and 35 kcal % carbohydrate (Research Diet, D12451 i), and water under a strict 12 h light cycle. They were then divided into four groups gavaged with sterile PBS, live RT-ATCC 35915 (5×10⁹ colony forming units (CFUs)/100 μl in sterile PBS), live RT-ATCC 27756 (5×10⁹ CFUs/100 μl in sterile PBS), and heat-killed RT-ATCC 27756 (5×10⁹ CFUs/100 μl in sterile PBS at 70° C. for 30 min), respectively, twice a week for 8 weeks. Body weights were measured every second week. The stool samples were collected before gavage and at the end of the experiment, and immediately stored at −80° C. before further analysis. Body fat mass and body lean mass were assessed by a whole-body composition analyzer (EchoMRI). The acclimatized mice were allocated to groups based on their body weights to ensure equal starting points. At the end of the study, mice were anesthetized and blood from orbital plexus was collected in tubes containing ethylenediamine tetraacetic acid (EDTA). Blood samples were centrifuged for 6 min at 6,000 rpm, 4° C. Plasma samples were isolated and stored at −80° C. for subsequent biochemical testing. Tissue samples (liver, brown adipose tissue, subcutaneous adipose tissue, mesenteric adipose tissue, jejunum, ileum and proximal colon) were dissected, weighed, and stored at −80° C. for further analysis. A part of the adipose tissues, and one of the tibia bones were fixed in 4% paraformaldehyde in PBS for histological analysis or micro-CT scanning.

For glucose tolerance testing after 6 weeks of bacterial treatment, all mice were returned to standard drinking water, fasted for 4 hours, weighed, and then given a bolus of glucose (2 g glucose/kg body weight) by intraperitoneal injection. Blood samples were taken 0, 15, 30, 60, and 120 minutes from the tail vein for the measurement of blood glucose using a glucose meter (LifeScan).

### Example 4—Proteomics Assay for Measurement of RUCILP2 in Human Plasma

### Main Results

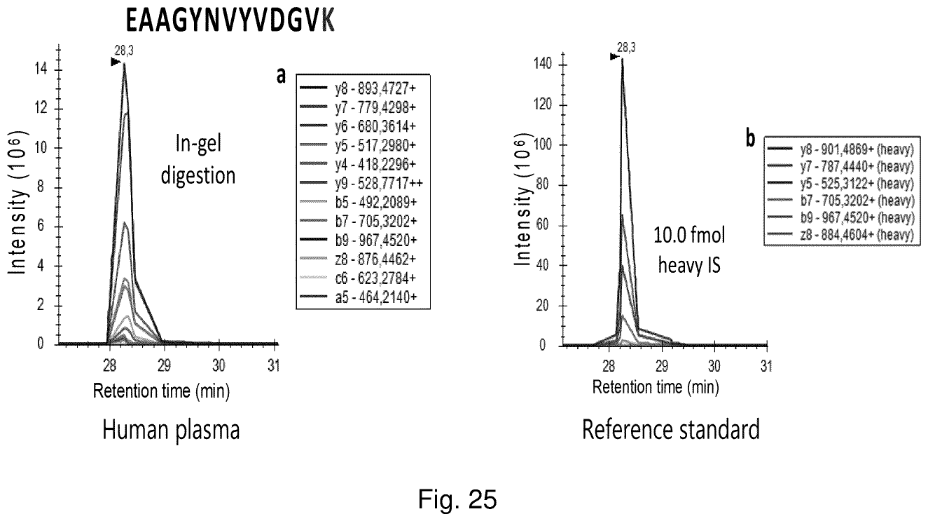

The developed targeted proteomics assay indicated that RUCILP2 circulates in human plasma at an inter-individual concentration interval of 10-100 μg/ml measured from 6 individuals (FIG. 25).

### Materials & Methods

### Protocol for Proteomics Sample Preparation

(1) Human plasma specimens (1 ml) were depleted of albumin and IgG using the ProteoExtract kit (Millipore, 122642).
   1.1 Dilute the desired amount of sample with 10× Binding Buffer and water
   1.2 Mount a column or a column assembly for syringe tip filter usage
   1.3 Fill a syringe with. 6-10 ml of 1× Binding Buffer for column equilibration. Remove any trapped air from the syringe before connecting with the column
   1.4 By application of gentle pressure allow a volume of 2 ml 1× Binding Buffer per column to pass through the resin bed. Discard the flow through
   1.5 Fill a new syringe with the diluted sample. By application of gentle pressure allow diluted sample to pass through the column. Collect the column(s) flow-through. Note: Count to five in between two consecutive droplets to allow for sufficient contact time between the sample and the resin
   1.6 Connect the syringe of step 3 filled with 1× Binding Buffer to the column(s). By application of gentle pressure allow a volume of 2 ml buffer per column to pass the resin bed. Collect the column(s) flow-through as the wash fraction. Combine wash fraction with the previously collected flow-through as depleted samples
(2) Depleted samples were subsequently concentrated using 3 kiloDalton (kDa) molecular weight cut-off spin-filter columns (Millipore, UFC900324)
(3) Deglycosylation of plasma was performed using Protein Deglycosylation Mix II kit (New England Biolabs) under denaturing reaction conditions

| | |
|---|---|
| 3.1 | Dissolve 100 μg of glycoprotein into 40 μl water |
| 3.2 | Add 5 μl of Deglycosylation Mix Buffer 2 |
| 3.3 | Incubate at 75° C. for 10 minutes, cool down |
| 3.4 | Add 5 μl Protein Deglycosylation Mix II, mix gently |
| 3.5 | Incubate reaction at 25° C. (room temperature) for 30 minutes |
| 3.6 | Transfer reaction to 37° C., incubate for 1 hour |

### In-Gel Digestion Step

(1) Deglycosylated plasma samples (300 μg) were reduced with 10 mM Dithiothreitol (DTT, Sigma) and alkylated with 50 mM iodoacetamide prior to being resolved by SDS-PAGE using 4%-12% NuPAGE Bis-Tris precast gels (Biorad 4-12% Criterion™ XT Bis-Tris Protein Gel, 18 well, 30 μl, #3450124)
(2) Gels were Coomassie stained, and fragments were excised from the 10-15 kDa region
(3) Gel pieces were destained and dehydrated with 100% acetonitrile, vacuumed dried
(4) The dried residues were resuspended with 200 μl of Tris-Urea Buffer (8 M urea (sigma) in 0.1 M Tris-HCl, pH 8.5) and added on the centrifugal spin filter unit (0.5 ml, molecular weight cut off at 10 kDa) and centrifuge at 10000 g until less than 10 μl the sample remains in the filter. This usually requires centrifugation time of 10-15 min. This applies to all further centrifugation steps
(5) Add 200 μl of UA to the filter unit and repeat the centrifugation
(6) Discard the flow-through form the collection tube
(7) Add 100 μl of DB (0.05 M Tris-HCl, pH 8.5) to the filter unit and centrifuge at 10000 g for 10 min. Repeat this step twice
(8) Add 100 μl of 50 mM ammonium bicarbonate (Sigma-Aldrich), and 500 ng sequencing-grade trypsin (Promega) for an overnight incubation at 37° C. directly onto the filter membrane
(9) Add 20 μl of 1× internal standard mix (=2 picomolar) directly onto filter membrane
(10) After 12 h, centrifuge the filter units at 10000 g until the solution completely passed the filter membrane (about 5 min)
(11) Add 100 μl of digestion buffer (0.05 M Tris-HCl, pH 8.5) and centrifuge the filter units at 10000 g until all liquid has passed through. Sample volume should now be approximately 200 μl
(12) Peptides are de-salted using Millipore C18 ZipTips (Sigma)
(13) Peptides were eluted with 5 μl of 70% acetonitrile and 1% formic acid, then dried using a speedvac.

### The Step of Liquid Chromatography-Mass Spectrometer (LC-MS) Detection

Mass spectrometry data were collected using a Q Exactive or LTQ Orbitrap Elite mass spectrometer (Thermo Fisher Scientific) coupled with a Famos Autosampler (LC Packings) and an Accela 600 liquid chromatography (LC pump (Thermo Fisher Scientific). Peptides were separated onto a 100-μm inner diameter microcapillary column packed with ˜0.5 cm of Magic C4 resin (5 μm, Michrom Bioresources) followed by ˜20 cm of Accucore C18 resin (1.6 μm, Thermo Fisher Scientific). For each analysis, ˜4 μl was loaded onto the column. Peptides were separated using a 50-min gradient of 8%-30% acetonitrile in 0.125% formic acid with a flow rate of ˜250 nl/min.

### The Step of Parallel Reaction Monitoring (PRM) of Targeted Mass Spectrometry

PRM analyses were performed with a Q Exactive mass spectrometer (Thermo Fisher Scientific) using the following parameters: a full MS scan from 400 to 700 Thomson (Th) at an orbitrap resolution of 70,000 (at m/z 200), automatic gain control (AGC) target 5×10⁶, and a 500 ms (millisecond) maximum injection time. Full MS scans were followed by 25-50 PRM scans at 35,000 resolution (at m/z 200) (AGC target 5×10⁶, 500 ms maximum injection time) as triggered by a scheduled inclusion list. The PRM method employed an isolation of target ions by a 2 Th (Thomson) isolation window, fragmented with normalized collision energy (NCE) of 25. MS/MS scans were acquired with a starting mass range of 100 m/z and acquired as a profile spectrum data type. Precursor and fragment ions were quantified using Skyline version 3.1.

### Data-Dependent Acquisition Step

For data-dependent acquisitions using Q Exactive, the scan sequence began with an Orbitrap MS1 spectrum with the following parameters: resolution 70,000, scan range 400-1,400 Th, automatic gain control (AGC) target of 5×10⁶, maximum injection time of 250 ms, and centroid spectrum data type. We selected the top 20 precursors for MS² analysis, which consisted of high-energy collision dissociation (HCD) with the following parameters: resolution 17,500, AGC 1×10⁵, maximum injection time 60 ms, isolation window 2 Th, NCE 25, and acquired as a centroid spectrum data type. The underfill ratio was set at 9%, which corresponds to a 1.5×10⁵ intensity threshold. In addition, unassigned and singly charged species were excluded from MS² analysis and dynamic exclusion was set to automatic.

For data-dependent acquisitions using a Linear Trap Quadropole (LTQ) Orbitrap Elite, the first analyzer, MS1, survey scan was performed in the orbitrap in the range of 300-1,500 Th at a resolution of 3×10⁴. This was followed by the selection of the ten most intense ions (TOP10) for collision-induced dissociation (CID)-MS² fragmentation using a precursor isolation width window of 2 Th. The AGC settings were 3×10⁶ and 2.5×10⁵ ions for survey and MS² scans, respectively. Ions were selected for MS² when their intensity reached a threshold of 500 counts and an isotopic envelope was assigned.

Maximum ion accumulation times were set to 1,000 ms for survey MS scans and to 250 ms for MS² scans. Singly charged ion species and ions for which a charge state could not be determined were not subjected to MS². Ions within a 10 parts per million (ppm) m/zwindow around ions selected for MS² were excluded from further selection for fragmentation for 120 s.

### Peptide and Protein Identification Step

Following mass spectrometry data acquisition, Thermo Fisher RAW files were converted into an eXtensible Markup Language (mzXML) format and processed using a suite of software tools developed in-house for analysis of proteomics datasets. All precursors selected for MS/MS fragmentation were confirmed using algorithms to detect and correct errors in monoisotopic peak assignment and refine precursor ion mass measurements. All MS/MS spectra were then exported as individual .DTA files and searched using the Sequest algorithm. These spectra were searched against a database containing sequences of all human proteins reported by Uniprot in both forward and reversed orientations. Common contaminating protein sequences (e.g., human keratins, porcine trypsin) were included as well. The following parameters were selected to identify peptides from unenriched peptide samples: 25 ppm precursor mass tolerance, 0.02 Da product ion mass tolerance, no enzyme digestion, up to two tryptic missed cleavages, Variable modifications: oxidation of methionine (+15.994915) and deamidation of aspargine (0.984016), fixed modifications: carbamidomethylation of cysteine (+57.021464). The AScore algorithm was implemented to quantify the confidence with which each deamidation modification could be assigned to a particular residue in each peptide. Peptides with AScores above 13 were considered to be localized to a particular residue (p<0.05).

### Example 5—Identification and In Vitro Functional Characterization of 21-AABP2

### Main Results

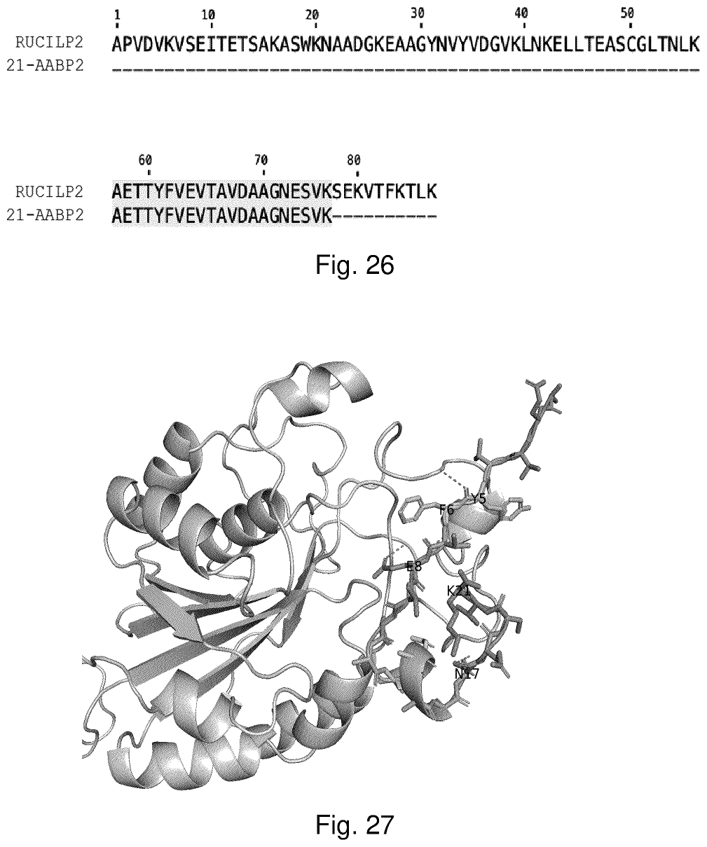

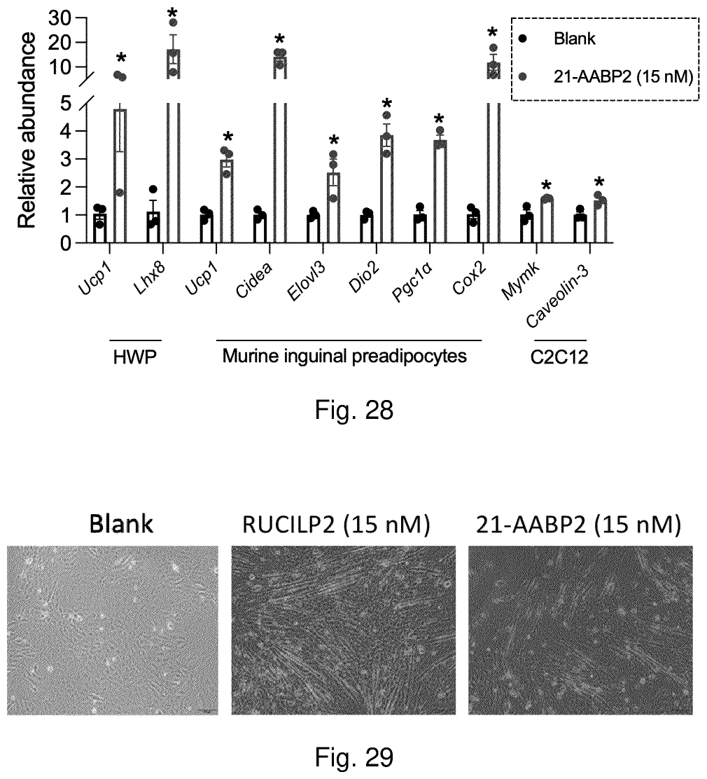

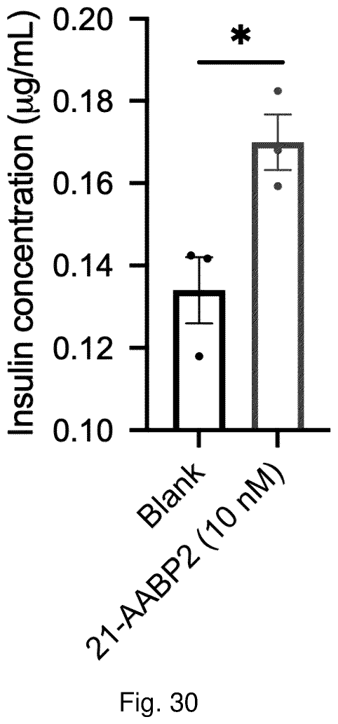

One of the trypsin cleaved fragment of RUCILP2, a 21 amino acid bacterial peptide 2 (termed 21-AABP2, FIG. 26) was predicted as the only fragmental peptide to have higher hydrophobic score than that of RUCILP2, meaning 21-AABP2 may exhibit higher protein structural or functional stability than its precursor, RUCILP2. Moreover, 21-AABP2 was predicted to bind to the RUCILP2 receptor (αV/β5 integrin receptor, FIG. 27). The peptide 21-AABP2 was demonstrated as an inducer of key genes regulating thermogenesis in human visceral white pre-adipocytes and in mouse inguinal pre-adipocytes (FIG. 28). In murine myoblasts, 21-AABP2 facilitated myogenesis and myotube formation (FIG. 29). In addition, 21-AABP2 stimulated insulin release from a rat insulinoma cell line (FIG. 30).

### Materials & Methods

### Prediction of the Hydrophobic Score for RUCILP2 and its Trypsin-Cleaved Fragmental Peptides

Theoretical protein cleavage of RUCILP2 after trypsin digestion was processed by PeptideMass (https://web.expasv.ora/peptide mass/). Peptide hydrophobicity was predicted by PEPTIDE 2.0 using the default settings (https://www.peptide2.com/N peptide hydrophobicity hydrophilicity.ph).

### Predicted Interaction Between 21-AABP2 and αV/β5 Integrin Receptor

The 3D protein structure of 21-AABP2 was predicted by PEP-FOLD. RUCILP2 or 21-AABP2 was docked to αV/β5 receptor using the ZDOCK server according to the provider's guidelines. Complex 3D structures were visualized by PyMOL. The docking model of 21-AABP2 and integrin receptor was performed by ZDOCK server and the complex with the highest docking score was selected as the best binding model.

Synthesis of Recombinant 21-AABP2 in Escherichia coli

Target DNA sequence of the 21 amino acid 21-AABP2 was optimized and synthesized. The synthesized sequence was cloned into vector pET-30a (+) with 6-His-tag for protein expression in E. coli strain BL21 star (DE3) that was transformed with recombinant plasmid. A single colony was inoculated into Terrific Broth (TB) medium containing related antibiotic; the culture was incubated at 37° C. with 200 rpm and then induced with isopropyl β-D-1-thiogalactopyranoside (IPTG). SDS-PAGE was used to monitor the expression. Recombinant BL21 star (DE3) stored in glycerol was inoculated into TB medium containing related antibiotic and cultured at 37° C. When the OD 600 reached about 1.2, cell culture was induced with IPTG at 37° C./4h. Cells were harvested by centrifugation. Cell pellets were resuspended with lysis buffer followed by sonication. The supernatant after centrifugation was kept for future purification. Target protein was obtained by one-step purification using Ni column. Target protein was kept in 50 mM Tris-HCl, 150 mM NaCl, 10% Glycerol, pH 8.0 followed by sterilized by 0.22 μm filter before stored in aliquots. The concentration was determined by a bicinchoninic acid (BCA) TM protein assay with bovine serum albumin (BSA) as standard. The protein purity and molecular weight were determined by standard SDS-PAGE along with Western blot confirmation. The protein was diluted in sterilized phosphate-buffered saline (PBS) to use in cell culture experiments.

### Cellular Metabolic Effects of 21-AABP2

### (1) Recombinant 21-AABP2 Promotes Expression of Key Genes of Thermogenesis and Browning in Human Visceral White Pre-Adipocytes

White pre-adipocytes from human visceral fat (C-12732, PromoCell) were cultured until 80% confluent and switched into differentiation media (with 0.3 ml/ml of fetal calf serum (FCS), 8ug/ml of d-biotin, 0.5ug/ml of insulin, 400 ng/ml of dexamethasone) in the presence of 15 nM of 21-AABP2. The differentiation process to mature adipocytes was completed after 14 days. Cells were harvested after 14 days of differentiation and the thermogenesis-related genes including Ucp1 and Lhx8were quantified by q-PCR.

### (2) Recombinant 21-AABP2 Induces Expression of Key Genes Regulating Thermogenesis in Mouse Inguinal Pre-Adipocytes

Inguinal fat tissues from 6-week-old wild-type C57BL/6J female mice were dissected and washed with PBS, minced and digested for 1 hour at 37° C. in PBS containing 10 mM CaCl₂, 2.4 U/ml dispase II (Roche) and 10 mg/ml collagenase D (Roche). After adding warm DMEM/F12 (1:1) with 10% FCS, digested tissue was filtered through a 70 mm cell strainer and centrifuged at 600×g for 10 minutes. Pellet was resuspended by 40 ml DMEM/F12 (1:1) with 10% FCS and filtered through a 40 mm cell strainer followed by centrifugation at 600×g for 10 minutes. Pelleted inguinal stromal vascular cells were grown to confluence and split onto 12-well plates. The cells were induced to differentiate by treatment with 1 mM rosiglitazone, 5 mM dexamethasone, 0.5 mM isobutyl methyl xanthine for 2 days. Next, cells were maintained in 1 mM rosiglitazone for 4 days with medium change every other day. The cells were treated with 15 nM of 21-AABP2 every other day during 6 days of differentiation. Cells were harvested after 6 days of differentiation and thermogenesis genes including Ucp1, Cidea, Elovl3, Dio2, and Pgc1a were quantified by q-PCR.

### (3) Recombinant 21-AABP2 Stimulates Myogenesis and Myotube Formation in Murine C2C12 Myoblasts

C2C12 myoblasts were cultured until 80% confluent and switched into differentiation media (with 2% horse serum). The treatment with 21-AABP2 started from the second day of differentiation. Representative images of the formed myotubes at 24h of differentiation in the presence of PBS (blank) or 21-AABP2 (15 nM) were captured. Cells were harvested after 4 days of differentiation and expression of myogenesis genes including Mymk and Caveolin-3 were quantified by q-PCR.

(4) Recombinant 21-AABP2 Stimulates Insulin Release from Immortalized Rat Insulinoma Cells, INS-1 Cells

INS-1 cells were grown in in RPMI 1640 medium (11875093, ThermoFisher Scientific) until a confluency of 70% was achieved and switched to RPMI 1640 medium supplied with 15 nM of 21-AABP2 and incubated for 12 hours. The insulin concentration in supernatant of cell culture medium was measured by MSD rat/mouse insulin ELISA kit (Merck Millipore).

### Example 6—Physiological Effects of Oral Gavage with a Live RUMTOR_00181 Producing RT2 Strain on Mice Fed Either Chow or a High-Fat Diet

### Introduction

Through a comprehensive bioinformatics search for the presence of human hormone-like sequences in publicly available genomes of prokaryotes, we identified two significant homologies, both of which are from a CoDing Sequence (CDS) annotated as RUMTOR_00181 (Uniprot: A5KIY5) in the genome of Ruminococcus torques ATCC 27756. The genomic information of R. torques ATCC 27756 has been deposited at NCBI as the reference genome and used as the type strain for bacterial species R. torques.

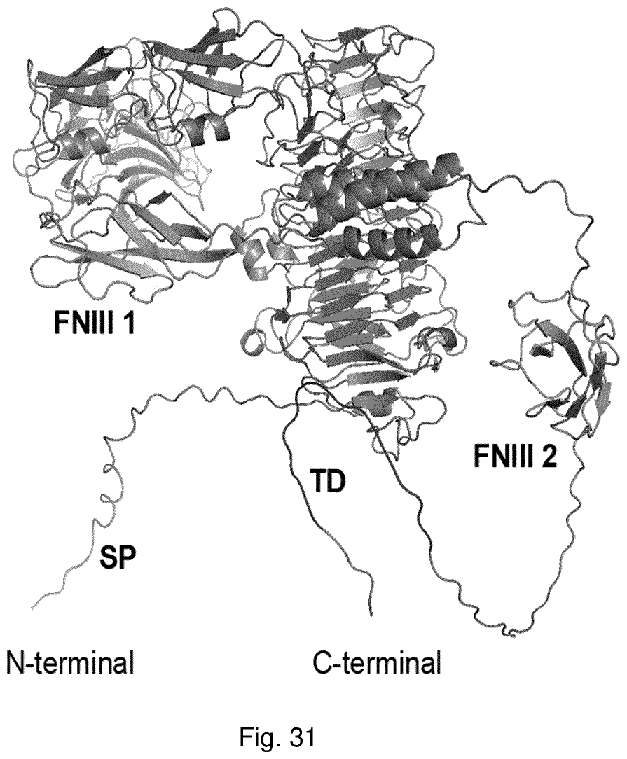

The 3D structure of RUMTOR_00181 protein predicted by AlphaFold2²² demonstrates a signal peptide at N-terminal, two fibronectin type III (FNIII) domains and one hydrophobic domain that is likely to be membrane inserted, followed by a 7-amino acid C-terminal domain (FIG. 31).

R. torques species has a 26 reported strains. Within the genomes of three additional strains from R. torques we discovered the presence of predicted proteins with high homology with the RUMTOR_00181 protein. Among the genes encoding RUMTOR_00181 and RUMTOR_00181-like proteins in the four R. torques strains, 1,260 of 1,271 amino acid residues are conserved. Pairwise comparisons show RUMTOR_00181-like sequences in R. torques AM22-16, R. torques aa_0143, and R. torques 2789STDY5834841. These are 99.5% (1,265 of 1,271), 99.5% (1,265 of 1,271), and 99.4% (1,263 of 1,271), respectively, identical to RUMTOR_00181 in R. torques ATCC 27756.

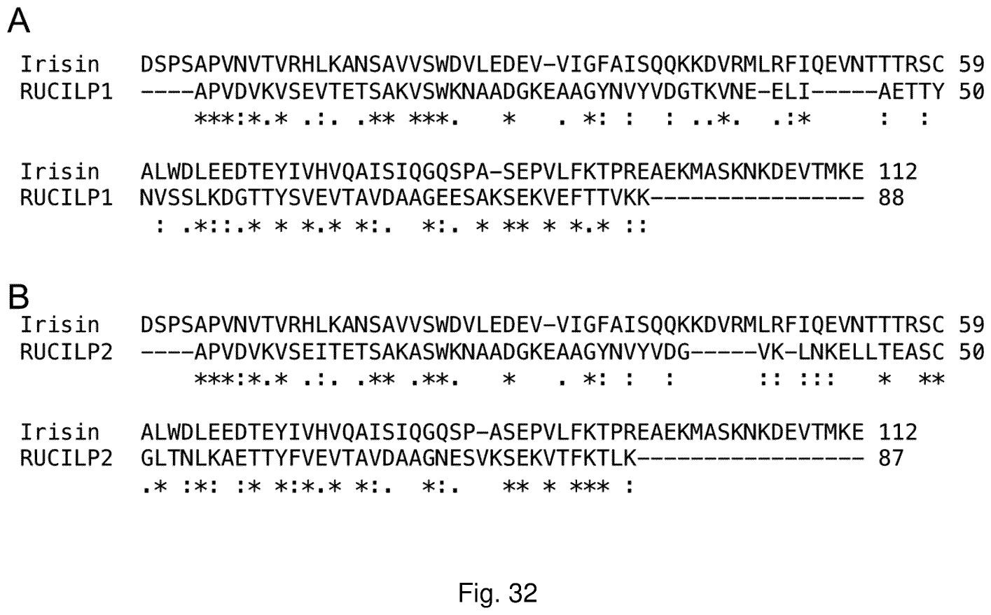

Intriguingly, we found that the two FNIII domains in RUMTOR_00181 show respective homological identities of 30.7% and 34.5% to a recently discovered myokine, irisin (FIG. 32). Therefore, the two FNIII containing domains were named as Ruminococcus torques irisin-like peptide 1 (RUCILP1 for short) and RUCILP2, with 88 and 87 amino acid residues, respectively.

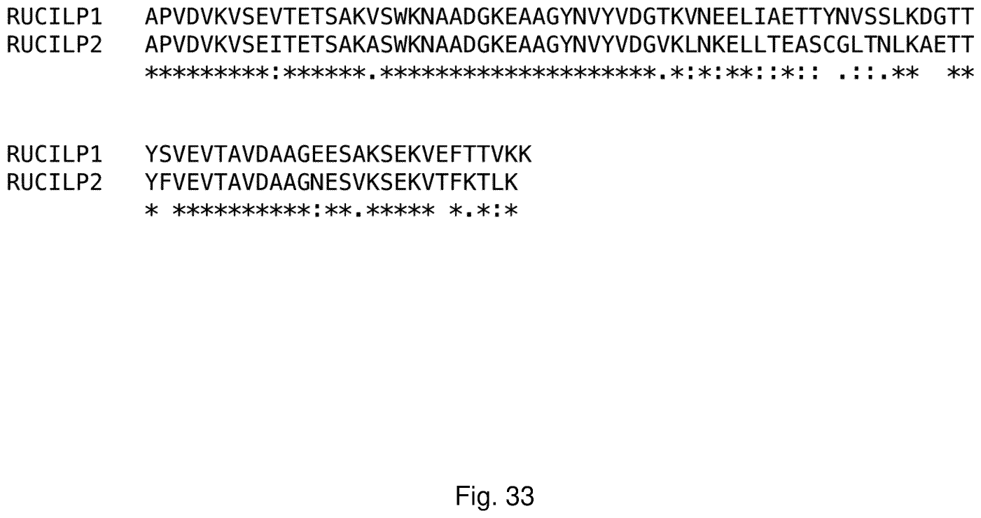

By applying the EMBOSS Needle Pairwise Sequence Alignment Program (https://www.ebi.ac.uk/Tools/psa/emboss needle/), paired alignment for RUCILP1 vs RUCILP2 demonstrates RUCILP1 has a 73.9% (65/88) identity to RUCILP2 (FIG. 33).

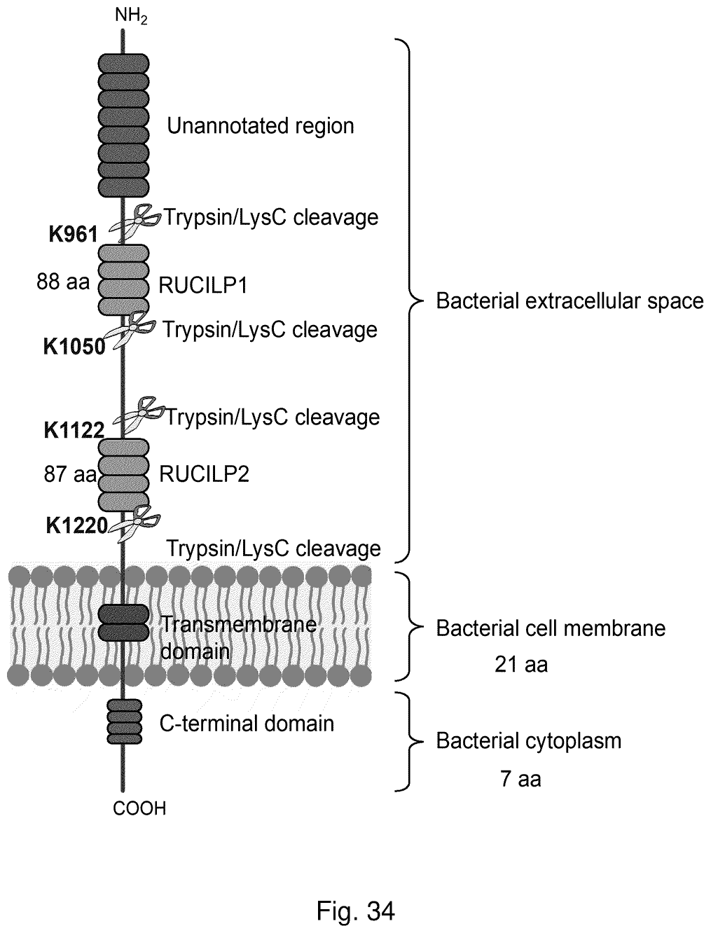

RUCILP1 and RUCILP2 are considered to be released by RUMTOR_00181 producing strains through an extracellular trypsin/LysC-dependent proteolytic cleavages at K961, K1050, K1122, and K1220. respectively (FIG. 34).

Given that RUCILP2 has relatively higher identity to irisin, we then recombinantly synthesized RUCILP2 from Escherichia coli and explored its physiological effects. In cellular and animal experiments, we have shown that recombinant RUCILP2 and irisin in equimolar concentration (cellular studies) or dose (in vivo studies) have similar effects on both expression of key genes of thermogenesis and browning in murine and human visceral adipocytes. RUCILP2 enhances leptin expression in fat cells and inhibits expression of genes regulating lipogenesis in adipocytes and liver cells. In addition, in liver cells, the hormone inhibits the expression of genes regulating gluconeogenesis. RUCILP2 stimulates insulin biosynthesis and in live rat colon perfusion experiments, RUCILP2 exhibits strong stimulatory effects on luminal release of GLP-1, GLP-2, PYY and somatostatin.

In the following, we summarized the main findings of two interventions with oral supplementation of a live RUMTOR_00181 producing strain in mice with C57BL/6N background (specific pathogen-free grade) aged 8 weeks at study start. One intervention was in chow fed mice. The other was in high-fat-fed mice. Each intervention was run for eight weeks.

### Main Results

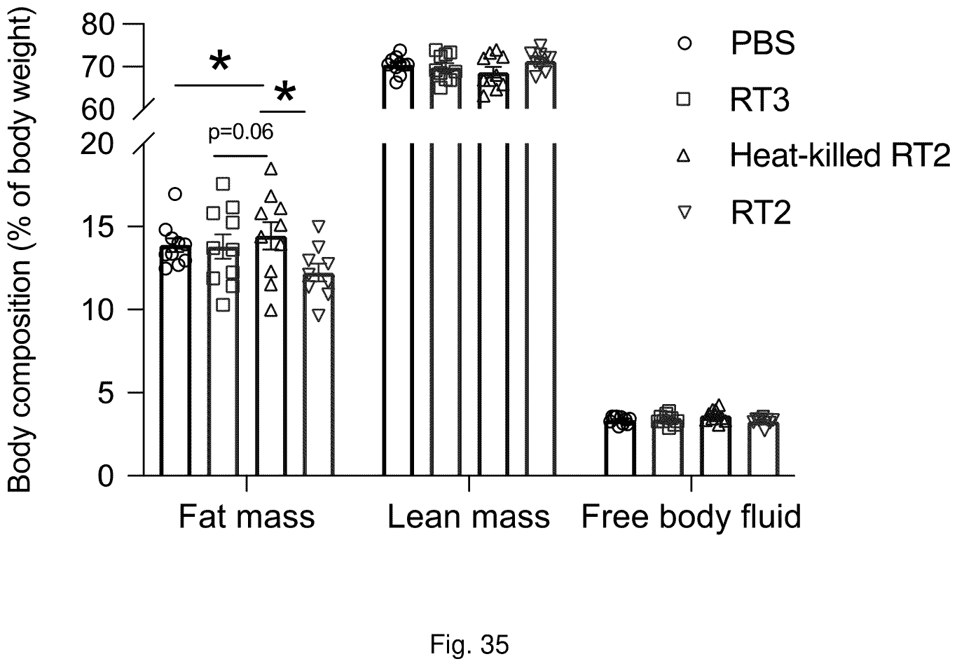

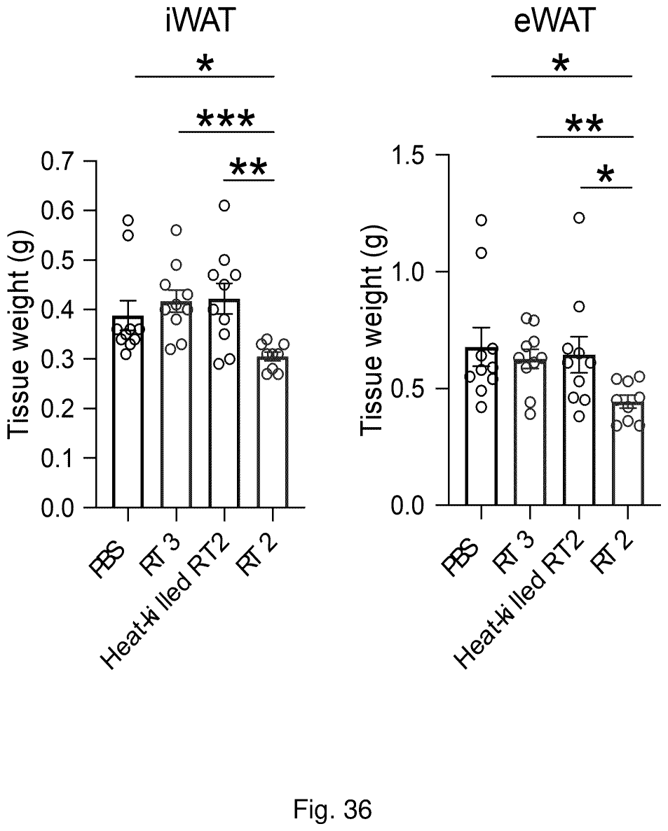

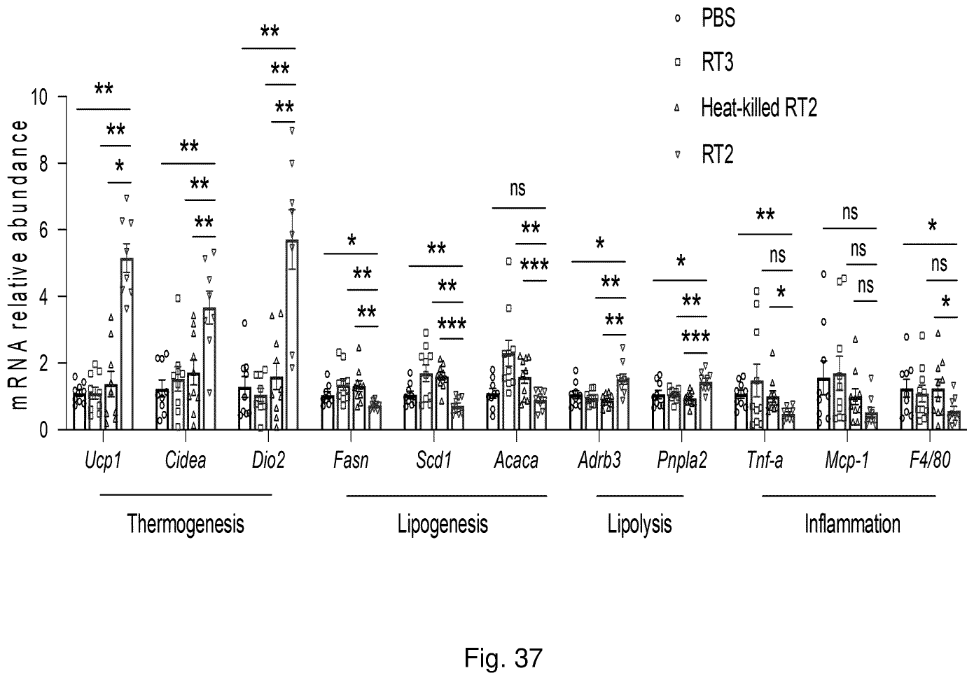

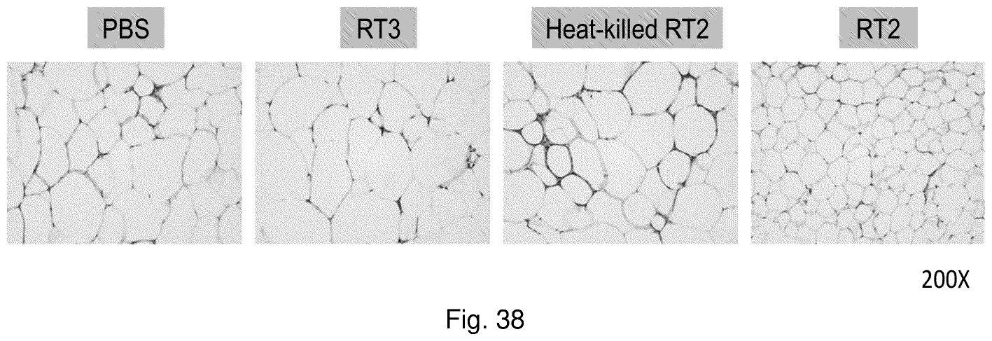

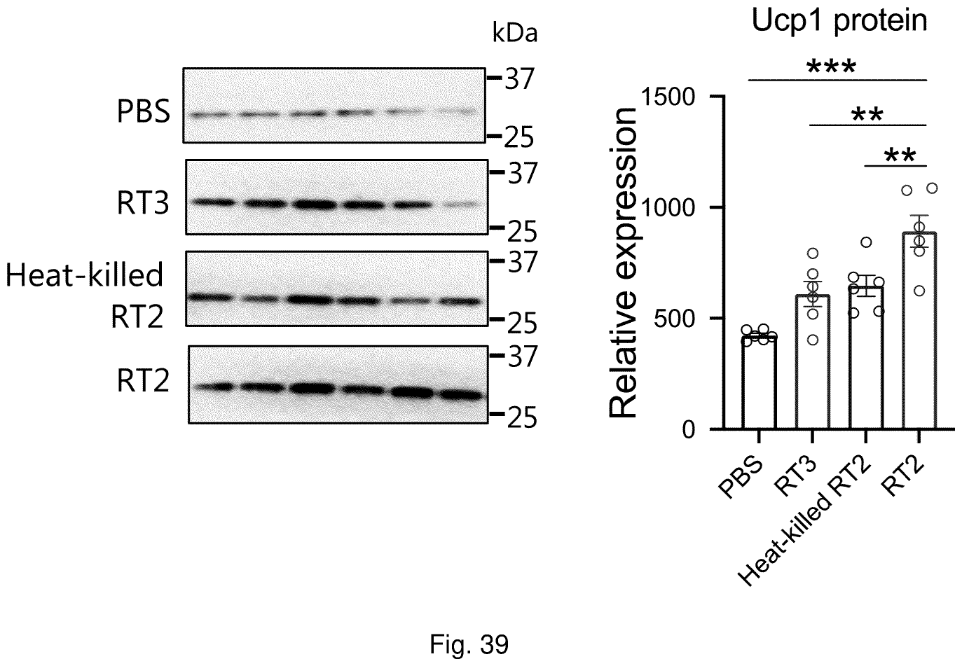

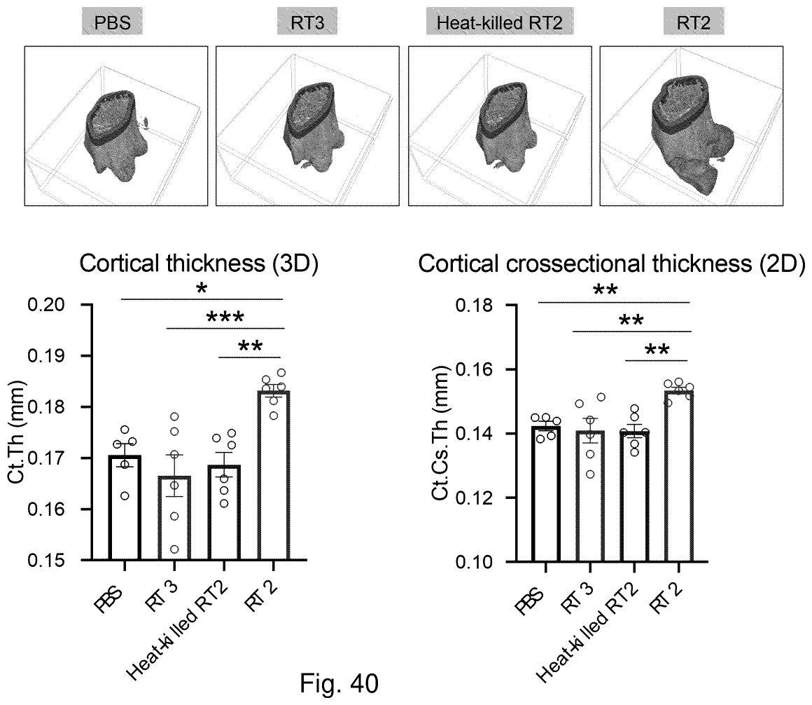

In C57BL/6N mice fed a chow diet, effects of oral gavage twice a week for eight weeks with either a live RUMTOR_00181 producing R. torques ATCC 27756 (RT2) strain, or a live non-RUMTOR_00181-producing R. torques ATCC 35915 (RT3) strain were compared. Oral gavage with RT2 strain reduced body fat mass and increased lean body mass (FIG. 19), while it had no influence on mouse body weight gain over study period (FIG. 18). Meanwhile, gene expression analyses showed that the thermogenic program in inguinal fat tissue was activated with simultaneous reduction of adipose tissue lipogenesis (FIG. 23). The glucose tolerance was improved (FIG. 21), and in addition, cortical thickness of proximal tibia increased (FIG. 24). In mice fed a high-fat diet (HFD) we found that the live RT2 strain reduced body weight gain during an eight-week intervention (FIG. 19). Meanwhile, magnetic resonance imaging (MRI) scanning of the body compositions showed the RT2 supplementation significantly reduced mouse fat mass and increased lean mass (FIG. 35). As indicated by lower weight of inguinal and epididymal white adipose tissue mass in RT2-supplemented mice, it is concluded that RT2 colonization reduced adiposity gain over time in mice fed HFD (FIG. 36). In addition, an intraperitoneal glucose tolerance test showed that the RT2 strain improved glucose tolerance in HFD fed mice (FIG. 22). Gene analysis in the inguinal fat demonstrated enhanced expression of mRNA encoding thermogenic markers, including Ucp1, Cidea, and Dio2, in RT2-gavaged mice, whereas the genes involved in adipose lipogenesis, including Fasn, Scd1, and Acaca, were attenuated. Consistently, we found activated lipolysis in subcutaneous white adipose tissue cells from the RT2-supplemented mice. In addition, makers of white adipose tissue inflammation, including Tnf-α, Mcp-1, and F4/80, of HFD-fed mice were dampened in response to RT2 intervention (FIG. 37). Histological analysis of inguinal fat revealed a substantially smaller adipocyte cell size in HFD mice gavaged with live RT2 than in HFD mice gavaged with phosphate-buffered saline (PBS) or heat-killed RT2 or live RT3 strain (FIG. 38). Apart from the reduction in the adipocyte size of white adipose tissue, the expression of UCP1 at protein level was enhanced in the inguinal fat tissue (FIG. 39). Notably, we detected a remarkably higher distal femur bone mass in HFD fed mice following the intervention by RT2 (FIG. 40).

### Materials & Methods

Culture of Ruminococcus torques Strains

The RUMTOR_00181-positive R. torques ATCC 27756 (RT2) and RUMTOR_00181-negative R. torques ATCC 35915 (RT3) strains were purchased from ATCC Bacteriology Collection and cultured under anaerobic conditions (95% N₂, 5% H₂) in ATCC medium #1589 containing modified chopped meat with 1% glucose (Anaerobe Systems #AS-813) overnight.

For the oral gavage in mice, cultures of both strains were centrifuged at 6,000 g for 10 min, washed with phosphate-buffered saline (PBS) twice and concentrated in anaerobic PBS with 20% (vol/vol) glycerol anaerobically to 5×10¹⁰colony-forming units per ml (CFUs/ml).

The bacterial counting was determined using tryptic soy medium with 5% defibrinated sheep blood and 1.5% agar (ATCC Medium #260). Additionally, concentrated RT2 strains at 5×10¹⁰ CFUs/ml were autoclaved at 121° C. for 15 min. A viability confirmation was performed by culture showing that heat-killed RT2 did not grow at all, while live RT2 strain grew well.

Prior to oral administration to mice, the stock bacterial solutions were thawed and diluted to 5×10′ CFUs/ml, 5×10⁸ CFUs/ml, and 5×10⁹ CFUs/ml for corresponding experiments.

### Protocols for Interventions in Mice

All mice were purchased from Janvier Labs (Le Genest-Saint-Isle, France) with a C57BL/6N background (specific pathogen-free grade). Animal experiments were performed with approved protocols from the Danish Animal Experiments Inspectorate (license number: 2018-15-0201-01491), and the University of Copenhagen (project number: P20-392). The mice were housed in following conditions: all mice were housed in an enriched environment at 23±1° C. on a 12-hour light/1 2-hour dark cycle with ad libitum access to food and tap water, unless otherwise stated. Male mice were used for all in vivo studies. Mice were allowed to acclimatize to the above environment under standard chow diet for 1 week prior to any experiment performed. Acclimation was carried out in open cages in a constant climate chamber (Memmert, HPP750). Mice were group-housed unless relevant phenotyping strategies (indirect calorimetry) required single housing.

For the intervention study of mice fed a chow diet, eight-week-old male C57BL/6N mice (specific-pathogen free grade, Janvier) were single-housed with free access to chow diet (see description below) and water under a strict 12 h light cycle. They were then divided into six groups gavaged with sterile PBS, low dose live RT3 (RT3-LD, 5×10⁷ colony forming units (CFUs)/100 μl in sterile PBS), high dose live RT3 (RT3-HD, 5×10⁸ CFUs/100 μl in sterile PBS), low dose live RT2 (RT2-LD, 5×10⁷ CFUs/100 μl in sterile PBS), high dose live RT2 (RT2-HD, 5×10⁸ CFUs/100 μl in sterile PBS), and heat-killed RT2 (HK-RT2, 5×10⁸ CFUs/100 μl in sterile PBS at 121° C. for 15 min), respectively, twice a week for 8 weeks. Body weights were measured before every gavage.

For the intervention study of mice fed a high-fat diet, eight-week-old male C57BL/6N mice (specific-pathogen free grade, Janvier) were group-housed with free access to high-fat diet (see below) and water under a strict 12 h light cycle. They were then divided into four groups gavaged with sterile PBS, live RT3 (5×10⁹ colony forming units (CFUs)/100 μl in sterile PBS), live RT2 (5×10⁹ CFUs/100 μl in sterile PBS), and heat-killed RT2 (5×10⁹ CFUs/100 μl in sterile PBS at 121° C. for 15 min), respectively, twice a week for 8 weeks. Body weights were measured every second week. Stool samples were collected before gavage and at the end of the experiment, and immediately stored at −80° C. before further analysis. Body fat mass and body lean mass were assessed by a whole-body composition analyzer (EchoMRI). The acclimatized mice were allocated to groups based on their body weights to ensure equal starting points. At the end of the study, mice were anesthetized and blood from orbital plexus was collected in tubes containing ethylenediamine tetraacetic acid (EDTA). Blood samples were centrifuged for 6 min at 6,000 rpm, 4° C. Plasma samples were isolated and stored at −80° C. for subsequent biochemical analyses.

### Diets

The standard chow diet (Altromin 1328 diet) contains 11% of fat, 24% of protein, and 65% of carbohydrates. This diet is a cereal-based (soy, wheat, corn) fixed formula which is free of alfalfa and fish/animal meal and deficient in nitrosamines. High-fat diet (Research Diet, D12451 i) is formulated by 45 kcal % fat (lard and soybean oil), 20 kcal % protein (Casein), and 35 kcal % carbohydrate (sucrose, lodex 10, and starch).

### Tissue Sampling

Tissue samples (liver, interscapular brown adipose tissue, inguinal white adipose tissue, epidydimal white adipose tissue, jejunum, ileum and proximal colon) were dissected, weighed, and stored at −80° C. for later analysis. A part of the adipose tissues, and one of the tibia and femur bones were fixed in 4% paraformaldehyde in PBS for histological analysis or micro-CT scanning.

### Glucose Tolerance Test

For glucose tolerance testing after 6 weeks of bacterial treatment, all mice were returned to standard drinking water, fasted for 4 hours, weighed, and then given a bolus of glucose (2 g glucose/kg body weight) by intraperitoneal injection. Blood samples were taken 0, 15, 30, 60, and 120 minutes from the tail vein for the measurement of blood glucose using a glucose meter (LifeScan).

### Histological Analysis

Mice inguinal white adipose tissue (iWAT) depots were fixed in 4% paraformaldehyde/1×PBS overnight at 4° C., followed by immersed in 100% of ethanol for 24 h prior to paraffin embedding. For the determination of adipocyte size, adipose tissue paraffin sections were stained with hematoxylin and eosin (H&E staining). Images were obtained under bright-field microscopy. A representative image for each group was shown in our study and the adipocyte cell diameter was measured from the H&E-stained slides using open-source ImageJ software.

### RNA Extraction and Quantification

For the total RNA extraction of tissue samples isolated from mice, after adding with one sterile and stainless steel bead (Qiagen, #69989) and 500 I of QIAzol Lysis Reagent, each piece of frozen tissue sample (weighted at around 50 mg) was homogenized. After homogenizing, samples were centrifuged at 12,000 g, 15 minutes, 4° C., with supernatant collected. RNA extraction was subsequently performed according to the instructions provided by manufacturer of RNeasy mini kit (Qiagen, #74106), followed by measurements of RNA purities and concentrations by NanoDrop™ 2000/2000c Spectrophotometers (Thermo Fisher, #ND2000CLAPTOP). A total of 1 μg of RNA was used for reverse transcription to cDNA using High-Capacity RNA-to-cDNA™ Kit (Fisher Scientific, #10704217) following protocol and heating program. cDNA samples were subjected to real-time PCR using LightCycler® 480 System (Roche Diagnostics) after premixing with Precision®PLUS Master Mix (Primer Design, #PPLUS-machine type). For each indicated gene, samples were run in white 384-well plates and ΔΔCt method was used for quantifying RNA expression levels.

### Western Blot Analysis

The total protein from iWAT was extracted using radioimmuno-precipitation assay (RIPA) lysis buffer (Sigma-Aldrich) premixed with a cocktail containing protease and phosphatase inhibitors (Sigma-Aldrich). Prior to the SDS-PAGE on a 4-20% polyacrylamide gel, extracted proteins were measured with Pierce BCA Protein Assay kit (Thermo Fisher Scientific), diluted with loading dye and heated at 96° C. for 10 minutes. The proteins were then subjected to immunoblot assay with UCP1 (ab10983, Abcam), and β-actin antibodies (ab115777, Abcam) was used as internal controls. The LAS 4000 (Life Science) system was used to visualize the membranes according to the providers' guides.

### Micro-CT Analysis of Mouse Proximal Tibia and Distal Femur

We used high-resolution desktop microcomputed tomography imaging (Skyscan 1172, Bruker) for scanning the proximal tibia from mice fed a chow diet and distal femur from HFD-fed mice. Morphometric analysis on cortical microstructures of proximal tibia and distal femur was performed as follows: X-ray voltage of 50 kV, X-ray current of 200 μA, fiter of 0.5 mm aluminium, image pixel size of 4-5 m, camera resolution of 1,280 pixel field width, tomographic rotation of 180°/360°, rotation step of 0.3-0.5°, frame averaging of 1-2 with a scan duration of 30-50 minutes.

The metaphyseal-diaphyseal cortex was selected with reference to the growth plate. A crossectional slice was selected as a growth plate reference slice as follows: moving slice-by-slice toward the growth plate from the metaphysis/diaphysis, a point is reached where a clear “bridge” of low-density cartilage (chondrocyte seam) becomes established from one corner of the crossection to another. This bridge is established by the disappearance of the last band of fine primary spongiosal bone interrupting the chondrocyte seam. This landmark allows a reference level to be defined for the growth plate. Cortical volumes of interest were then defined relative to this reference level. The cortical region commenced about 2.15 mm (500 image slices) from the growth plate level in the direction of the metaphysis and extended from this position for a further 0.43 mm (100 image slices). 3D and 2D morphometric parameters were calculated for the cortical selected region of interests (ROIs). 3D parameters were based on analysis of a Marching Cubes type model with a rendered surface. Calculation of 2D areas and perimeters were based on the Pratt algorithm. Structure thickness in 3D was calculated using the local thickness or “sphere-fitting” method, and structure model index (an indicator of the relative prevalence of plates and rods) was derived according to the method of Hildebrand and Ruegsegger. Degree of anisotropy was calculated by the mean intercept method. Rendered 3D models were constructed for 3D viewing of cortical analysed regions. Model construction was by the “Double time cubes” method, a modification of the Marching cubes method. Cortical morphometric parameters measured by micro-CT include 3D cortical thickness (Ct.Th, mm), 2D cortical crossectional thickness (Ct.Cs.Th mm), cortical periosteal perimeter (Ct.Pe.Pm, mm), cortical endosteal perimeter (Ct.En.Pm, mm), cortical crossectional area (Ct.Ar, mm²), Polar moment inertia (MMI(p), mm⁴), Eccentricity (Ecc), and cortical porosity (Ct.Po, %).

All measurements were done blinded for the examiner.

### Example 7—Identification of Binding Epitopes in RUCILP1 and RUCILP2 to Integrin αV/β5 Receptor Using SPOT Peptide Microarray Assay

As shown, RUCILP1 and RUCILP2 may exert their multiple metabolism-beneficial effects via binding to the integrin αV/β5 receptor. This Example aimed at identifying the binding epitopes of both proteins to the receptor by performing an unbiased and semi-quantitative SPOT peptide microarray (μSPOT) assay.

### Materials & Methods

### Synthesis of 15-Mer Peptides of Both Proteins for μSPOT Assay

μSPOT peptide arrays²⁵ (CelluSpots, Intavis AG, Cologne, Germany) were synthesized using a RePepSL synthesizer (Intavis AG) on acid labile, amino functionalized, cellulose membrane discs (Intavis AG) containing 9-fluorenylmethy-loxycarbonyl-β-alanine (Fmoc-β-Ala) linkers (minimum loading 1.0 μmol/cm). Synthesis was initiated by Fmoc deprotection using 20% piperidine in N-methylpyrrolidone (NMP) (1×2 and 1 ×4 μL, 3 and 5 min, respectively) followed by washing with dimethylforma-mide (DMF, 7×100 μL per disc) and ethanol (EtOH, 3×300 μL per disc). The loading of the discs were reduced to 50%, by using a mixture of Fmoc-Gly-OH and Boc-Gly-OH (0.25 M: 0.25 M in NMP). All couplings were achieved using 1.2 μL of coupling solution consisting of preactivated amino acids (AAs, 0.5 M) with ethyl 2-cyano-2-(hydroxyimino)acetate oxyma (1.5 M) and N,N′-diisopropylcarbodiimide (DIC, 1.1 M) in NMP (2:1:1, AA:oxyma:DIC). The couplings were carried out 6 times (5, 10, 20, 30, 30, and 30 min, respectively), and subsequently, the membrane was capped twice with capping mixture (5% acidic anhydride in NMP), followed by washes with DMF (7×100 μL per disc). After chain elongation, final Fmoc deprotection was performed with 20% piperidine in NMP (3×4 μL, 5 min each), followed by 6× washes with DMF, subsequent N-terminal acetylation with capping mixture (3×4 μL, 5 min each) and final washes with DMF (7×100 μL per disc) and EtOH (7×200 μL per disc). Dried cellulose membrane discs were transferred to 96 deep-well blocks and were treated with the side-chain deprotection solution consisting of 80% TFA, 12% DCM, 5% H2O, and 3% TIPS (150 μL per well) for 1.5 h at room temperature (rt). Afterward, the deprotection solution was removed, and the discs were solubilized overnight at rt using a solvation mixture containing 88.5% TFA, 4% trifluoromethansufonic acid (TFMSA), 5% H2O, and 2.5% TIPS (250 μL per well). The resulting peptide-cellulose conjugates were precipitated with ice-cold ether (700 μL per well) and spun down at 1000 rpm for 90 min, followed by an additional wash of the formed pellet with ice-cold ether. The resulting pellets were redissolved in DMSO (250 μL per well) to give final stocks, which were transferred to a 384-well plate and printed (in duplicates) on white coated CelluSpots blank slides (76×26 mm, Intavis AG) using a SlideSpotter robot (Intavis AG).

### Visualization and Analysis of μSPOT Assay

After the peptide array slides were rinsed with 100 mM phosphate buffered saline (PBS) (pH 7.4), the arrays were blocked with 3% bovine serum albumin (BSA) in PBS for >2 h at rt. Subsequently, the arrays were incubated with His-tagged Integrin receptor (2.5 nM) in blocking for 1 h at rt. After 5×1 min washes with blocking buffer the slides were probed with HRP-conjugated 6x-His tag antibody (1:10000, ab184607, abcam) for 0.5 h at rt. Finally, the slides were washed 3×1 min with PBS, 2×1 min with PBST, 2×1 min PBS at rt. The washed arrays visualized using SuperSignal West Femto Maximum Sensitivity Substrate (Thermo Scientific) with Fusion FX SPectra multimodal imaging platform (Vilber). The resulting blots were analyzed using the Array Analyze Software (Active Motif), which defines the error range of each data set by comparing the intensities of each peptide duplicate on the analyzed array.

### Results

We first generated a library of 15-mer peptides covering the entire sequence of both proteins to map systematically their interactions with integrin αV/β5 receptor. This resulted in 74 and 73 peptides for RUCILP1 (SEQ ID NO:s 22-95) and RUCILP2 (SEQ ID NO:s 96-168), respectively, that were chemically synthesized on acid labile, amino functionalized, cellulose membrane discs.

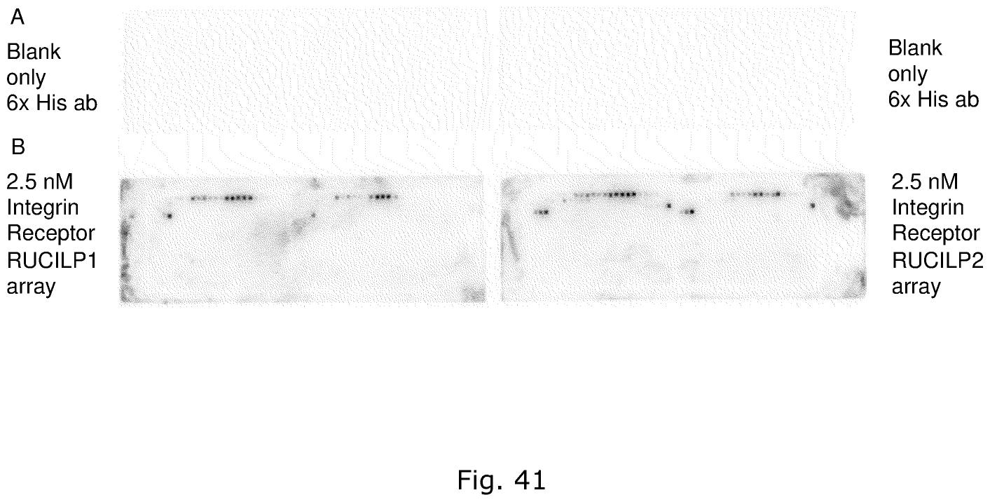

Prior to the initial screening, we confirmed that the secondary antibody produces no non-specific binding (‘background binding’) to the coated microscopic slide (FIG. 41A). The relative binding affinity of the resulting peptides was then evaluated in a semi-quantitative manner by screening the μSPOT peptide array with recombinantly expressed His-tag integrin αV/β5 (2.5 nM), followed by incubation with a horse radish peroxidase (HRP) conjugated 6x-His antibody (FIG. 41B).

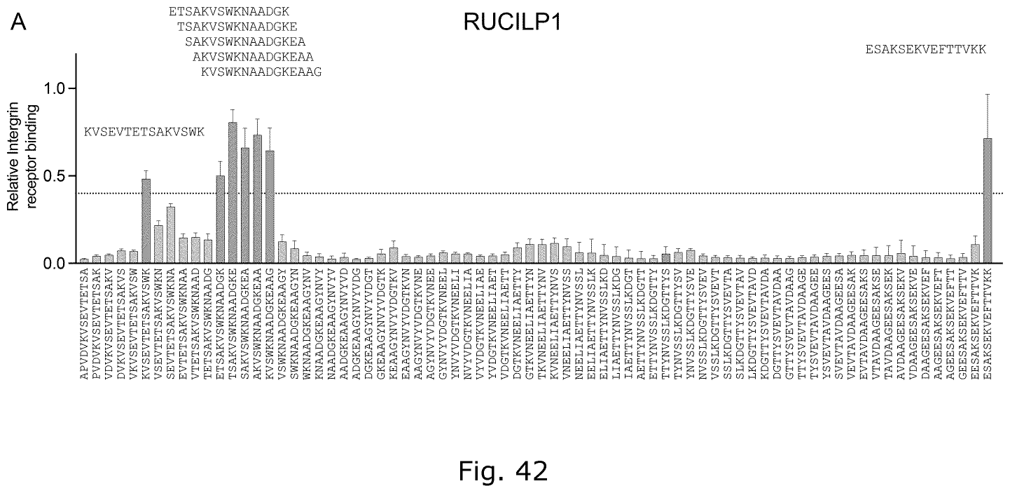

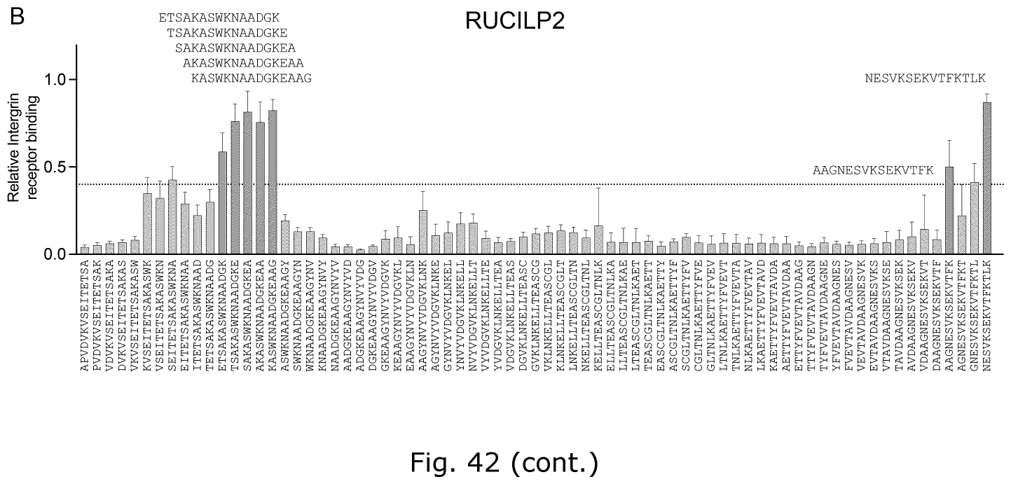

After visualization of the peptide array, we identified two binding epitopes containing residues ¹²ETSAKVSWKNAADGKEAAG³⁰ (SEQ ID NO: 169; RUCILP1) and ¹²ETSAKASWKNAADGKEAAG³⁰ (SEQ ID NO: 183; RUCILP2), respectively (FIGS. 42A and 42B). In addition, the very C-terminal sequence of both proteins ⁷⁴ESAKSEKVEFTTVKK⁸⁸ (SEQ ID NO: 95; RUCILP1) and ⁷³NESVKSEKVTFKTLK⁸⁷ (SEQ ID NO: 168; RUCILP2) showed relatively high affinity towards the integrin receptor (FIGS. 42A and 42B).

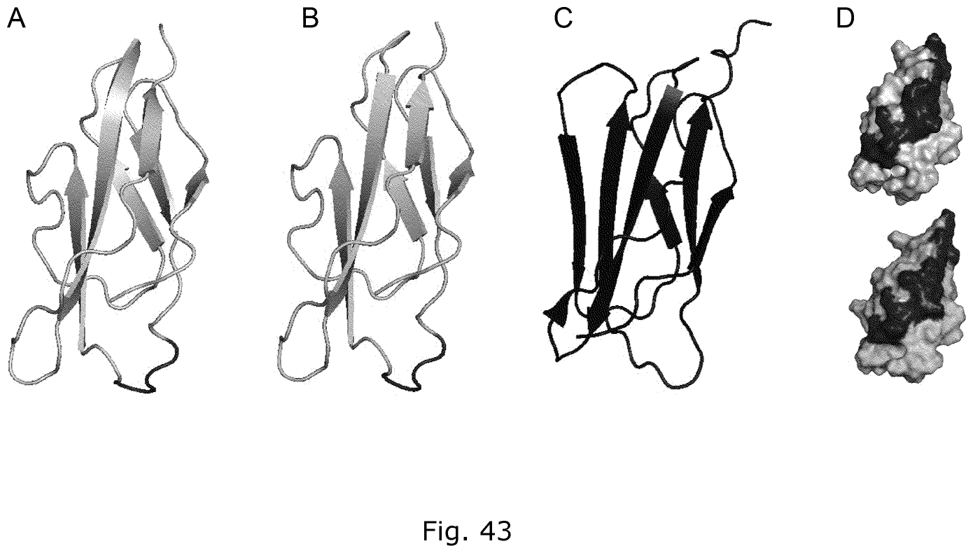

The identified binding region at the N terminus for both proteins were consistent with our AlphaFold modeling, where we predicted that loops lie at the same region (FIGS. 43A and 43B).

The flexible loops are segments of the protein that bring secondary structure elements together and are typically found on the surface of a protein. They are largely responsible for interaction with other proteins, such as a putative receptor.

In fact, the identified binding region in the two proteins correspond to the loop in irisin that potentially interacts with the same integrin receptor²⁶ (FIG. 43C). Our AlphaFold models also predicted the C-tail of both proteins as a flexible element that locates on the surface of the protein (FIG. 43D). Therefore, it is reasonable to find additional binding hits towards the receptor at the C terminus of both proteins.

### Conclusion

The SPOT peptide microarray assay makes it possible to experimentally validate in silico prediction of binding of a protein to its receptor. In our preliminary in silico prediction, we found another potential loop located at the residues ⁶⁹DAA⁷¹ in both proteins. However, the outcome of the present experiments does not support the predicted binding in RUCILP1, while in RUCILP2 we found a 15-mer peptide ⁷⁰AAGNESVKSEKVTFK⁸⁴ (SEQ ID NO: 165) including two alanine residues of the predicted loop, that showed significant binding to αV/β5 integrin receptor.

### Example 8—Head-to-Head Bioactivity Comparisons of RUCILP1 and RUCILP2 In Vitro and In Vivo

RUCILP2 has been shown to have multiple favorable effects on metabolism in both cellular and rodent studies. Here we performed a head-to-head comparison of the effects of RUCILP1 and RUCILP2 both in vitro and in vivo.

### Materials & Methods

### Cell Culture Experiments

3T3-L1 cells (a murine fibroblast cell line) were split onto 12-well plates and grown to confluence before inducing differentiation by treatment with 86 nM insulin, 0.1 μM dexamethasone, and 250 μM methyl isobutylxanthine. Two days after induction, cells were switched to an inducing medium in the presence of recombinant RUCILPs, or commercial recombinant irisin (Sigma, #SRP8039-10UG and Phoenix pharmaceuticals, #067-29A), or phosphate buffered saline (PBS) for two days. Subsequently, cells were maintained in 86 nM insulin in the presence of indicated concentrations of recombinant RUCILPs, 21-AABP1, commercial recombinant irisin, or PBS for four days with medium change every other day for 6 days. Cells were then harvested for q-PCR analysis as described in standard protocol for gene expression analysis.

MLO-Y4 cells (murine osteocyte-like cell line) were seeded on type I collagen-coated 6-well plates under Minimum Essential Medium (α-MEM from Fisher Scientific, #15430584), supplemented with 2.5% Fetal Bovine Serum (FBS from Fisher Scientific, #11550356), 2.5% calf serum (Hyclone, SH30072.03), and 1% Penicillin-Streptomycin (Fisher Scientific, #11548876). Cell cultures were maintained in a humidified chamber with 5% CO₂ at 37° C., and culture media were changed every 2-3 days. At 60% confluence, medium was switched to FreeStyle293 Expression medium after washing with warm PBS. After 4 hours of incubation, the cells were treated with recombinant RUCILPs or commercial recombinant irisin (Sigma, #SRP8039-10UG and Phoenix pharmaceuticals, #067-29A) or PBS for 24 h. After treatments, MLO-Y4 cells were harvested for qRT-PCR analysis of the mRNA level of sclerostin as described in the protocol for standard gene expression analysis.

C2C12 cells (murine myoblast cell line) were seeded on 12-well plates under DMEM/F-12 medium (Dulbecco's Modified Eagle Medium/Nutrient Mixture F-12, Fisher Scientific, #11524436), supplemented with 10% FBS (Fetal Bovine Serum, Fisher Scientific, #11550356) and 1% Penicillin-Streptomycin (Fisher Scientific, #11548876). Cell cultures were maintained in a humidified chamber with 5% CO₂ at 37° C., and culture media were changed every 2-3 days. At approximately 80% confluence, 10% fetal bovine serum was replaced with 2% horse serum to induce C2C12 myoblast cells to differentiate into myotubes. Twenty-four hours later (day 1 of the differentiation), the cells were treated with recombinant RUCILPs or commercial recombinant irisin (Sigma, #SRP8039-10UG and Phoenix pharmaceuticals, #067-29A) or PBS. At day three, the cells were refreshed with the same media as day one. After treatments for six hours on day 3, the cells were harvested for qRT-PCR analysis of mRNA levels of indicated genes.

### Experiments in Mice

All mice were purchased from Janvier Labs (Le Genest-Saint-Isle, France) with a C57BL/6N background (specific pathogen-free grade). Animal experiments were performed with approved protocols from the Danish Animal Experiments Inspectorate (license number: 2018-15-0201-01491), and the University of Copenhagen (project number: P20-392). The mice were housed in following conditions: all mice were housed in an enriched environment at 23±1° C. on a 12-hour light/1 2-hour dark cycle with ad libitum access to food and tap water, unless otherwise stated. Male mice were used for all these in vivo studies. Mice were allowed to acclimatize to the above environment under standard chow diet for 1 week prior to any experiment performed. Acclimation was carried out in open cages in a constant climate chamber (Memmert, HPP750). Mice were group-housed unless relevant phenotyping strategies (indirect calorimetry) required single housing.

For the RUCILPs intervention study, male mice (n=6 per group, 8 weeks of age) fed with standard chow diet were treated daily with an in intraperitoneal injection of 1 mg/kg recombinant RUCILP1, RUCILP2, or saline, respectively, for a week. All animals were then sacrificed and subcutaneous fat pads and liver were collected. The mRNA levels of indicated genes in subcutaneous fat and liver were analyzed by qRT-PCR as described in standard protocol for gene expression analysis.

### Gene Expression Analysis in Cells and Mouse Tissues

Total RNA from cells or tissues was extracted according to the instructions provided by manufacturer of RNeasy mini kit (Qiagen) using QIAzol lysis reagent (Qiagen), followed by measurements of RNA purities and concentrations by NanoDrop 2000 spectrophotometer (Thermo Scientific). A total of 1 μg of RNA was used for the reverse transcription to cDNA using the iScript™ Select cDNA Synthesis Kit (Bio-Rad Laboratories). The samples were subjected to real-time PCR using the LC480 system (Roche Diagnostics) after premixing with Precision®PLUS Master Mix (Primer Design). For each indicated gene, samples were run in duplicate in a 384-well plate and ΔΔCt method was used for quantifying the expressions after normalization to the housekeeping Rpl36 or GAPDH gene.

### Results

Head-to-Head Comparison of the Bioactivity of RUCILP1 and RUCILP2 when Measured In Vitro

Firstly, we tested the dose-response effects of RUCILPs on gene expression in mouse fibroblasts (3T3-L1), mouse osteoblasts (MLO-Y4), and mouse myoblasts (C2C12). To estimate effect on adipocyte browning, we measured expression of Ucp1, Prdm16, Pgc1a, Dio2 and Cox2, and expression of AdipoQ was measured as a marker of white adipocytes. As a marker of bone formation, we measured expression of Sclerostin; and markers of myotube formation included expression of Heyl, Sox3, and Stat3.

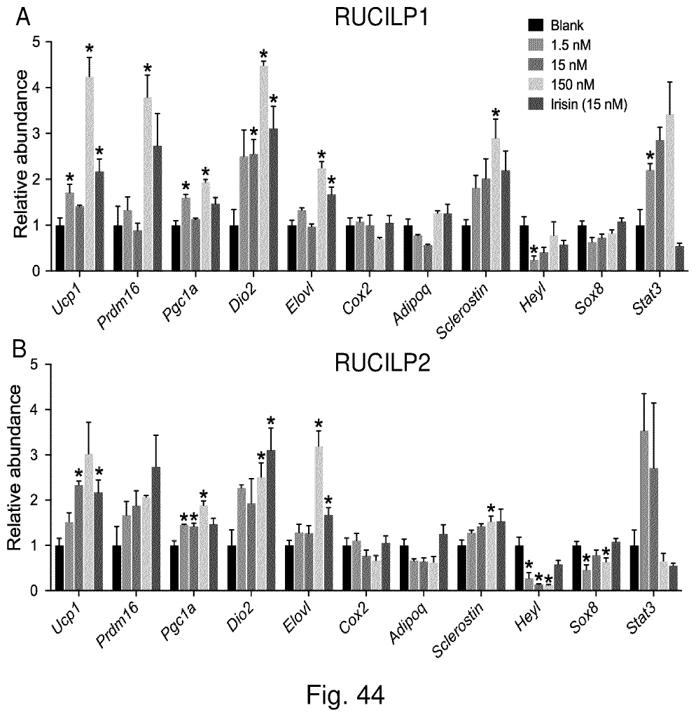

As shown in FIG. 44, in mouse fibroblasts, we observed that both RUCILPs up-regulate markers of adipocyte browning in a dose-dependent manner, while down-regulating Adiponectin expression. In osteocytes, RUCILP1 and RUCILP2 increase sclerostin expression levels at a dose of 150 nM. In myoblasts, expression of Heyl show a more clear dose-dependence response upon RUCILP2 stimulation than after RUCILP1 exposure, while the other two myotube formation markers (Sox8 and Stat3) respond in less dose-dependent fashion.

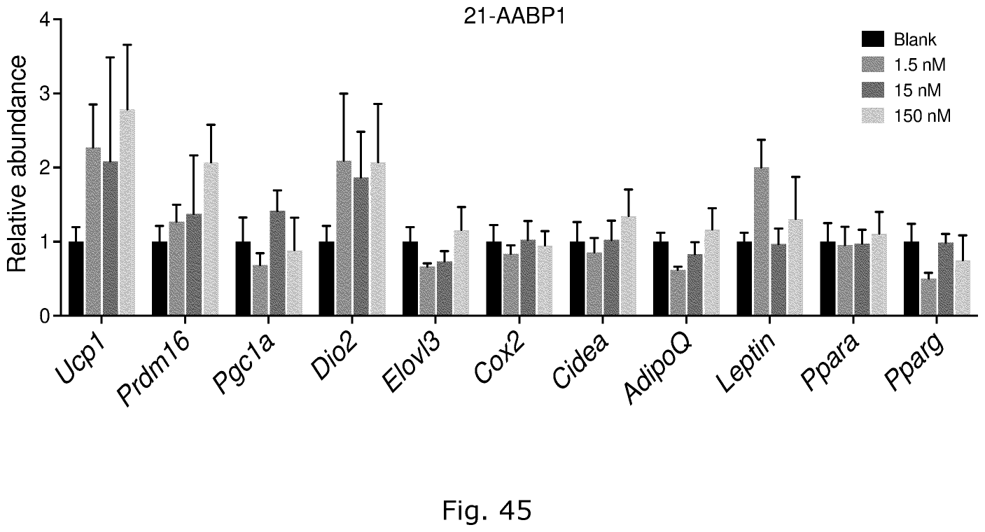

In addition to RUCILP1 and RUCILP2, we also tested 21-AABP1, the 21-amino acids fragment derived from RUCILP1, on 3T3-L1 fibroblasts (FIG. 45), however, its effects are considered as non-significant despite clear trends in up-regulation of browning markers consisting of Ucp1, Prdm16, and Dio2.

Comparative Studies of RUCILP1 and RUCILP2 when Measured In Vivo in C57/b6 Mice

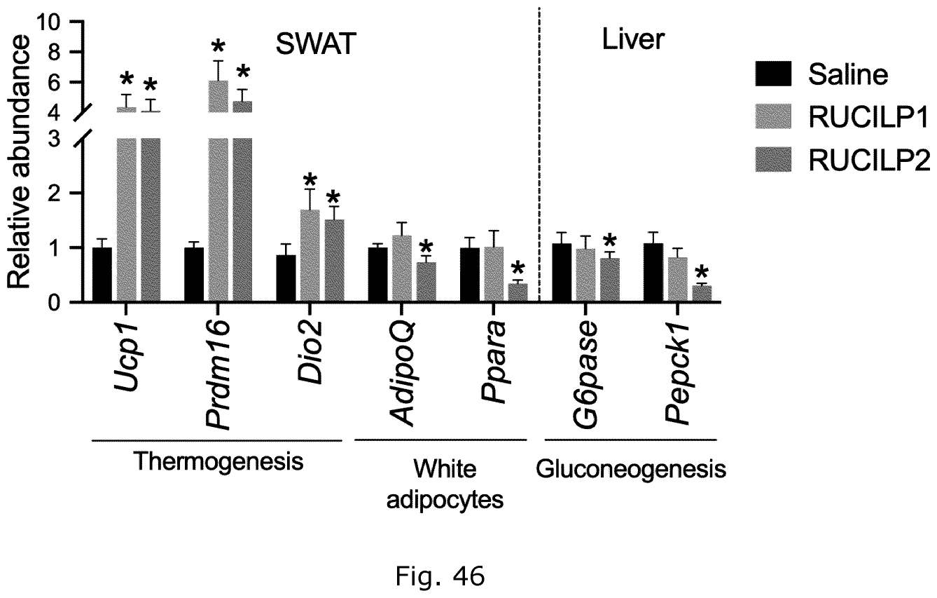

RUCILP1 or RUCILP2 was injected into peritoneum of C57/b6 mice for 7 days at a dose of 1 mg/kg of body weight and isotonic saline was given in the same manner as control. After dissection of subcutaneous white adipose tissue (SWAT), followed by quantitative PCR-based measurement of markers for thermogenesis and white adipocytes, we found both RUCILPs show comparable effects on thermogenic markers, including Ucp1, Prdm16, and Dio2 (FIG. 46). However, we did not find significant reduction of expression of gene markers for white adipocytes, i.e. AdipoQ and Ppara, upon RUCILP1 exposure, while RUCILP2 exposure diminishes expression of these two genes in white adipose cells. In mouse liver cells, RUCILP2 exposure lowered expression of genes involved in gluconeogenesis, i.e. G6pase and Pepck1, while RUCILP1 exposure had no effect.

### Conclusion

In in vitro experiments, we found that exposure of mouse fibroblasts (3T3-L1), mouse osteoblasts (MLO-Y4), and mouse myoblasts (C2C12) to recombinant RUCILP1 or RUCILP2 in equivalent concentrations induces overall similar effects on expression of selected genes.

21-AABP1 has no significant in vitro effect on expression of browning genes. In in vivo mice studies on effects of RUCILP1 and RUCILP2, both peptides have comparable stimulatory effects on thermogenesis in subcutaneous white adipose tissue (SWAT). However, while RUCILP2 reduces the expression of white genes in SWAT and gluconeogenic gene makers in liver, RUCILP1 has no such effects.

### Example 9—Alanine Scanning to Identify Specific Residues Responsible for Binding to Integrin Receptor and Truncation Scanning to Determine Minimal Length of Peptides Required for Binding Activity of RUCILP1- and RUCILP2-Derived Fragments

Following the findings that RUCILP1 and RUCILP2 may bind to integrin αV/β5 receptor via 19-mer binding epitopes, the following was undertaken: 1) Alanine scanning of a peptide library to identify the specific amino acid residues responsible for the binding; and 2) Truncation scanning of a peptide library to estimate the shortest peptide that maintains the binding activity, followed by performing SPOT peptide microarray (μSPOT) assay to visualize and quantify the binding affinity.

### Materials & Methods

### Synthesis of Alanine Scanning Library and Truncation Scanning Library of Both 19-Mer Epitopes for μSPOT Assay

μSPOT peptide arrays²² (CelluSpots, Intavis AG, Cologne, Germany) were synthesized using a RePepSL synthesizer (Intavis AG) on acid labile, amino functionalized, cellulose membrane discs (Intavis AG) containing 9-fluorenylmethy-loxycarbonyl-β-alanine (Fmoc-β-Ala) linkers (minimum loading 1.0 μmol/cm). Synthesis was initiated by Fmoc deprotection using 20% piperidine in N-methylpyrrolidone (NMP) (1×2 and 1 ×4 μL, 3 and 5 min, respectively) followed by washing with dimethylforma-mide (DMF, 7×100 μL per disc) and ethanol (EtOH, 3×300 μL per disc). The loading of the discs were reduced to 50%, by using a mixture of Fmoc-Gly-OH and Boc-Gly-OH (0.25 M: 0.25 M in NMP). All couplings were achieved using 1.2 μL of coupling solution consisting of preactivated amino acids (AAs, 0.5 M) with ethyl 2-cyano-2-(hydroxyimino)acetate oxyma (1.5 M) and N,N′-diisopropylcarbodiimide (DIC, 1.1 M) in NMP (2:1:1, AA:oxyma:DIC). The couplings were carried out 6 times (5, 10, 20, 30, 30, and 30 min, respectively), and subsequently, the membrane was capped twice with capping mixture (5% acidic anhydride in NMP), followed by washes with DMF (7×100 μL per disc). After chain elongation, final Fmoc deprotection was performed with 20% piperidine in NMP (3×4 μL, 5 min each), followed by 6× washes with DMF, subsequent N-terminal acetylation with capping mixture (3×4 μL, 5 min each) and final washes with DMF (7×100 μL per disc) and EtOH (7×200 μL per disc). Dried cellulose membrane discs were transferred to 96 deep-well blocks and were treated with the side-chain deprotection solution consisting of 80% TFA, 12% DCM, 5% H2O, and 3% TIPS (150 μL per well) for 1.5 h at room temperature (rt). Afterward, the deprotection solution was removed, and the discs were solubilized overnight at rt using a solvation mixture containing 88.5% TFA, 4% trifluoromethansufonic acid (TFMSA), 5% H2O, and 2.5% TIPS (250 μL per well). The resulting peptide-cellulose conjugates were precipitated with ice-cold ether (700 μL per well) and spun down at 1000 rpm for 90 min, followed by an additional wash of the formed pellet with ice-cold ether. The resulting pellets were redissolved in DMSO (250 μL per well) to give final stocks, which were transferred to a 384-well plate and printed (in duplicates) on white coated CelluSpots blank slides (76×26 mm, Intavis AG) using a SlideSpotter robot (Intavis AG).

### Visualization and Analysis of μSPOT Assay

After the peptide array slides were rinsed with phosphate buffered saline (PBS) (pH 7.4), the arrays were blocked with 3% bovine serum albumin (BSA) in PBS for >2 h at rt. Subsequently, the arrays were incubated with His-tagged Integrin receptor (2.5 nM) in blocking for 1 h at rt. After 5×1 min washes with blocking buffer the slides were probed with HRP-conjugated 6x-His tag antibody (1:10000, ab184607, abcam) for 0.5 h at rt. Finally, the slides were washed 3×1 min with PBS, 2×1 min with PBST, 2×1 min PBS at rt. The washed arrays visualized using SuperSignal West Femto Maximum Sensitivity Substrate (Thermo Scientific) with Fusion FX SPectra multimodal imaging platform (Vilber). The resulting blots were analyzed using the Array Analyze Software (Active Motif), which defines the error range of each data set by comparing the intensities of each peptide duplicate on the analyzed array.

### Results

To identify the amino acid residues that are critically important for the bindings between the two RUCILPs and the integrin receptor, we generated an alanine scanning library of the identified binding epitopes in both RUCILPs, where we substituted each amino acid residues of the 19-mer epitope with alanine and compared binding affinity of the peptides to the integrin receptor with that of the wild type.

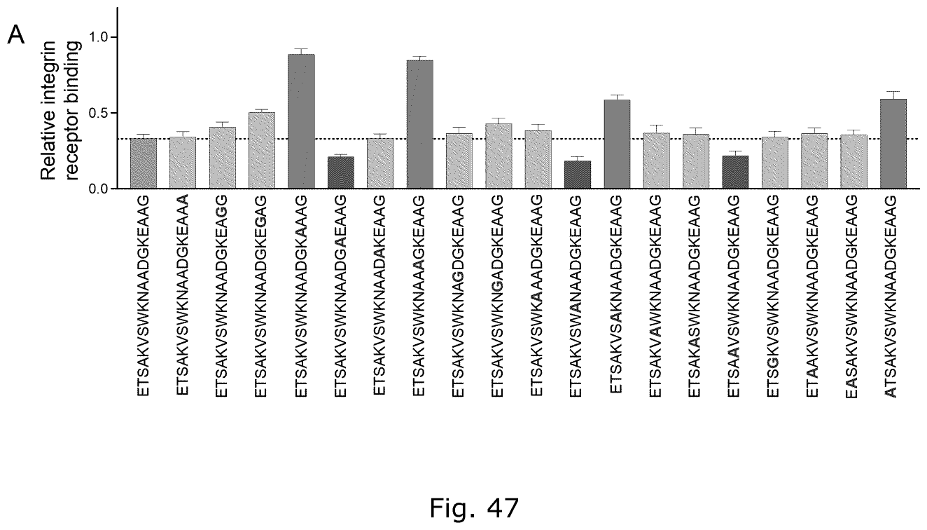

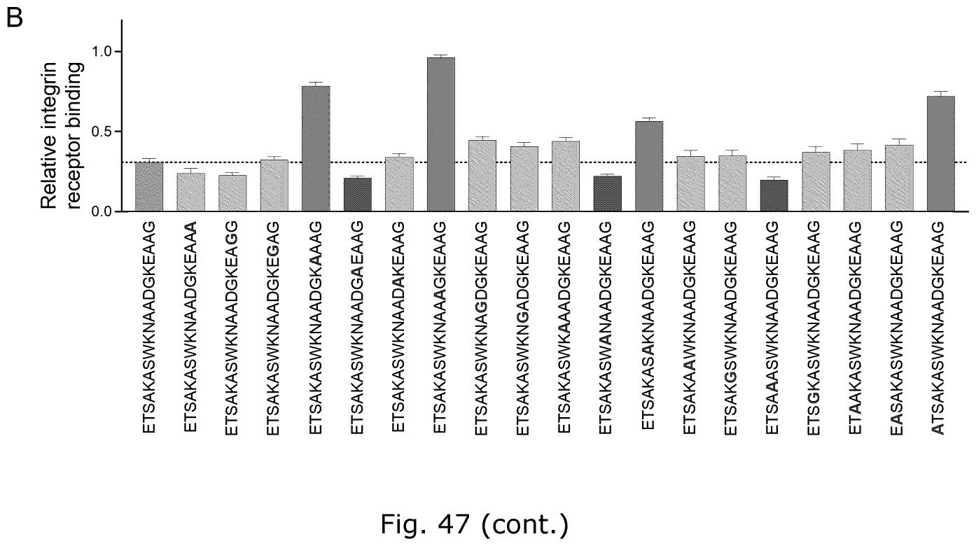

As shown in FIG. 47, after a systematic screening of the mutant peptides, we found that substitutions of lysine in both RUCILP1 and RUCILP2 resulted in a severe loss of receptor affinity, indicating the importance of basic residues within the binding epitope of both RUCILPs.

In addition, we found that substitutions of acidic residues (glutamic acid and aspartic acid), or tryptophan with alanine, resulted in increased binding affinities.

The results are interpreted as below:
(1) Amino acid residues in 19-mer epitopes that may be important for maintaining the binding affinity:

| | RUCILP1: |
|---|---|
| (SEQ ID NO: 169) | |
| | ¹²ETSAKVSWKNAADGKEAAG³⁰ |
| | RUCILP2: |
| (SEQ ID NO: 183) | |
| | ¹²ETSAKASWKNAADGKEAAG³⁰ |

(2) Amino acid residues in 19-mer epitopes that may potentially enhance binding affinity after substitution to alanine:

| | RUCILP1: |
|---|---|
| (SEQ ID NO: 169) | |
| | ¹²ETSAKVSWKNAADGKEAAG³⁰ |
| | RUCILP2: |
| (SEQ ID NO: 183) | |
| | ¹²ETSAKASWKNAADGKEAAG³⁰ |

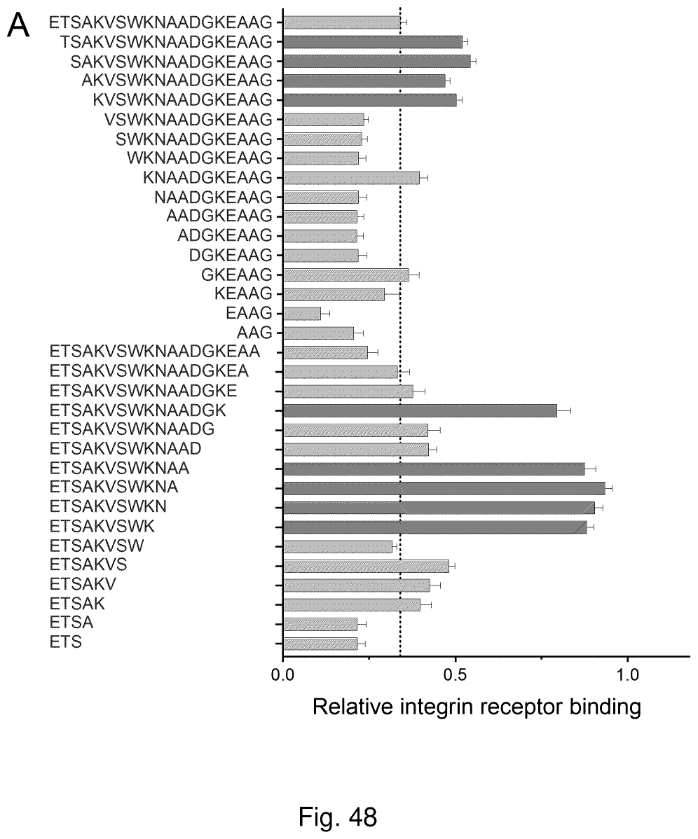

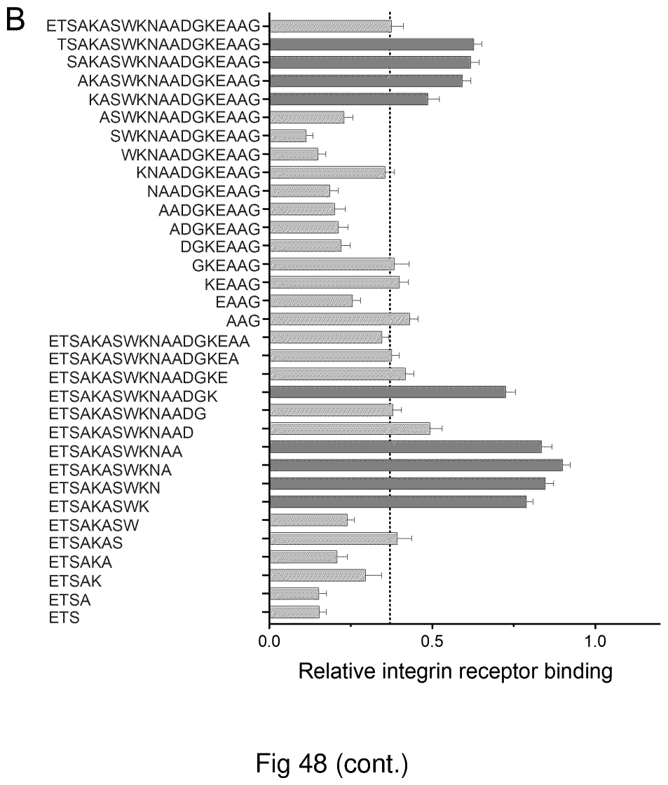

Next, we performed truncation scanning of identified binding epitopes to determine the minimal length required for maintaining core binding activity. The library was generated through a systematic truncation of the peptide's sequence from each terminus. As shown in FIG. 48, for both binding epitopes, we found that N-terminal amino acid residues are more important for maintaining core binding activity to the integrin receptor than C-terminal residues.

When compared to 19-mer peptides, 15-mer peptides show higher binding affinity, indicating that truncated peptides may display tighter binding to the integrin receptor. By employing truncation scanning, we demonstrated that the shortest peptides displaying high binding affinity are:

| | RUCILP1: |
|---|---|
| (SEQ ID NO: 235) | |
| | ¹²ETSAKVSWK²⁰ |
| | RUCILP2: |
| (SEQ ID NO: 268) | |
| | ¹²ETSAKASWK²⁰ |

### Discussion

We hereby report that the 3 lysine (K¹⁶, K²⁰, and K²⁶) residues of both 19-mer binding epitopes identified in RUCILP1 and RUCILP2 are important for maintaining the binding activity. Among the three lysine residues, the C-terminal lysine residues (K²⁶) of both 19-mer epitopes were previously predicted to be involved in the flexible loop (¹⁷ADGK²⁰) responsible for the binding to integrin receptor.

Our alanine scanning results not only support the AlphaFold-predicted findings, but also highlight the importance of basic amino acid residues, in particular lysine, for maintaining binding affinity to the integrin receptor.

Of considerable importance, we found substitution of four acidic residues resulted in a remarkable increase in binding affinity, providing candidate sites for peptide modification to improve affinity further.

In the truncation scanning, we truncated the 19-mer epitopes to peptides containing nine amino acid residues with comparably favorable binding properties. The 9-mer peptides may potentially serve as potential drug-lead analogues to both RUCILPs while retaining many of the key features of RUCILP1 and RUCILP2.

### Sequence Overview

| SEQ | |
|---|---|
| ID NO. | Description |
| 1 | Human fibronectin type III domain-containing protein 5 |
| | (FNDC5); amino acid sequence |
| 2 | Human irisin; amino acid sequence |
| 3 | Ruminococcus torques ATCC_27756, FNDC5-like precursor; |
| | amino acid sequence |
| 4 | Ruminococcus torques RUminococCus torques Irisin-Like |
| | Peptide 2 (RUCILP2); amino acid sequence |
| 5 | Polypeptide 21-AABP2 derived from RUCILP2 from |
| | Ruminococcus torques; amino acid sequence |
| 6 | Trypsin digested polypeptide derived from amino acid 7-16 |
| | of RUCILP2 from Ruminococcus torques; amino acid sequence |
| 7 | Trypsin digested polypeptide derived from amino acid 27-39 |
| | of RUCILP2 from Ruminococcus torques; amino acid sequence |
| 8 | Trypsin digested polypeptide derived from amino acid 43-56 |
| | of RUCILP2 from Ruminococcus torques; amino acid sequence |
| 9 | Ruminococcus torques RUCILP2; DNA sequence |
| 10 | Ruminococcus torques RUCILP2; DNA sequence; codon- |
| | optimized for Escherichia coli |
| 11 | Polypeptide 21-AABP derived from RUCILP2 from |
| | Ruminococcus torques; DNA sequence |
| 12 | Polypeptide 21-AABP derived from RUCILP2 from |
| | Ruminococcus torques; DNA sequence; codon-optimized for |
| | Escherichia coli |
| 13 | Trypsin digested polypeptide derived from amino acid 7-16 |
| | of RUCILP2 from Ruminococcus torques; DNA sequence |
| 14 | Trypsin digested polypeptide derived from amino acid 7-16 |
| | of RUCILP2 from Ruminococcus torques; DNA sequence; |
| | codon-optimized for Escherichia coli |
| 15 | Trypsin digested polypeptide derived from amino acid 27-39 |
| | of RUCILP2 from Ruminococcus torques; DNA sequence |
| 16 | Trypsin digested polypeptide derived from amino acid 27-39 |
| | of RUCILP2 from Ruminococcus torques; DNA sequence; |
| | codon-optimized for Escherichia coli |
| 17 | Trypsin digested polypeptide derived from amino acid 43-56 |
| | of RUCILP2 from Ruminococcus torques; DNA sequence |
| 18 | Trypsin digested polypeptide derived from amino acid 43-56 |
| | of RUCILP2 from Ruminococcus torques; DNA sequence; |
| | codon-optimized for Escherichia coli |
| 19 | Ruminococcus torques RUminococCus torques Irisin-Like |
| | Peptide 1 (RUCILP1); amino acid sequence |
| 20 | Polypeptide 21-AABP1 derived from RUCILP1 from |
| | Ruminococcus torques; amino acid sequence |
| 21 | Ruminococcus torques ATCC_27756, RUMTOR_00181 |
| | (Uniprot: A5KIY5); amino acid sequence |
| 22-95 | 15-mer overlapping fragments of RUCILP1; amino acid |
| | sequence |
| 96-168 | 15-mer overlapping fragments of RUCILP2; amino acid |
| | sequence |
| 169-188 | Alanine scanning sequences of RUCILP1; amino acid sequence |
| 189-208 | Alanine scanning sequences of RUCILP2; amino acid sequence |
| 209-241 | Truncation scanning sequences of RUCILP1; amino acid |
| | sequence |
| 242-274 | Truncation scanning sequences of RUCILP2; amino acid |
| | sequence |

### REFERENCES

1. Lynch, S. V. & Pedersen, O. The human intestinal microbiome in health and disease. N. Engl. J. Med. 375, 2369-2379 (2016).
2. Fan, Y. & Pedersen, O. Gut microbiota in human metabolic health and disease. Nat. Rev. Microbiol. 19, 55-71 (2021).
3. Qin, J. et al. A human gut microbial gene catalogue established by metagenomic sequencing. Nature 464, 59-65 (2010).
4. Plovier, H. et al. A purified membrane protein from Akkermansia muciniphila or the pasteurized bacterium improves metabolism in obese and diabetic mice. Nat. Med. 23, 107-113 (2017).
5. Depommier, C. et al. Supplementation with Akkermansia muciniphila in overweight and obese human volunteers: a proof-of-concept exploratory study. Nat. Med. 25, 1096-1103 (2019).
6. NCBI Fibronectin type III domain-containing protein 5 isoform 2 preproprotein [Homo sapiens]. NCBI (2016).
7. Bostrom, P. et al. A PGC1-α-dependent myokine that drives brown-fat-like development of white fat and thermogenesis. Nature 481, 463-468 (2012).
8. Kraal, L., Abubucker, S., Kota, K., Fischbach, M. A. & Mitreva, M. The prevalence of species and strains in the human microbiome: a resource for experimental efforts. PLoS ONE 9, e97279 (2014).
9. Kim, H. et al. Irisin mediates effects on bone and fat via aV integrin receptors. Cell 175, 1756-1768. e1717 (2018).
10. Nie, Y. & Liu, D. N-Glycosylation is required for FDNC5 stabilization and irisin secretion. Biochem. J. 474, 3167-3177 (2017).
11. Schumacher, M. A., Chinnam, N., Ohashi, T., Shah, R. S. & Erickson, H. P. The structure of irisin reveals a novel intersubunit β-sheet fibronectin type III (FNIII) dimer: implications for receptor activation. J. Biol. Chem. 288, 33738-33744 (2013).
12. Khanal, P., Jia, Z. & Yang, X. Cysteine residues are essential for dimerization of Hippo pathway components YAP2L and TAZ. Sci. Rep. 8, 1-12 (2018).
13. Ronda, L., Bruno, S., Bettati, S., Storici, P. & Mozzarelli, A. From protein structure to function via single crystal optical spectroscopy. Front. Mol. Biosci. 2, 12 (2015).
14. Schumacher, M. A., Chinnam, N., Ohashi, T., Shah, R. S. & Erickson, H. P. The Structure of Irisin Reveals a Novel Intersubunit β-Sheet Fibronectin Type III (FNIII) Dimer. J. Biol. Chem. 288, 33738-33744 (2013).
15. Albrecht, E. et al. Irisin: Still chasing shadows. Mol. Metab. 34, 124-135 (2020).
16. Roth, Z., Yehezkel, G. & Khalaila, I. Identification and quantification of protein glycosylation. Int. J. Carbohydr. Chem. 2012 (2012).
17. Li, D. et al. Distinct functions of PPARγ isoforms in regulating adipocyte plasticity. Biochem. Biophys. Res. Commun. 481, 132-138 (2016).
18. Li, W. et al. High potency of a bivalent human VH domain in SARS-CoV-2 animal models. Cell 183, 429-441. e416 (2020).
19. Zhang, D. et al. Review of Research on the Role of Irisin in Tumors. OncoTargets Ther. 13, 4423 (2020).
20. Christiansen, C. B. et al. The impact of short-chain fatty acids on GLP-1 and PYY secretion from the isolated perfused rat colon. Am. J. Physiol. Gastrointest. Liver Physiol. 315, G53-G65 (2018).
21. Liu, T.-Y. et al. Irisin inhibits hepatic gluconeogenesis and increases glycogen synthesis via the P13K/Akt pathway in type 2 diabetic mice and hepatocytes. Clin. Sci. 129, 839-850 (2015).
22. Jumper, J. et al. Highly accurate protein structure prediction with AlphaFold. Nature 596, 583-589 (2021).
23. Mirdita, M. et al. ColabFold-Making protein folding accessible to all. (2021).
24. Steinegger, M. & Söding, J. MMseqs2 enables sensitive protein sequence searching for the analysis of massive data sets. Nat. Biotechnol. 35, 1026-1028 (2017).
25. Dikmans, A., Beutling, U., Schmeisser, E., Thiele, S. & Frank, R. SC2: A novel process for manufacturing multipurpose high-density chemical microarrays. Qsar Comb Sci 25, 1069-1080, doi:10.1002/qsar.200640130 (2006).
26. Schumacher, M. A., Chinnam, N., Ohashi, T., Shah, R. S. & Erickson, H. P. The structure of Irisin reveals a novel intersubunit β-sheet fibronectin type III (FNIII) dimer: Implications for receptor activation. J. Biol. Chem. 288, 33738-33744, doi:10.1074/jbc.M113.516641 (2013).

1-36. (canceled)

37. An isolated polypeptide having a length of less than 200 amino acids comprising or consisting of an amino acid sequence selected from the group consisting of:
a. the amino acid sequence according to SEQ ID NO: 4 or SEQ ID NO: 19;
b. a variant of SEQ ID NO: 4 or SEQ ID NO: 19, wherein said variant has at least 60%, but less than 99% sequence identity to SEQ ID NO: 4 or SEQ ID NO: 19;
c. a variant of SEQ ID NO: 4 or SEQ ID NO: 19, wherein said variant has between 1 and 40 amino acid substitutions relative to SEQ ID NO: 4 or SEQ ID NO: 19;
d. a fragment of SEQ ID NO: 4 or SEQ ID NO: 19 having a length of at least 10 amino acids, or a variant of said fragment having between 1 and 5 amino acid substitutions relative to SEQ ID NO: 4 or SEQ ID NO: 19, respectively, wherein said polypeptide has a length of less than 50 amino acids;
e. an amino acid sequence differing from SEQ ID NO: 4 or SEQ ID NO: 19 by truncation at the N-terminus by between 1-67 amino acids, or a variant thereof having between 1 and 10 amino acid substitutions relative to SEQ ID NO: 4 or SEQ ID NO: 19;
f. an amino acid sequence differing from SEQ ID NO: 4 or SEQ ID NO: 19 by truncation at the C-terminus by between 1-21 amino acids, or a variant thereof having between 1 and 30 amino acid substitutions relative to SEQ ID NO: 4 or SEQ ID NO: 19;
g. an amino acid sequence differing from SEQ ID NO: 4 or SEQ ID NO: 19 by truncation at the N-terminus by between 1-67 amino acids, wherein said polypeptide has a length of at least 10 amino acids, or a variant thereof having between 1 and 5 amino acid substitutions relative to SEQ ID NO: 4/or SEQ ID NO: 19;
h. the amino acid sequence according to SEQ ID NO: 5 or SEQ ID NO: 20;
i. a variant of SEQ ID NO: 5 or SEQ ID NO: 20, wherein said variant has at least 70%, but less than 99% sequence identity to SEQ ID NO: 5 or SEQ ID NO: 20;
j. a variant of SEQ ID NO: 5 or SEQ ID NO: 20, wherein said variant has between 1 and 10 amino acid substitutions relative to SEQ ID NO: 5 or SEQ ID NO: 20, wherein said polypeptide has a length of less than 50 amino acids;
k. a fragment of SEQ ID NO: 5 or SEQ ID NO: 20 comprising at least 10 consecutive amino acids of SEQ ID NO: 5 or SEQ ID NO: 20, or a variant thereof having between 1 and 5 amino acid substitutions relative to SEQ ID NO: 5 or SEQ ID NO: 20, wherein said polypeptide has a length of less than 50 amino acids;
l. a fragment of SEQ ID NO: 19, wherein said fragment is selected from the group consisting of SEQ ID NOs: 27, 33, 34, 35, 36, 37 and 95, and respective variants thereof having between 1 and 3 amino acid substitutions relative to SEQ ID NO: 19, wherein said polypeptide has a length of less than 50 amino acids;
m. a fragment of SEQ ID NO: 4, wherein said fragment is selected from the group consisting of SEQ ID NOs: 107, 108, 109, 110, 111, 165 and 168, and respective variants thereof having between 1 and 3 amino acid substitutions relative to SEQ ID NO: 4, wherein said polypeptide has a length of less than 50 amino acids;
n. a fragment of a variant of SEQ ID NO: 19, wherein said fragment is selected from the group consisting of SEQ ID NOs: 173, 176, 181 and 188, and respective variants thereof having between 1 and 3 amino acid substitutions relative to SEQ ID NO: 19, wherein said polypeptide has a length of less than 50 amino acids;
o. a fragment of a variant of SEQ ID NO: 4, wherein said fragment is selected from the group consisting of SEQ ID NOs: 193, 196, 201 and 208, and respective variants thereof having between 1 and 3 amino acid substitutions relative to SEQ ID NO: 4, wherein said polypeptide has a length of less than 50 amino acids;
p. a fragment of SEQ ID NO: 19, wherein said fragment is selected from the group consisting of SEQ ID NOs: 210, 211, 212, 213, 229, 232, 233, 234 and 235, and respective variants thereof having between 1 and 3 amino acid substitutions relative to SEQ ID NO: 19, wherein said polypeptide has a length of less than 50 amino acids; and
q. a fragment of SEQ ID NO: 4, wherein said fragment is selected from the group consisting of SEQ ID NOs: 243, 244, 245, 246, 262, 265, 266, 267 and 268, and respective variants thereof having between 1 and 3 amino acid substitutions relative to SEQ ID NO: 4, wherein said polypeptide has a length of less than 50 amino acids.

38. The polypeptide according to claim 37, wherein the polypeptide has a length of at least 10 amino acids.

39. The polypeptide according to claim 37, wherein the polypeptide has a length of less than 100 amino acids.

40. The polypeptide according to claim 37, wherein the polypeptide has a length of 10-200 amino acids.

41. The polypeptide according to claim 37, wherein the variant has at least 90% sequence identity to SEQ ID NO: 4 or SEQ ID NO: 19.

42. The polypeptide according to claim 37, wherein the variant has at least 90% sequence identity to SEQ ID NO: 5 or SEQ ID NO: 20.

43. The polypeptide according to claim 37, wherein the amino acid substitutions are conservative substitutions.

44. The polypeptide according to claim 37, wherein said fragment comprises or consists of:
a. an amino acid sequence according to positions 7 to 16 of SEQ ID NO: 4, corresponding to SEQ ID NO: 6, or a variant thereof having between 1 and 5 amino acid substitutions as compared to SEQ ID NO: 4; or
b. an amino acid sequence according to positions 27 to 39 of SEQ ID NO: 4, corresponding to SEQ ID NO: 7, or a variant thereof having between 1 and 6 amino acid substitutions as compared to SEQ ID NO: 4; or
c. an amino acid sequence according to positions 43 to 56 of SEQ ID NO: 4, corresponding to SEQ ID NO: 8, or a variant thereof having between 1 and 6 amino acid substitutions as compared to SEQ ID NO: 4.

45. The polypeptide according to claim 37, wherein the polypeptide comprises:
a. a V at amino acid position 7 of SEQ ID NO: 4, or a conservative substitution thereof, or a E at amino acid position 9 of SEQ ID NO: 4, or a conservative substitution thereof, or a E at amino acid position 58 of SEQ ID NO: 4, or a conservative substitution thereof, or
b. a Y at amino acid position 5 of SEQ ID NO: 5, or a conservative substitution thereof, or a F at amino acid position 6 of SEQ ID NO: 5, or a conservative substitution thereof, or a E at amino acid position 8 of SEQ ID NO: 5, or a conservative substitution thereof; or a N at amino acid position 17 or a conservative substitution thereof.

46. A conjugate comprising the polypeptide according to claim 37, wherein said polypeptide comprises one or more moieties conjugated to said polypeptide.

47. The conjugate according to claim 46, wherein the one or more moieties are selected from alkyls, aryls, heteroaryls, olefins, fatty acids, polyethylene glycol (PEG), saccharides, and polysaccharides.

48. The polypeptide according to claim 37, wherein said polypeptide is dimer or a multimer.

49. The polypeptide according to claim 37, wherein the polypeptide is capable of:
a. binding to the αV/β05 integrin receptor;
b. inducing thermogenesis in white adipocytes;
c. reducing the lipid content of adipocytes;
d. stimulating bone formation;
e. inducing cardiomyogenesis;
f. inducing myotube formation and myogenesis in myoblasts;
g. enhancing the intestinal barrier junction;
h. stimulating secretion of glucagon like peptide-1 (GLP-1) and glucagon like peptide-2 (GLP-2);
i. stimulating secretion of insulin;
j. stimulating secretion of peptide-YY (PYY);
k. stimulating secretion of somatostatin;
l. inducing weight loss;
m. improving glucose tolerance; or
n. increasing the cortical thickness of the tibia bone.

50. An isolated polynucleotide encoding the polypeptide according to claim 37.

51. A vector comprising the polynucleotide according to claim 50.

52. A host cell comprising the polynucleotide according to claim 50.

53. A pharmaceutical composition comprising:
a. the isolated polypeptide of claim 37; or
b. a RUMTOR_00181 polypeptide comprising or consisting of
i. the polypeptide according to SEQ ID NO: 21; or
ii. a variant of SEQ ID NO: 21 with at least 85% sequence identity thereto; or
c. an isolated polynucleotide encoding the isolated polypeptide of claim 37; or
d. a polynucleotide encoding said RUMTOR_00181 polypeptide; or
e. a vector comprising a polynucleotide encoding the isolated polypeptide of claim 37; or
f. a vector comprising the polynucleotide encoding said RUMTOR_00181 polypeptide; or
g. a host cell comprising a polynucleotide encoding the isolated polypeptide of claim 37; or
h. a host cell comprising:
i. a polynucleotide encoding said RUMTOR_00181 polypeptide; or
ii. a vector comprising the polynucleotide encoding said RUMTOR_00181 polypeptide.

54. A method of inducing weight loss, improving glucose tolerance, reducing body fat mass, increasing lean body mass, reducing the lipid content of adipocytes, inducing thermogenesis in white adipocytes, increasing the cortical thickness of the tibia bone or stimulating bone formation, wherein the method comprises administering the pharmaceutical composition of claim 53 to an individual in need thereof.

55. A method for the treatment of metabolic disorders, muscle disorders and injuries, or bone disorders, wherein the method comprises administering to a subject in need thereof the pharmaceutical composition of claim 53.

56. The method according to claim 55, wherein the metabolic disorder, muscle disorder and injury, or bone disorder is selected from metabolic syndrome, obesity, prediabetes, T2D, FLD, cardiovascular disease, muscular dystrophy, Duchenne muscular dystrophy, ALS, Lambert-Eaton syndrome, myasthenia gravis, polymyositis, peripheral neuropathy, osteoporosis, osteogenesis imperfect and osteopetrosis.
